(19) United States
(12) Patent Application Publication
(10) Pub. No.: US 2017/0137890 A1
(43) Pub. Date: May 18, 2017

(54) CANCER PROGNOSIS SIGNATURES

(71) Applicant: Myriad Genetics, Inc., Salt Lake City, UT (US)

(72) Inventors: Jerry Lanchbury, Salt Lake City, UT (US); Alexander Gutin, Salt Lake City, UT (US); Darl Flake, Salt Lake City, UT (US)

(21) Appl. No.: 15/331,076

(22) Filed: Oct. 21, 2016

Related U.S. Application Data
(63) Continuation of application No. PCT/US2015/027091, filed on Apr. 22, 2015.
(60) Provisional application No. 61/983,366, filed on Apr. 23, 2014.

(51) Int. Cl. C12Q 1/68; G06F 19/24

(52) U.S. Cl. CPC C12Q 1/6886; G06F 19/24; C12Q 2600/158; C12Q 2600/118; C12Q 2600/106

(57) ABSTRACT

The disclosure provides for molecular classification of disease and, particularly, molecular markers for breast cancer prognosis and methods and systems of use thereof.

### CROSS-REFERENCE TO RELATED APPLICATIONS

This application claims priority to Patent Cooperation Treaty International Application Serial No. PCT/US2015/027091 filed Apr. 22, 2015, which claims priority to U.S. provisional application Ser. No. 61/983,366, filed Apr. 23, 2014, the contents of which are hereby incorporated by reference in their entirety.

### FIELD OF THE INVENTION

This disclosure generally relates to a molecular classification of cancer and particularly to molecular markers for cancer prognosis and methods of use thereof.

### BACKGROUND OF THE INVENTION

Cancer is a major public health problem, accounting for roughly 25% of all deaths in the United States. American Cancer Society, FACTS AND FIGURES. 2010. Though many treatments have been devised for various cancers, these treatments often vary in severity of side effects. It is useful for clinicians to know how aggressive a patient's cancer is in order to determine how aggressively to treat the cancer.

### SUMMARY OF THE INVENTION

The inventors have discovered gene expression signatures related to classifying cancer. Classifying cancer using these signatures can include prediction of prognosis for survival (e.g., distant metastasis-free survival), treating cancer, monitoring cancer, selection of therapeutic treatments or regimens, and such. In particular, a set of genes related to the immune system (herein referred to as “immune system genes” or “ISGs” or “ISG” in the singular) and a set of other genes related to cancer prognosis (herein referred to as “Other Cancer Prognostic Genes” or “OCPGs” or “OCPG” in the singular) were identified as a result of these studies. Remarkably, these genes have predictive power for classifying cancer.

The genes identified in these studies include immune systems genes, or ISGs, that for convenience can further be subdivided into three sub-groups based on their general biological characteristics: B-cell related genes (“BCRGs” or “BCRG” in the singular), T-cell related genes (“TCRGs” or “TCRG” in the singular) and HLA class II activation-related genes (“HLAGs” or “HLAG” in the singular). The ISGs are genes whose higher or increased expression is associated with a good or better prognosis and lower or no increase in expression is associated with a worse prognosis. The BCRGs, which are genes that are typically expressed in B-cells, were found to be expressed in cancer cells from patients and found to have prognostic value in these studies. The TCRGs, which are genes that are typically expressed in T-cells, were found to be expressed in cancer cells from patients and found to have prognostic value in these studies. The HLAGs, which are genes that are typically related to HLA class II activation, were found to be expressed in cancer cells from patients and found to have prognostic value in these studies. These genes are very useful for classifying cancer. As described in more detail below sets of genes selected from the BCRGs, TCRGs, and HLAGs, alone, or when added to other gene expression profiles such as cell cycle gene expression profiles, or the OCPGs, yield highly predictive signatures for cancer classification.

Another group of genes found to be useful for cancer classification, the OCPGs, were identified in these studies. These genes are very useful for, e.g., predicting survival (e.g., distant metastasis free survival) in cancer patients. OCPGs can be further subdivided into two subgroups: one subgroup has genes whose higher expression is associated with a better prognosis (bpOCPGs or “better prognosis Other Cancer Prognostic Genes”) and another subgroup that has genes whose higher expression is associated with worse prognosis (wpOCPGs or “worse prognosis Other Cancer Prognostic Genes”). Unlike ISGs, the OCPGs are genes with no clear linking biochemical tie as a group, which were found to be expressed in cancer cells from patients and found to have prognostic value in these studies. As described in more detail below sets of genes selected from the OCPGs, alone, or when added to other gene expression profiles such as the cell cycle gene expression profiles or the genes from the BCRGs, TCRGs, or HLAGs yield highly predictive signatures for cancer classification.

The inventors previously discovered that the expression of those genes whose expression closely tracks the cell cycle (“cell-cycle genes,” “CCGs,” or “CCP genes” as further defined below) is particularly useful in classifying various cancers including e.g., breast cancer and prostate cancer. See WO/2010/080933 (also corresponding U.S. application Ser. No. 13/177,887) and WO/2012/006447 (also related U.S. application Ser. No. 13/178,380), each of which is incorporated herein by reference. The inventors have discovered a group of genes (and related probes for determining their status) in the present disclosure that is similarly prognostic in cancer (e.g., Panels A-N in Tables 1-23; Panel O in Table 34; Immune Panels 1-3; Combined Panel 1 in Table 39; Combined Panel 2 in Table 40). It has now been remarkably discovered that the expression of certain additional genes, e.g., genes from the BCRGs, TCRGs, HLAGs, and OCPGs, are prognostic on their own, and add significant prediction power to CCG expression signatures in the prognosis of cancer. For example, the p-value for predicting distant metastasis free survival for ER+ breast cancer patients when taking into account the genes descried herein and a set of CCGs was 3.5×10⁻²¹ in one of the Examples described below. In addition, it has been discovered that the expression of CCGs and certain additional genes can be used on their own to predict (or diagnose likelihood of) chemotherapy response, and add significant prediction power to CCG expression signatures in the prediction of chemotherapy response.

Accordingly, in one aspect, the present disclosure provides a method for determining gene expression in a sample from a patient identified as having cancer. Generally, the method includes at least the following steps: (1) obtaining, or providing, one or more samples from a patient identified as having cancer; (2) determining the expression of a panel of genes in said sample(s) including at least 2, 3, 4, 5, 6, 7, 8, 9, 10, 12, 14, 15, 16, 18, 20, 25, 30, 35, 40 or more genes selected from BCRGs, TCRGs, HLAGs, or OCPGs (e.g., selected from the genes listed in Tables 1-6b or Immune Panel 1, 2 and/or 3 or Immune Panel 1, 2 and/or 3); and (3) providing a test value by (a) weighting the determined expression of each of a plurality of test genes selected from said panel of genes with a predefined coefficient, and (b) combining the weighted expression to provide said test value, wherein at least 5%, at least 10%, at least 25%, at least 50%, at least 75% or at least 90% of said plurality of test genes are chosen from BCRGs, TCRGs, HLAGs, or OCPGs (or wherein BCRGs, TCRGs, HLAGs, or OCPGs represent at least 5%, at least 10%, at least 25%, at least 50%, at least 75% or at least 85% of the combined weight used to provide the test value). In a specific aspect, the cancer is lung cancer, bladder cancer, prostate cancer, brain cancer, or breast cancer. In another specific aspect, the cancer is breast cancer. In yet another specific aspect, the cancer is ER positive breast cancer.

Accordingly, in a related aspect, the present disclosure provides a method for determining gene expression in a sample from a patient identified as having cancer. Generally, the method includes at least the following steps: (1) obtaining, or providing, one or more samples from a patient identified as having cancer; (2) determining the expression of a panel of genes in said sample(s) including (a) at least 1, 2, 3, 4, 5, 6, 7, 8, 9, 10, 12, 14, 15, 16, 18, 20, 25, 30, 35, 40 or more cell-cycle genes and (b) at least 1, 2, 3, 4, 5, 6, 7, 8, 9, 10, 12, 14, 15, 16, 18, 20, 25, 30, 35, 40 or more genes selected from BCRGs, TCRGs, HLAGs, or OCPGs; and (3) providing a test value by (a) weighting the determined expression of each of a plurality of test genes selected from said panel of genes with a predefined coefficient, and (b) combining the weighted expression to provide said test value, wherein at least 50%, at least 75% or at least 90% of said plurality of test genes are cell-cycle genes, BCRGs, TCRGs, HLAGs, or OCPGs (or wherein cell-cycle genes, BCRGs, TCRGs, HLAGs, or OCPGs represent at least 50%, at least 75% or at least 85% of the combined weight used to provide the test value). In a specific aspect, the cancer is lung cancer, bladder cancer, prostate cancer, brain cancer, or breast cancer. In another specific aspect, the cancer is breast cancer. In yet another specific aspect, the cancer is ER positive breast cancer.

In another aspect, the present disclosure provides a method for classifying cancer in a patient (e.g., determining the patient's prognosis or the likelihood of cancer recurrence in the patient), which comprises: determining in a sample (e.g., tumor sample) from the patient the expression of at least 2, 3, 4, 5, 6, 7, 8, 9, 10, 12, 14, 15, 16, 18, 20, 25, 30, 35, 40 or more genes selected from BCRGs, TCRGs, HLAGs, or OCPGs (e.g., selected from the genes listed in Tables 1-6b or Immune Panel 1, 2 and/or 3) and using the expression of the genes in classifying the cancer (e.g., determining the prognosis of the cancer in the patient, or predicting the cancer outcome, the likelihood of response to chemotherapy, the likelihood of cancer recurrence or probability of post-surgery distant metastasis-free survival). In a specific aspect, the cancer is lung cancer, bladder cancer, prostate cancer, brain cancer, or breast cancer. In another specific aspect, the cancer is breast cancer. In yet another specific aspect, the cancer is ER positive breast cancer.

In another aspect, the present disclosure provides a method for classifying cancer in a patient (e.g., determining the patient's prognosis or the likelihood of cancer recurrence in the patient), which comprises: (a) determining in a sample (e.g., tumor sample) from the patient the expression of (1) at least 1, 2, 3, 4, 5, 6, 7, 8, 9, 10, 12, 14, 15, 16, 18, 20, 25, 30, 35, 40 or more genes selected from BCRGs, TCRGs, HLAGs, or OCPGs (e.g., selected from the genes listed in Tables 1-6b or Immune Panel 1, 2 and/or 3), and (2) and at least 1, 2, 3, 4, 5, 6, 7, 8, 9, 10, 12, 14, 15, 16, 18, 20, 25, 30, 35, 40 or more cell-cycle genes (e.g., selected from the genes listed in Table 7), and (b) using the expression of the genes selected from BCRGs, TCRGs, HLAGs, or OCPGs, and cell-cycle genes in classifying the cancer (e.g., determining the prognosis of the cancer in the patient, or predicting the cancer outcome, the likelihood of cancer recurrence, the likelihood of response to chemotherapy, or probability of post-surgery distant metastasis-free survival). In a specific aspect, the cancer is lung cancer, bladder cancer, prostate cancer, brain cancer, or breast cancer. In another specific aspect, the cancer is breast cancer. In yet another specific aspect, the cancer is ER positive breast cancer.

In another aspect, the present disclosure provides a method for classifying cancer in a patient (e.g., determining the patient's prognosis or the likelihood of cancer recurrence in the patient), which comprises: (1) determining in a sample (e.g., tumor sample) from the patient the expression of the PGR gene and at least 1, 2, 3, 4, 5, 6, 7, 8, 9, 10, 12, 14, 15, 16, 18, 20, 25, 30, 35, 40 or more genes selected from BCRGs, TCRGs, HLAGs, or OCPGs (e.g., selected from the genes listed in Tables 1-6b or Immune Panel 1, 2 and/or 3) and (2) using the expression of the PGR gene and the genes selected from BCRGs, TCRGs, HLAGs, or OCPGs in classifying the cancer (e.g., determining the prognosis of the cancer in the patient, or predicting the cancer outcome, the likelihood of response to chemotherapy, the likelihood of cancer recurrence or probability of post-surgery distant metastasis-free survival). In some embodiments, the expression of the ESR1 gene has been determined (e.g., to determine or confirm the breast cancer is ER+ or ER−). In some embodiments, the patient is ER+ and node negative. In some embodiments, the patient is ER+ and node negative, has undergone surgery to remove some or all of the tumor, and is placed on hormone therapy. In some embodiments, the method further comprises determining whether the patient has undergone hormonal therapy. In these embodiments, if the patient has undergone hormonal therapy, then the method further comprises correlating increased PGR expression to better prognosis. Conversely, if the patient has not undergone hormonal therapy, then the method further comprises correlating increased PGR expression to worse prognosis. In some embodiments, the method comprises correlating increased PGR expression to an increased likelihood of response to hormonal therapy.

In another aspect, the present disclosure provides a method for classifying cancer in a patient (e.g., determining the patient's prognosis or the likelihood of cancer recurrence in the patient), which comprises: (1) determining in a sample (e.g., tumor sample) from the patient the expression of the PGR gene, and/or the ABCC5 gene, and at least 1, 2, 3, 4, 5, 6, 7, 8, 9, 10, 12, 14, 15, 16, 18, 20, 25, 30, 35, 40 or more genes selected from BCRGs, TCRGs, HLAGs, or OCPGs (e.g., selected from the genes listed in Tables 1-6b or Immune Panel 1, 2 and/or 3) and (2) using the expression of the PGR gene, and, or the ABCC5 gene and the genes selected from BCRGs, TCRGs, HLAGs, or OCPGs in classifying the cancer (e.g., determining the prognosis of the cancer in the patient, or predicting the cancer outcome, the likelihood of response to chemotherapy, the likelihood of cancer recurrence or probability of post-surgery distant metastasis-free survival). In some embodiments, the expression of the ESR1 gene has been determined (e.g., to determine or confirm the breast cancer is ER+ or ER−). In some embodiments, the patient is ER+ and node negative. In some embodiments, the patient is ER+ and node negative, has undergone surgery to remove some or all of the tumor in her breast, and is placed on hormone therapy. In some embodiments, the method further comprises determining whether the patient has undergone hormonal therapy. In these embodiments, if the patient has undergone hormonal therapy, then the method further comprises correlating increased PGR expression to better prognosis. Conversely, if the patient has not undergone hormonal therapy, then the method further comprises correlating increased PGR expression to worse prognosis. In some embodiments, the method comprises correlating increased PGR expression to an increased likelihood of response to hormonal therapy. In some embodiments, the method comprises correlating increased ABCC5 expression to worse prognosis.

In another aspect, the present disclosure provides a method for classifying cancer in a patient (e.g., determining the patient's prognosis, the likelihood of cancer recurrence in the patient, or the likelihood of response to chemotherapy), which comprises: (1) determining in a sample (e.g., tumor sample) from the patient the expression of the PGR gene, at least 1, 2, 3, 4, 5, 6, 7, 8, 9, 10, 12, 14, 15, 16, 18, 20, 25, 30, 35, 40 or more genes selected from BCRGs, TCRGs, HLAGs, or OCPGs (e.g., selected from the genes listed in Tables 1-6b or Immune Panel 1, 2 and/or 3), and at least 1, 2, 3, 4, 5, 6, 7, 8, 9, 10, 12, 14, 15, 16, 18, 20, 25, 30, 35, 40 or more cell-cycle genes (e.g., selected from the genes listed in Table 7) and (2) using the expression of the expression of the PGR gene, the genes selected from BCRGs, TCRGs, HLAGs, or OCPGs, and the cell-cycle genes in classifying the cancer (e.g., determining the prognosis of the cancer in the patient, or predicting the cancer outcome, the likelihood of response to chemotherapy, the likelihood of cancer recurrence or probability of post-surgery distant metastasis-free survival). In some embodiments, the expression of the ESR1 gene has been determined (e.g., to determine or confirm the breast cancer is ER+ or ER−). In some embodiments, the patient is ER+ and node negative. In some embodiments, the patient is ER+ and node negative, has undergone surgery to remove some or all of the tumor, and is placed on hormone therapy. In some embodiments, the method further comprises determining whether the patient has undergone hormonal therapy. In these embodiments, if the patient has undergone hormonal therapy, then the method further comprises correlating increased PGR expression to better prognosis. Conversely, if the patient has not undergone hormonal therapy, then the method further comprises correlating increased PGR expression to worse prognosis. In some embodiments, the method comprises correlating increased PGR expression to an increased likelihood of response to hormonal therapy.

In another aspect, the present disclosure provides a method for classifying cancer in a patient (e.g., determining the patient's prognosis, the likelihood of cancer recurrence in the patient, or the likelihood of response to chemotherapy), which comprises: (1) determining in a sample (e.g., tumor sample) from the patient the expression of the PGR gene, and, or the ABCC5 gene, and at least 1, 2, 3, 4, 5, 6, 7, 8, 9, 10, 12, 14, 15, 16, 18, 20, 25, 30, 35, 40 or more genes selected from BCRGs, TCRGs, HLAGs, or OCPGs (e.g., selected from the genes listed in Tables 1-6b or Immune Panel 1, 2 and/or 3), and at least 1, 2, 3, 4, 5, 6, 7, 8, 9, 10, 12, 14, 15, 16, 18, 20, 25, 30, 35, 40 or more cell-cycle genes (e.g., selected from the genes listed in Table 7) and (2) using the expression of the expression of the PGR gene, and, or the ABCC5 gene, the genes selected from BCRGs, TCRGs, HLAGs, or OCPGs, and the cell-cycle genes in classifying the cancer (e.g., determining the prognosis of the cancer in the patient, or predicting the cancer outcome, the likelihood of response to chemotherapy, the likelihood of cancer recurrence or probability of post-surgery distant metastasis-free survival). In some embodiments, the expression of the ESR1 gene has been determined (e.g., to determine or confirm the patient is ER+ or ER−). In some embodiments, the patient is ER+ and node negative. In some embodiments, the patient is ER+ and node negative, has undergone surgery to remove some or all of the tumor in her breast, and is placed on hormone therapy. In some embodiments, the method further comprises determining whether the patient has undergone hormonal therapy. In these embodiments, if the patient has undergone hormonal therapy, then the method further comprises correlating increased PGR expression to better prognosis. Conversely, if the patient has not undergone hormonal therapy, then the method further comprises correlating increased PGR expression to worse prognosis. In some embodiments, the method comprises correlating increased PGR expression to an increased likelihood of response to hormonal therapy. In some embodiments, the method comprises correlating increased ABCC5 expression with worse prognosis.

Clinical parameters can be combined with the information gained from analysis of BCRGs, TCRGs, HLAGs, or OCPGs. Thus, in yet another aspect, the present disclosure provides a method for classifying cancer in a patient (e.g., determining the patient's prognosis, the likelihood of cancer recurrence in the patient, or the likelihood of response to chemotherapy), which comprises: determining in a sample from the patient the expression of a plurality of test genes comprising at least 2, 3, 4, 5, 6, 7, 8, 9, 10, 12, 14, 15, 16, 18, 20, 25, 30, 35, 40 or more genes selected from BCRGs, TCRGs, HLAGs, or OCPGs (e.g., selected from the genes listed in Tables 1-6b or Immune Panel 1, 2 and/or 3), and determining at least one clinical parameter for the patient (e.g., age, tumor size, node status, tumor stage), and using the expression of said plurality of test genes and the clinical parameter(s) in classifying the cancer (e.g., determining the prognosis of the cancer in the patient, or predicting the cancer outcome, the likelihood of response to chemotherapy, the likelihood of cancer recurrence or probability of post-surgery distant metastasis-free survival). In some embodiments, the BCRGs, TCRGs, HLAGs, and/or OCPGs information and the clinical parameter information are combined to yield a quantitative (e.g., numerical) evaluation or score of the prognosis of the cancer in the patient, or cancer outcome, the likelihood of cancer recurrence or probability of post-surgery distant metastasis-free survival. In some embodiments, the expression level of the genes selected from the BCRGs, TCRGs, HLAGs, and OCPGs and the clinical parameter information are combined with the expression level of the genes selected from CCGs (e.g., genes listed in Table 7) to yield a quantitative evaluation score of the prognosis of the cancer in the patient, or cancer outcome, the likelihood of cancer recurrence or probability of post-surgery distant metastasis-free survival. In some embodiments, the expression level of the genes selected from the BCRGs, TCRGs, HLAGs, and OCPGs and the clinical parameter information are combined with the expression level of the PGR, ABCC5 and/or ESR1 genes to yield a quantitative evaluation score of the prognosis of the cancer in the patient, or cancer outcome, the likelihood of cancer recurrence or probability of post-surgery distant metastasis-free survival.

In one aspect, the present disclosure provides a method for treating cancer, which comprises: determining in a sample from a patient the expression of a plurality of test genes comprising at least 4, 6, 8, 10, 12, or 15 or more BCRGs, TCRGs, HLAGs, or OCPGs (e.g., at least 3 of the genes listed in Tables 1-6b or at least three of the ISGs listed in Table 39), and recommending, prescribing or administering a particular treatment regimen (e.g., a treatment regimen comprising chemotherapy) based at least in part on the determined expression levels of said BCRGs, TCRGs, HLAGs, or OCPGs. In some embodiments, a treatment regimen comprising chemotherapy is recommended, prescribed or administered based at least in part on the expression levels of said BCRGs, TCRGs, HLAGs, or bpOCPGs. In some embodiments, a treatment regimen comprising surgical resection or radiation is recommended prescribed or administered in addition to chemotherapy based at least in part on the expression levels of said BCRGs, TCRGs, HLAGs, or bpOCPGs. In some embodiments, a treatment regimen comprising surgical resection or radiation is not recommended prescribed or administered in addition to chemotherapy based at least in part on the expression levels of said BCRGs, TCRGs, HLAGs, or bpOCPGs. In some embodiments, a treatment regimen comprising chemotherapy is recommended, prescribed or administered based at least in part on the determination that the sample has low (or not increased) expression of said BCRGs, TCRGs, HLAGs, or bpOCPGs. In some embodiments, a treatment regimen comprising hormonal therapy is recommended, prescribed or administered based at least in part on the determination that the sample has high (or increased) expression of said BCRGs, TCRGs, HLAGs, or bpOCPGs.

In one aspect, the present disclosure provides a method for treating cancer, which comprises: determining in a sample from a patient the expression of a plurality of test genes comprising at least 4, 6, 8, 10, 12, or 15 or more BCRGs, TCRGs, HLAGs, or OCPGs (e.g., at least 3 of the genes listed in Tables 1-6b or at least three of the ISGs listed in Table 39), and at least 4, 6, 8, 10, 12, or 15 or more cell cycle genes (e.g., at least 3 of the genes listed in Table 7), and recommending, prescribing or administering a particular treatment regimen (e.g., a treatment regimen comprising chemotherapy) based at least in part on the determined expression levels of said BCRGs, TCRGs, HLAGs, or OCPGs, and said cell cycle genes. In some embodiments, a treatment regimen comprising chemotherapy is recommended, prescribed or administered based at least in part on the expression levels of said BCRGs, TCRGs, HLAGs, or OCPGs, and said cell cycle genes. In some embodiments, a treatment regimen comprising surgical resection or radiation is recommended prescribed or administered in addition to chemotherapy based at least in part on the expression levels of said BCRGs, TCRGs, HLAGs, or OCPGs, and said cell cycle genes. In some embodiments, a treatment regimen comprising surgical resection or radiation is not recommended prescribed or administered in addition to chemotherapy based at least in part on the expression levels of said BCRGs, TCRGs, HLAGs, or OCPGs, and said cell cycle genes. In some embodiments, a treatment regimen comprising chemotherapy is recommended, prescribed or administered based at least in part on the determination that the sample has low (or not increased) expression of said BCRGs, TCRGs, HLAGs, or bpOCPGs. In some embodiments, a treatment regimen comprising hormonal therapy is recommended, prescribed or administered based at least in part on the determination that the sample has high (or increased) expression of said BCRGs, TCRGs, HLAGs, or bpOCPGs.

In another aspect, the present disclosure provides a method for treating breast cancer in a patient, which comprises: determining in a sample from the patient the expression of a plurality of test genes comprising at least 4, 6, 8, 10, 12, or 15 or more BCRGs, TCRGs, HLAGs, or bpOCPGs (e.g., at least 3 of the genes listed in Tables 1-6b or at least three of the ISGs listed in Table 39), and determining in the same or a different sample from the patient the expression of the PGR gene, and recommending, prescribing or administering a particular treatment regimen (e.g., a treatment regimen comprising chemotherapy) based at least in part on the determined expression of the plurality of test genes, as well as the determined PGR expression. In some embodiments, a treatment regimen comprising a non-hormonal therapy agent (e.g., chemotherapy) or radiotherapy is recommended, prescribed or administered based at least in part on any of (1) low (or not increased) expression levels of the plurality of test genes or (2) low (or decreased) level of PGR expression. In some embodiments, a treatment regimen comprising hormonal therapy is recommended, prescribed or administered based at least in part on increased level of PGR expression.

In another aspect, the present disclosure provides a method for treating breast cancer in a patient, which comprises: determining in a sample from the patient the expression of a plurality of test genes comprising at least 2, 3, 4, 5, 6, 7, 8, 9, 10, 12, or 15 or more BCRGs, TCRGs, HLAGs, or bpOCPGs (e.g., at least 3 of the genes listed in Tables 1-6b or at least three of the ISGs listed in Table 39), and determining in the same or a different sample from the patient the expression of the PGR gene, and the ABCC5 gene, and recommending, prescribing or administering a particular treatment regimen (e.g., a treatment regimen comprising chemotherapy) based at least in part on the determined expression of the plurality of test genes, as well as the determined PGR, and ABCC5 expression. In some embodiments, a treatment regimen comprising a non-hormonal therapy agent (e.g., chemotherapy) or radiotherapy is recommended, prescribed or administered based at least in part on any of (1) low (or not increased) expression levels of the plurality of test genes or (2) low (or decreased) level of PGR expression or (3) high (or increased) level of ABCC5 expression. In some embodiments, a treatment regimen comprising hormonal therapy is recommended, prescribed or administered based at least in part on increased level of PGR expression and or increased level of ABCC5 expression.

In another aspect, the present disclosure provides a method for treating breast cancer in a patient, which comprises: determining in a sample from the patient the expression of a plurality of test genes comprising at least 2, 3, 4, 5, 6, 7, 8, 9, 10, 12 or 15 or more cell-cycle genes (e.g., at least 3 of the genes listed in Table 7) and at least 2, 3, 4, 5, 6, 7, 8, 9, 10, 12, or 15 or more BCRGs, TCRGs, HLAGs, or OCPGs (e.g., at least 3 of the genes listed in Tables 1-6b or at least three of the ISGs listed in Table 39), and determining in the same or different sample from the patient the expression of the PGR gene, and recommending, prescribing or administering a particular treatment regimen (e.g., a treatment regimen comprising chemotherapy) based at least in part on the determined expression of the plurality of test genes, as well as the determined PGR expression. In some embodiments, a treatment regimen comprising a non-hormonal therapy agent (e.g., chemotherapy) or radiotherapy is recommended, prescribed or administered based at least in part on any one or both of (1) high (or increased) levels of the CCGs or wpOCPGs in the plurality of test genes or (2) low (or decreased) level of PGR expression. In some embodiments, a treatment regimen comprising a non-hormonal therapy agent (e.g., chemotherapy) or radiotherapy, and not comprising hormonal therapy, is recommended, prescribed or administered based at least in part on any one or both of (1) high (or increased) level of the CCGs or wpOCPGs in the plurality of test genes and (2) low (or decreased) level of PGR expression. In some embodiments, a treatment regimen comprising hormonal therapy is recommended, prescribed or administered based at least in part on high (or increased) level of PGR expression.

In some embodiments of the methods described above, the patient is ER+ and node negative. In some embodiments, the patient is ER+ and node negative, has undergone surgery to remove the tumor in her breast, and is placed on hormone therapy. In some embodiments of the methods described above, the patient is ER+ and node positive. In some embodiments, the expression of the ESR1 gene has been determined (e.g., to determine or confirm the breast cancer is ER+ or ER−).

In yet another aspect, the present disclosure provides a method for treating breast cancer in a patient, which comprises: determining in a sample from the patient the expression of a plurality of test genes comprising at least 2, 3, 4, 5, 6, 7, 8, 9, 10, 12 or 15 or more cell-cycle genes (e.g., at least 3 of the genes listed in Table 7) and at least 2, 3, 4, 5, 6, 7, 8, 9, 10, 12, or 15 or more BCRGs, TCRGs, HLAGs, or OCPGs (e.g., at least 3 of the genes listed in Tables 1-6b or at least three of the ISGs listed in Table 39), and determining in the same or different sample from the patient the expression of the PGR gene, and the ABCC5 gene, and recommending, prescribing or administering a particular treatment regimen (e.g., a treatment regimen comprising chemotherapy) based at least in part on the determined expression of the plurality of test genes, as well as the determined PGR, and ABCC5 expression. In some embodiments, a treatment regimen comprising a non-hormonal therapy agent (e.g., chemotherapy) or radiotherapy is recommended, prescribed or administered based at least in part on any one or both of (1) high (or increased) levels of the CCGs or wpOCPGs in the plurality of test genes or (2) low (or decreased) level of PGR expression or (3) high (or increased) level of ABCC5 expression. In some embodiments, a treatment regimen comprising a non-hormonal therapy agent (e.g., chemotherapy) or radiotherapy, and not comprising hormonal therapy, is recommended, prescribed or administered based at least in part on any one or both of (1) high (or increased) level of the CCGs or wpOCPGs in the plurality of test genes and (2) low (or decreased) level of PGR expression and (3) high (or increased) level of ABCC5 expression. In some embodiments, a treatment regimen comprising hormonal therapy is recommended, prescribed or administered based at least in part on high (or increased) level of PGR expression. In some embodiments, a treatment regimen comprising hormonal therapy is recommended, prescribed or administered based at least in part on low (or decreased) level of ABCC5 expression.

In some embodiments of the methods described above, the patient is ER+ and node negative. In some embodiments, the patient is ER+ and node negative, has undergone surgery to remove the tumor in her breast, and is placed on hormone therapy. In some embodiments of the methods described above, the patient is ER+ and node positive.

In some embodiments, the plurality of test genes includes at least 3 genes selected from BCRGs, TCRGs, HLAGs, or OCPGs, or at least 4, 5, 6, 7, 8, 9, 10, 12, 15, 20, 25 or 30 BCRGs, TCRGs, HLAGs, or OCPGs. In some embodiments, all of the test genes are BCRGs, TCRGs, HLAGs, or OCPGs. In some embodiments, the plurality of test genes includes at least 3 BCRGs, TCRGs, HLAGs, or OCPGs, or at least 4, 5, 6, 7, 8, 9, 10, 12, 15, 20, 25 or 30 BCRGs, TCRGs, HLAGs, or OCPGs. In some embodiments, at least 5%, 10%, 15%, 20%, 25%, 30%, 40%, 50%, 60%, 70%, 80%, 85%, 90%, 95%, or 99% of the plurality of test genes are BCRGs, TCRGs, HLAGs, or OCPGs. In some embodiments, in addition to the BCRGs, TCRGs, HLAGs, or OCPGs, the plurality of test genes includes at least 3 cell-cycle genes, or at least 4, 5, 6, 7, 8, 9, 10, 15, 20, 25 or 30 cell cycle genes. In some embodiments, at least 10%, 15%, 20%, 25%, 30%, 40%, 50%, 60%, 70%, 80%, 85%, 90%, 95%, or 99% of the plurality of test genes are cell cycle genes and BCRGs, TCRGs, HLAGs, or OCPGs.

In some embodiments, the step of determining the expression of the plurality of test genes in the sample comprises measuring the amount of mRNA in the sample transcribed from each of 2, 3, 4, 5, 6, 7, 8, 9, 10, 12 or 15 or more BCRGs, TCRGs, HLAGs, or OCPGs (e.g., at least 3 of the genes listed in Tables 1-6b or at least three of the ISGs listed in Table 39); and measuring the amount of mRNA of one or more control (e.g., housekeeping) genes in the sample. In some embodiments, the step of determining the expression of the plurality of test genes in the sample further comprises measuring the amount of mRNA in the sample transcribed from each of 2, 3, 4, 5, 6, 7, 8, 9, 10, 12 or 15 or more cell cycle genes (e.g., at least 3 of the genes listed in Table 7). In one aspect of these embodiments, the mRNA is converted to cDNA. In a more specific aspect, the cDNA is amplified by PCR.

In some embodiments, the step of determining the expression of the plurality of test genes in the sample comprises (1) determining in a sample from a patient having cancer the expression of a panel of genes in said sample including 2, 3, 4, 5, 6, 7, 8, 9, 10, 12 or 15 or more BCRGs, TCRGs, HLAGs, or OCPGs (e.g., at least 3 of the genes listed in Tables 1-6b or at least three of the ISGs listed in Table 39); and (2) providing a “ISG/OCPG score”, “ISG score”, “BCRG score”, “TCRG score”, “HLAG score”, “OCPG score”, “BCRG/OCPG score”, “TCRG/OCPG score”, “HLAG/OCPG score”, “BCRG/TCRG score”, “BCRG/HLGA score”, “TCRG/HLGA score”, “BCRG/TCRG/OCPG score”, “BCRG/HLGA/OCPG score”, or “TCRG/HLGA/OCPG score” (depending on what type of genes were analyzed in step (1)) by (a) weighting the determined expression of each of a plurality of test genes selected from the panel of genes (which may include all genes in the panel) with a predefined coefficient, and (b) combining the weighted expression to provide the score, wherein at least 5%, at least 10%, at least 25%, at least 50%, at least 75% or at least 85% of the plurality of test genes used to derive the score are, depending on what type of score is being derived, ISGs, BCRGs, TCRGs, HLAGs, or OCPGs (or wherein ISGs, BCRGs, TCRGs, HLAGs, or OCPGs represent at least 5%, at least 10%, at least 25%, at least 50%, at least 75% or at least 85% of the combined weight used to provide the score). For example, if an ISG score is being derived, at least 5%, at least 10%, at least 25%, at least 50%, at least 75% or at least 85% of the plurality of test genes used to derive the ISG score are ISGs (and so forth for the other scores). In some embodiments, at least one of the plurality of test genes is chosen from the group consisting of ABCC5, PGR, and ESR1. In some embodiments, the plurality of test genes comprises ABCC5, PGR, and ESR1. In some embodiments the ABCC5, PGR, or ESR1 genes (i.e., any one, all three together, or any combination of the three) represent at least 5%, 10%, 15%, 20%, 25%, 30%, 35%, 40%, 45%, 50% or more of the combined weight used to provide the combined score.

In some embodiments, the step of determining the expression of the plurality of test genes in the sample comprises (1) determining in a sample from a patient having cancer the expression of a panel of genes in said sample including (a) at least 2, 3, 4, 5, 6, 7, 8, 9, 10, 12 or 15 or more cell-cycle genes and (b) at least 2, 3, 4, 5, 6, 7, 8, 9, 10, 12 or 15 or more BCRGs, TCRGs, HLAGs, and OCGPs; and (2) providing a “ISG/OCPG/CCG combined score”, “ISG/CCG combined score”, “BCRG/CCG combined score”, “TCRG/CCG combined score”, “HLAG/CCG combined score”, or “OCPG/CCG combined score”, “OCPG/BCRG/CCG combined score”, “OCPG/TCRG/CCG combined score”, “OCPG/HLAG/CCG combined score”, “BCRG/TCRG/CCG combined score”, “BCRG/HLAG/CCG combined score”, “TCRG/HLAG/CCG combined score”, “OCPG/BCRG/TCRG/CCG combined score”, “OCPG/BCRG/HLAG/CCG combined score”, “OCPG/TCRG/HLAG/CCG combined score” (depending on what type of genes were analyzed in step (1)) by (a) weighting the determined expression of each of a plurality of test genes selected from the panel of genes with a predefined coefficient, and (b) combining the weighted expression to provide the combined score, wherein at least 50%, at least 75% or at least 85% of the plurality of test genes cell-cycle genes and, depending on what type of combined score is being derived, ISGs, BCRGs, TCRGs, HLAGs, or OCGPs (or wherein CCGs, ISGs, BCRGs, TCRGs, HLAGs, or OCGPs represent at least 50%, at least 75% or at least 85% of the combined weight used to provide the combined score). For example, if an ISG/CCG combined score is being derived, ISGs and CCGs make up at least 5%, at least 10%, at least 25%, at least 50%, at least 75% or at least 85% of the plurality of test genes used to derive the ISG/CCG combined score (and so forth for the other combined scores). In some embodiments, at least one of the plurality of test genes is chosen from the group consisting of ABCC5, PGR, and ESR1. In some embodiments, the plurality of test genes comprises ABCC5, PGR, and ESR1. In some embodiments the ABCC5, PGR, or ESR1 genes (i.e., any one, all three together, or any combination of the three) represent at least 5%, 10%, 15%, 20%, 25%, 30%, 35%, 40%, 45%, 50% or more of the combined weight used to provide the combined score.

In one aspect of the present disclosure, a method is provided for determining gene expression in a sample from a patient identified as having cancer (e.g., breast cancer, prostate cancer, lung cancer, bladder cancer, ovarian cancer, colorectal cancer, or brain cancer). Generally, the method includes at least the following steps: (1) obtaining, or providing, a sample from a patient identified as having cancer (e.g., breast cancer, prostate cancer, lung cancer, bladder cancer, ovarian cancer, colorectal cancer, or brain cancer); (2) determining the expression of a panel of genes in said sample including at least 4 cell-cycle genes chosen from the group in Panel H in Table 17 and at least 4 BCRGs, TCRGs, HLAGs, or OCPGs chosen from the group in Table 1 (e.g., Immune Panel 1, 2 or 3); and (3) providing a test value by (a) weighting the determined expression of each of a plurality of test genes selected from said panel of genes with a predefined coefficient, and (b) combining the weighted expression to provide said test value, wherein at least 50%, at least 75% or at least 90% of said plurality of test genes are cell-cycle genes and BCRGs, TCRGs, HLAGs, or OCPGs (or wherein CCGs and ISGs, BCRGs, TCRGs, HLAGs, or OCGPs represent at least 50%, at least 75% or at least 85% of the combined weight used to provide the test value).

In preferred embodiments, the plurality of test genes includes at least 2, 3, 4, 5, 6, 7, 8, 9, 10, 12, 15, 20, or 25 cell-cycle genes from Panel H in Table 17 and at least 2, 3, 4, 5, 6, 7, 8, 9, 10, 12, 15, 20, or 25 BCRGs, TCRGs, HLAGs, or OCPGs from Table 1. In some preferred embodiments, the plurality of test genes consists of (or consists essentially of) cell-cycle genes and BCRGs, TCRGs, HLAGs, or OCPGs.

In another aspect of the present disclosure, a method is provided for determining the prognosis of breast cancer, prostate cancer, lung cancer, bladder cancer or brain cancer, which comprises determining, in a sample from a patient diagnosed of breast cancer, prostate cancer, lung cancer, bladder cancer or brain cancer, the expression of at least 2, 3, 4, 5, 6, 7, 8, 9, 10, 12 or 15 or more cell-cycle genes in Panel H in Table 17 and the expression of at least 2, 3, 4, 5, 6, 7, 8, 9, 10, 12 or 15 or more BCRGs, TCRGs, HLAGs, bpOCPGs or wpOCPGs in Table 1, and correlating high (or increased) expression of said cell-cycle genes and wpOCPGs and/or low (or decreased) expression of said BCRGs, TCRGs, HLAGs, or bpOCPGs to a poor prognosis or an increased likelihood of recurrence of cancer in the patient. In one aspect, the cancer is breast cancer. In some embodiments, the expression of the ESR1 gene has been determined (e.g., to determine or confirm the patient is ER+ or ER−). In one aspect, the breast cancer is ER positive.

In one embodiment, the prognosis method comprises (1) determining in a sample from a patient diagnosed with breast cancer, prostate cancer, lung cancer, bladder cancer or brain cancer, the expression of a panel of genes in said sample including at least 2, 3, 4, 5, 6, 7, 8, 9, 10, 12 or 15 or more cell-cycle genes in Panel H in Table 17 and at least 2, 3, 4, 5, 6, 7, 8, 9, 10, 12 or 15 or more BCRGs, TCRGs, HLAGs or OCPGs in Table 1; (2) providing a test value by (a) weighting the determined expression of each of a plurality of test genes selected from the panel of genes with a predefined coefficient, and (b) combining the weighted expression to provide the test value, wherein at least 50%, at least 75% or at least 85% of the plurality of test genes are cell-cycle genes in Panel H in Table 17 and BCRGs, TCRGs, HLAGs, bpOCPGs, or wpOCPGs in Table 1, and (3) correlating (a) a high (or increased) level of overall expression of the CCGs and wpOCPGs and low (or decreased or not increased) levels of expression of the BCRGs, TCRGs, HLAGs and bpOCPGs to a poor or worse prognosis, or (b) low (or decreased or not increased) overall expression of the CCGs and wpOCPGs test genes to a good or better prognosis (e.g., a low likelihood of recurrence of cancer in the patient or a higher likelihood of distant metastasis free survival), or (c) a high (or increased) level of expression of BCRGs, TCRGs, HLAGs, or bpOCPGs to a good or better prognosis. In one aspect, the cancer is breast cancer. In one aspect, the breast cancer is ER positive. In some embodiments, the expression of the ESR1 gene has been determined (e.g., to determine or confirm the breast cancer is ER+ or ER−). In some embodiments the prognosis includes a predicting response to chemotherapy.

In preferred embodiments, the prognosis method further includes a step of comparing the test value provided in step (2) above to one or more reference values, and correlating the test value to a risk of cancer progression or risk of cancer recurrence. Optionally an increased likelihood of poor or worse prognosis is indicated if the test value is greater than the reference value.

In some embodiments of the disclosure, the plurality of ISGS and/or OCPGs are chosen from Immune Panel 1, 2, and/or 3. In some embodiments, as described in detail throughout this document, ISGs and/or OCPGs are combined with CCGs to form a combined panel. In some of these embodiments the combined panel is Combined Panel 1 (as shown in Table 39), or a subset of 2, 3, 4, 5, 6, 7, 8, 9, 10, 12, 14, 15, 16, 18, 20, 25 or more genes thereof. In some of these embodiments the combined panel is Combined Panel 2 (as shown in Table 40), or a subset of 2, 3, 4, 5, 6, 7, 8, 9, 10, 12, 14, 15, 16, 18, 20, 25 or more genes thereof.

In yet another aspect, the present disclosure also provides a method of treating cancer in a patient identified as having breast cancer, prostate cancer, lung cancer, bladder cancer or brain cancer, comprising: (1) determining in a sample from a patient diagnosed with breast cancer, prostate cancer, lung cancer, bladder cancer or brain cancer, the expression of a panel of genes in the sample including at least 4 or at least 8 cell-cycle genes in Panel H in Table 17 and at least 4 or at least 8 BCRGs, TCRGs, HLAGs, wpOCPGs, or bpOCPGs in Table 1; (2) providing a test value by (a) weighting the determined expression of each of a plurality of test genes selected from said panel of genes with a predefined coefficient, and (b) combining the weighted expression to provide said test value, wherein at least 50% or 75% or 85% of the plurality of test genes are cell-cycle genes and BCRGs, TCRGs, HLAGs, wpOCPGs or bpOCPGs; (3) correlating (a) a high (or increased) level of expression of the CCGs and wpOCPGs to a poor prognosis, or (b) a low (or decreased or not increased) level of expression of the CCGs and wpOCPGs to a good or better prognosis, or (c) a high (or increased) level of expression of BCRGs, TCRGs, HLAGs, or bpOCPGs to a good or better prognosis; and (4) recommending, prescribing or administering (a) a treatment regimen based at least in part on the prognosis arrived at in step (3)(a) or (b) watchful waiting based at least in part on the prognosis arrived at in step (3)(b) or step (3)(c). In one aspect, the cancer is breast cancer. In one aspect, the breast cancer in ER positive. In some embodiments, the expression of the ESR1 gene has been determined (e.g., to determine or confirm the breast cancer is ER+ or ER−). In some embodiments the prognosis includes a predicting response to chemotherapy.

The present disclosure further provides a diagnostic kit for determining the prognosis of a cancer in a patient, comprising, in a compartmentalized container, a plurality of oligonucleotides hybridizing to at least 3, 4, 5, 6, 7, 8, 9, 10, 12 or 15 or more test genes, wherein less than 10%, 30% or less than 40% of the test genes are not cell-cycle genes, BCRGs, TCRGs, HLAGs, or OCPGs. Optionally but not necessarily, the kit further includes one or more oligonucleotides hybridizing to the PGR, ABCC5, or ESR1 gene. The kit may further include one or more oligonucleotides hybridizing to at least one control (e.g., housekeeping) gene. The oligonucleotides can be hybridizing probes for hybridization with an amplification product of the gene(s) (e.g., an amplification product of an mRNA or cDNA corresponding to the gene) under stringent conditions or primers suitable for PCR amplification of the genes (e.g., suitable for amplification of an mRNA, or corresponding cDNA, of a sample obtained from, e.g., fresh tumor tissue or FFPE tumor tissue). In one embodiment, the kit consists essentially of, in a compartmentalized container, a plurality of PCR reaction mixtures for PCR amplification of mRNA, or corresponding cDNA, from 5 or 10 to about 300 test genes, wherein at least 30% or 50%, at least 60% or at least 80% of such test genes are cell-cycle genes and BCRGs, TCRGs, HLAGs, or OCRGs, and wherein each reaction mixture comprises a PCR primer pair for PCR amplifying an mRNA, or corresponding cDNA, that corresponds to one of the test genes. In some embodiments the kit includes instructions for correlating (a) high (or increased) level of overall expression of the CCGs and wpOCPGs and low (or decreased or not increased), levels of expression of the BCRGs, TCRGs, HLAGs and bpOCPGs to a poor or worse prognosis, or (b) low (or decreased or not increased) overall expression of the CCGs and wpOCPGs test genes to a good or better prognosis (e.g., a low likelihood of recurrence of cancer in the patient or a higher likelihood of distant metastasis free survival). In some embodiments the kit comprises one or more computer software programs for calculating a test value representing the expression of the test genes (either the overall expression of all test genes or of some subset) and for comparing this test value to some reference value. In some embodiments such computer software is programmed to weight the test genes such that the cell-cycle genes and BCRGs, TCRGs, HLAGs, or OCRGs are weighted to contribute at least 50%, at least 75% or at least 85% of the test value. In some embodiments such computer software is programmed to communicate (e.g., display) a particular cancer classification (e.g., that the patient has a particular prognosis, such as an increased likelihood of response to a treatment regimen comprising chemotherapy if the test value is greater than the reference value (e.g., by more than some predetermined amount)). In one aspect, the kit includes reagents necessary for extracting mRNA from fresh tumor tissue, fresh frozen tumor tissue, or FFPE tumor tissue.

The present disclosure also provides the use of (1) a plurality of oligonucleotides hybridizing to mRNAs, or corresponding cDNAs, corresponding to at least 2, 3, 4, 5, 6, 7, 8, 9, 10, 12 or 15 or more cell-cycle genes and a plurality of oligonucleotides hybridizing to mRNAs, or corresponding cDNAs, corresponding to at least 2, 3, 4, 5, 6, 7, 8, 9, 10, 12 or 15 or more genes selected from BCRGs, TCRGs, HLAGs, or OCPGs; optionally (2) one or more oligonucleotides hybridizing to an mRNA, or corresponding cDNA, corresponding to the PGR, ABCC5, or ESR1 gene, for determining the expression of the test genes in a sample from a patient having cancer, for the prognosis of cancer in the patient, wherein an increased level of the overall expression of the test genes indicates an increased likelihood, whereas no increase in the overall expression of the test genes indicates no increased likelihood. In some embodiments, the oligonucleotides are PCR primers suitable for PCR amplification of the test genes. In other embodiments, the oligonucleotides are probes hybridizing to mRNAs, or corresponding cDNAs, that correspond to the test genes under stringent conditions. In some embodiments, the plurality of oligonucleotides are probes for hybridization under stringent conditions to, or are suitable for PCR amplification of mRNAs, or corresponding cDNAs, that correspond to from 4 to about 300 test genes, at least 50%, 70% or 80% or 90% of the test genes being cell-cycle genes and BCRGs, TCRGs, HLAGs, or OCPGs. In some other embodiments, the plurality of oligonucleotides are hybridization probes for, or are suitable for PCR amplification of, mRNAs, or corresponding cDNAs, of from 20 to about 300 test genes, at least 30%, 40%, 50%, 70% or 80% or 90% of the test genes being cell-cycle genes and BCRGs, TCRGs, HLAGs, or OCPGs.

The present disclosure further provides a system for classifying cancer in a patient, comprising: (1) a sample analyzer for determining the expression levels of a panel of genes in a sample including the expression levels of at least 2, 3, 4, 5, 6, 7, 8, 9, 10, 12 or 15 or more test genes selected from BCRGs, TCRGs, HLAGs, or OCPGs, and optionally the ABCC5, PGR, or ESR1 gene (i.e., any one, all three, or any combination of the three), wherein the sample analyzer contains the sample, mRNA molecules expressed from the panel of genes and extracted from the sample, or cDNA molecules corresponding to said mRNA molecules; (2) a first computer program for (a) receiving gene expression data on the test genes, (b) weighting the determined expression of each of the test genes with a predefined coefficient, and (c) combining the weighted expression to provide a test value, wherein at least 5%, at least 10%, at least 25%, at least 50%, at least 75% of the test genes are selected from BCRGs, TCRGs, HLAGs, or OCRGs and optionally the ABCC5, PGR, or ESR1 gene (i.e., any one, all three, or any combination of the three) (or wherein BCRGs, TCRGs, HLAGs, or OCGPs, and optionally the ABCC5, PGR, or ESR1 gene (any one, all three, or any combination of the three), represent at least 50%, at least 75% or at least 85% of the combined weight used to provide the test value); and (3) a second computer program for comparing the test value to one or more reference values each associated with a particular cancer classification (e.g., a predetermined likelihood of cancer recurrence or post-surgery distant metastasis-free survival). In some embodiments, the system further comprises a display module displaying the comparison between the test value and the one or more reference values, or displaying a result of the comparing step. In some embodiments, the system provided determines breast cancer prognosis in a patient.

The present disclosure further provides a system for classifying cancer in a patient, comprising: (1) a sample analyzer for determining the expression levels of a panel of genes in a sample including test genes comprising at least 2, 3, 4, 5, 6, 7, 8, 9, 10, 12 or 15 or more cell-cycle genes, and the expression levels of at least 2, 3, 4, 5, 6, 7, 8, 9, 10, 12 or 15 or more BCRGs, TCRGs, HLAGs, or OCPGs, and optionally the ABCC5, PGR, or ESR1 gene (any one, all three, or any combination of the three), wherein the sample analyzer contains the sample, mRNA molecules expressed from the panel of genes and extracted from the sample, or cDNA molecules corresponding to said mRNA molecules; (2) a first computer program for (a) receiving gene expression data on the test genes, (b) weighting the determined expression of each of the test genes with a predefined coefficient, and (c) combining the weighted expression to provide a test value, wherein at least 50%, at least at least 75% of the test genes are selected from cell-cycle genes and BCRGs, TCRGs, HLAGs, or OCRGs, and optionally the ABCC5, PGR, or ESR1 gene (any one, all three, or any combination of the three) (or wherein CCGs and BCRGs, TCRGs, HLAGs, or OCGPs, and optionally the ABCC5, PGR, or ESR1 gene (any one, all three, or any combination of the three), represent at least 50%, at least 75% or at least 85% of the combined weight used to provide the test value); and (3) a second computer program for comparing the test value to one or more reference values each associated with a particular cancer classification (e.g., a predetermined likelihood of cancer recurrence or post-surgery distant metastasis-free survival). In some embodiments, the system further comprises a display module displaying the comparison between the test value and the one or more reference values, or displaying a result of the comparing step. In some embodiments, the system provided determines breast cancer prognosis in a patient.

Unless otherwise defined, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. In case of conflict, the present specification, including definitions, will control. In addition, the materials, methods, and examples are illustrative only and not intended to be limiting.

Other features and advantages of the disclosure will be apparent from the following Detailed Description, and from the Claims.

### DETAILED DESCRIPTION OF THE INVENTION

The present disclosure is based, in part, on the discovery of gene expression signatures related to classifying cancer. Classifying cancer using these gene expression signatures can include prediction of prognosis for survival (e.g., predicting distant metastasis free survival, etc.) treating cancer (including selection of therapeutic treatments or regimens and predicting response to a particular treatment regimen, etc.), and monitoring cancer.

### A. Immune System Genes Useful in the Invention

In particular, a set of genes related to the immune system (herein referred to as “immune system genes” or “ISGs”) and a set of other genes related to cancer prognosis (herein referred to as “other cancer prognostic genes” or “OCPGs”) were identified as a result of these studies as shown in Table 1. Remarkably, these genes have predictive power for classifying (e.g., assessing prognosis of) cancer, and additionally they add significant prediction power when combined with cell-cycle genes (“CCGs” or “CCP genes”). As will be shown in detail throughout this document, individual ISGs or OCPGs (e.g., individual genes in Table 1) and panels of these genes can also be used in the invention.

The genes identified in these studies include immune system genes, or ISGs, that for convenience can further be subdivided into three subgroups based on their general biological characteristics: B-cell related genes (“BCRGs”), T-cell related genes (“TCRGs”) and HLA related genes (“HLAGs”), and other cancer prognosis genes (“OCPGs”). The BCRGs are genes that are typically expressed in B-cells that were found to be expressed in cancer cells from patients and found to have prognostic value in these studies. The TCRGs are genes that are typically expressed in T-cells that were found to be expressed in cancer cells from patients and found to have prognostic value in these studies. The HLAGs are genes that are typically related to HLA class II activation that were found to be expressed in cancer cells from patients and found to have prognostic value in these studies. These genes are very useful for classifying cancer (e.g., predicting recurrence or distant metastasis free survival in) patients. As described in more detail below, sets of genes selected from the BCRGs, TCRGs, and HLAGs when added to each other, or added to other gene expression profiles such as the CCG expression profiles or the OCPGs, yield exquisitely predictive signatures for cancer prognosis.

TABLE 1
Genes Whose Corresponding Expression Level Is Predictive
of Cancer Prognosis & Corresponding Probes

| Gene | Probeset | Probeset | Gene | Entrez | Representative | RefSeq |
|---|---|---|---|---|---|---|
| # | ID* | ID* | Symbol | Gene ID | Public ID | Transcript ID |
| 1 | 1405_i_at | 1405_i_at | CCL5 | 6352 | M21121 | NM_002985 |
| 2 | 200704_at | 200704_at | LITAF | 9516 | AB034747 | NM_001136472 |
| | | | | | | NM_001136473 |
| | | | | | | NM_004862 |
| | | | | | | NR_024320 |
| 3 | 200706_s_at | 200706_s_at | LITAF | 9516 | NM_004862 | NM_001136472 |
| | | | | | | NM_001136473 |
| | | | | | | NM_004862 |
| | | | | | | NR_024320 |
| 4 | 200904_at | 200904_at | HLA-E | 3133 | X56841 | NM_005516 |
| 5 | 200937_s_at | 200937_s_at | RPL5 | 6125 | NM_000969 | NM_000969 |
| | | | | | | NM_002121 |
| 6 | 201137_s_at | 201137_s_at | HLA-DPB1 | 3115 | NM_002121 | XM_003119096 |
| | | | | | | XM_003119097 |
| | | | | | | XM_003119098 |
| | | | | | | XM_003119099 |
| | | | | | | XM_003119100 |
| | | | | | | XM_003119101 |
| | | | | | | XR_113857 |
| 7 | 201216_at | 201216_at | ERP29 | 10961 | NM_006817 | NM_001034025 |
| | | | | | | NM_006817 |
| 8 | 201225_s_at | 201225_s_at | SRRM1 | 10250 | NM_005839 | NM_005839 |
| 9 | 201368_at | 201368_at | ZFP36L2 | 678 | U07802 | NM_006887 |
| 10 | 201369_s_at | 201369_s_at | ZFP36L2 | 678 | NM_006887 | NM_006887 |
| 11 | 201690_s_at | 201690_s_at | TPD52 | 7163 | AA524023 | NM_001025252 |
| | | | | | | NM_001025253 |
| | | | | | | NM_005079 |
| 12 | 201718_s_at | 201718_s_at | EPB41L2 | 2037 | BF511685 | NM_001135554 |
| | | | | | | NM_001135555 |
| | | | | | | NM_001431 |
| 13 | 201756_at | 201756_at | RPA2 | 6118 | NM_002946 | NM_002946 |
| 14 | 202066_at | 202066_at | PPFIA1 | 8500 | AA195259 | NM_003626 |
| | | | | | | NM_177423 |
| 15 | 202531_at | 202531_at | IRF1 | 3659 | NM_002198 | NM_002198 |
| 16 | 202803_s_at | 202803_s_at | ITGB2 | 3689 | NM_000211 | NM_000211 |
| | | | | | | NM_001127491 |
| 17 | 202957_at | 202957_at | HCLS1 | 3059 | NM_005335 | NM_005335 |
| 18 | 203010_at | 203010_at | STAT5A | 6776 | NM_003152 | NM_003152 |
| 19 | 203108_at | 203108_at | GPRC5A | 9052 | NM_003979 | NM_003979 |
| 20 | 203225_s_at | 203225_s_at | RFK | 55312 | NM_018339 | NM_018339 |
| 21 | 203492_x_at | 203492_x_at | CEP57 | 9702 | AA918224 | NM_014679 |
| 22 | 203493_s_at | 203493_s_at | CEP57 | 9702 | AL525206 | NM_014679 |
| 23 | 203528_at | 203528_at | SEMA4D | 10507 | NM_006378 | NM_001142287 |
| | | | | | | NM_006378 |
| 24 | 203634_s_at | 203634_s_at | CPT1A | 1374 | NM_001876 | NM_001031847 |
| | | | | | | NM_001876 |
| 25 | 204562_at | 204562_at | IRF4 | 3662 | NM_002460 | NM_001195286 |
| | | | | | | NM_002460 |
| | | | | | | NR_036585 |
| 26 | 204563_at | 204563_at | SELL | 6402 | NM_000655 | NM_000655 |
| | | | | | | NR_029467 |
| 27 | 204670_x_at | 204670_x_at | HLA-DRB1 | 3123 | NM_002125 | NM_002124 |
| | | | HLA-DRB4 | 3126 | | NM_021983 |
| 28 | 205404_at | 205404_at | HSD11B1 | 3290 | NM_005525 | NM_005525 |
| | | | | | | NM_181755 |
| 29 | 205656_at | 205656_at | PCDH17 | 27253 | NM_014459 | NM_001040429 |
| 30 | 205692_s_at | 205692_s_at | CD38 | 952 | NM_001775 | NM_001775 |
| 31 | 205817_at | 205817_at | SIX1 | 6495 | NM_005982 | NM_005982 |
| 32 | 206060_s_at | 206060_s_at | PTPN22 | 26191 | NM_015967 | NM_001193431 |
| | | | | | | NM_012411 |
| | | | | | | NM_015967 |
| 33 | 206511_s_at | 206511_s_at | SIX2 | 10736 | NM_016932 | NM_016932 |
| 34 | 206978_at | 206978_at | CCR2 | 729230 | NM_000647 | NM_001123041 |
| | | | | | | NM_001123396 |
| 35 | 207056_s_at | 207056_s_at | SLC4A8 | 9498 | NM_004858 | NM_001039960 |
| | | | | | | NM_004858 |
| 36 | 207238_s_at | 207238_s_at | PTPRC | 5788 | NM_002838 | NM_002838 |
| | | | | | | NM_080921 |
| | | | | | | NM_080923 |
| 37 | 207419_s_at | 207419_s_at | RAC2 | 5880 | NM_002872 | NM_002872 |
| 38 | 208306_x_at | 208306_x_at | HLA-DRB1 | 3123 | NM_021983 | NM_002124 |
| 39 | 208459_s_at | 208459_s_at | XPO7 | 23039 | NM_015024 | NM_015024 |
| 40 | 208894_at | 208894_at | HLA-DRA | 3122 | M60334 | NM_019111 |
| 41 | 208983_s_at | 208983_s_at | PECAM1 | 5175 | M37780 | NM_000442 |
| 42 | 209138_x_at | 209138_x_at | IGL@ | 3535 | M87790 | — |
| 43 | 209302_at | 209302_at | POLR2H | 5437 | U37689 | NM_006232 |
| 44 | 209312_x_at | 209312_x_at | HLA-DRB1 | 3123 | U65585 | NM_002124 |
| | | | HLA-DRB4 | 3126 | | NM_002125 |
| | | | HLA-DRB5 | 3127 | | NM_021983 |
| 45 | 209374_s_at | 209374_s_at | IGHM | 3507 | BC001872 | — |
| 46 | 209380_s_at | 209380_s_at | ABCC5 | 10057 | AF146074 | NM_001023587 |
| | | | | | | NM_005688 |
| 47 | 209619_at | 209619_at | CD74 | 972 | K01144 | NM_001025158 |
| | | | | | | NM_001025159 |
| | | | | | | NM_004355 |
| 48 | 209687_at | 209687_at | CXCL12 | 6387 | U19495 | NM_000609 |
| | | | | | | NM_001033886 |
| | | | | | | NM_001178134 |
| | | | | | | NM_199168 |
| 49 | 209862_s_at | 209862_s_at | CEP57 | 9702 | BC001233 | NM_014679 |
| 50 | 210031_at | 210031_at | CD247 | 919 | J04132 | NM_000734 |
| | | | | | | NM_198053 |
| 51 | 210072_at | 210072_at | CCL19 | 6363 | U88321 | NM_006274 |
| 52 | 210982_s_at | 210982_s_at | HLA-DRA | 3122 | M60333 | NM_019111 |
| 53 | 211150_s_at | 211150_s_at | DLAT | 1737 | J03866 | NM_001931 |
| 54 | 211634_x_at | 211634_x_at | IGHM | 100133862; | M24669 | — |
| | | | LOC100133862 | 3507 | | |
| 55 | 211635_x_at | 211635_x_at | IGHA1 | 100133862; | M24670 | — |
| | | | | 28396; | | |
| | | | | 3493 | | |
| | | | IGHA2 | 3494 | | |
| | | | IGHD | 3495 | | |
| | | | IGHG1 | 3500 | | |
| | | | IGHG3 | 3502 | | |
| | | | IGHG4 | 3503 | | |
| | | | IGHM | 3507 | | |
| | | | IGHV4-31 | | | |
| | | | LOC100133862 | | | |
| 56 | 211645_x_at | 211645_x_at | — | — | M85256 | — |
| 57 | 211654_x_at | 211654_x_at | HLA-DQB1 | 3119 | M17565 | NM_002123 |
| 58 | 211742_s_at | 211742_s_at | EVI2B | 2124 | BC005926 | NM_006495 |
| 59 | 211990_at | 211990_at | HLA-DPA1 | 3113 | M27487 | NM_033554 |
| 60 | 211991_s_at | 211991_s_at | HLA-DPA1 | 3113 | M27487 | NM_033554 |
| 61 | 212592_at | 212592_at | IGJ | 3512 | AV733266 | NM_144646 |
| 62 | 212614_at | 212614_at | ARID5B | 84159 | BG285011 | NM_032199 |
| 63 | 212935_at | 212935_at | MCF2L | 23263 | AB002360 | NM_001112732 |
| | | | | | | NM_024979 |
| 64 | 213502_x_at | 213502_x_at | LOC91316 | 91316 | AA398569 | NR_024448 |
| 65 | 213537_at | 213537_at | HLA-DPA1 | 3113 | AI128225 | NM_033554 |
| 66 | 214211_at | 214211_at | FTH1 | 2495 | AA083483 | NM_002032 |
| 67 | 214669_x_at | 214669_x_at | IGKC | 3514 | BG485135 | — |
| 68 | 214677_x_at | 214677_x_at | IGLV1-44 | 100290481; | X57812 | XM_002348112 |
| | | | LOC100290481 | 28823 | | |
| 69 | 214768_x_at | 214768_x_at | IGKV1-5 | 28299 | BG540628 | — |
| 70 | 214782_at | 214782_at | CTTN | 2017 | AU155105 | NM_001184740 |
| | | | | | | NM_005231 |
| | | | | | | NM_138565 |
| 71 | 214836_x_at | 214836_x_at | IGK@ | 28299 | BG536224 | — |
| | | | IGKC | 3514; | | |
| | | | IGKV1-5 | 50802 | | |
| 72 | 214995_s_at | 214995_s_at | APOBEC3F | 200316; | BF508948 | NM_001006666 |
| | | | APOBEC3G | 60489 | | NM_021822 |
| | | | | | | NM_145298 |
| 73 | 215121_x_at | 215121_x_at | IGLC7 | 100290481; | AA680302 | XM_002348112 |
| | | | IGLV1-44 | 28823; | | |
| | | | LOC100290481 | 28834 | | |
| 74 | 215176_x_at | 215176_x_at | IGK@ | 3514; | AW404894 | — |
| | | | IGKC | 50802 | | |
| 75 | 215193_x_at | 215193_x_at | HLA-DRB1 | 3123 | AJ297586 | NM_002124 |
| | | | HLA-DRB3 | 3125 | | NM_021983 |
| | | | HLA-DRB4 | 3126 | | NM_022555 |
| 76 | 215199_at | 215199_at | CALD1 | 800 | AU147402 | NM_004342 |
| | | | | | | NM_033138 |
| | | | | | | NM_033139 |
| | | | | | | NM_033140 |
| | | | | | | NM_033157 |
| 77 | 215228_at | 215228_at | NHLH2 | 4808 | AA166895 | NM_001111061 |
| | | | | | | NM_005599 |
| 78 | 215379_x_at | 215379_x_at | IGLC7 | 28823; | AV698647 | — |
| | | | IGLV1-44 | 28834 | | |
| 79 | 215946_x_at | 215946_x_at | IGLL3P | 91353 | AL022324 | NR_029395 |
| 80 | 216061_x_at | 216061_x_at | PDGFB | 5155 | AU150748 | NM_002608 |
| | | | | | | NM_033016 |
| 81 | 216191_s_at | 216191_s_at | TRDV3 | 28516 | X72501 | — |
| 82 | 216401_x_at | 216401_x_at | — | — | AJ408433 | — |
| 83 | 216576_x_at | 216576_x_at | IGK@ | 3514; | AF103529 | XM_942302 |
| | | | IGKC | 50802; | | |
| | | | LOC652493 | 652493; | | |
| | | | LOC652694 | 652694 | | |
| 84 | 217022_s_at | 217022_s_at | IGHA1 | 100126583; | S55735 | XR_111480 |
| | | | | 3493 | | XR_114797 |
| | | | IGHA2 | 3494 | | |
| | | | LOC100126583 | | | |
| 85 | 217148_x_at | 217148_x_at | — | — | AJ249377 | — |
| 86 | 217235_x_at | 217235_x_at | IGLL5 | 100423062; | D84140 | NM_001178126 |
| | | | IGLV2-11 | 28816 | | |
| 87 | 217478_s_at | 217478_s_at | HLA-DMA | 3108 | X76775 | NM_006120 |
| 88 | 217767_at | 217767_at | C3 | 718 | NM_000064 | NM_000064 |
| 89 | 218326_s_at | 218326_s_at | LGR4 | 55366 | NM_018490 | NM_018490 |
| 90 | 218379_at | 218379_at | RBM7 | 10179 | NM_016090 | NM_016090 |
| 91 | 218988_at | 218988_at | SLC35E3 | 55508 | NM_018656 | NM_018656 |
| 92 | 219656_at | 219656_at | PCDH12 | 51294 | NM_016580 | NM_016580 |
| 93 | 220731_s_at | 220731_s_at | NECAP2 | 55707 | NM_018090 | NM_001145277 |
| | | | | | | NM_001145278 |
| | | | | | | NM_018090 |
| 94 | 221651_x_at | 221651_x_at | IGK@ | 3514; | BC005332 | — |
| | | | IGKC | 50802 | | |
| 95 | 221671_x_at | 221671_x_at | IGK@ | 3514; | M63438 | — |
| | | | IGKC | 50802 | | |
| 96 | 222020_s_at | 222020_s_at | NTM | 50863 | AW117456 | NM_001048209 |
| | | | | | | NM_001144058 |
| | | | | | | NM_001144059 |
| | | | | | | NM_016522 |
| 97 | 222077_s_at | 222077_s_at | RACGAP1 | 29127 | AU153848 | NM_001126103 |
| | | | | | | NM_001126104 |
| | | | | | | NM_013277 |
| 98 | 222182_s_at | 222182_s_at | CNOT2 | 4848 | BG105204 | NM_014515 |
| 99 | 34726_at | 34726_at | CACNB3 | 784 | U07139 | NM_000725 |
| 100 | 64899_at | 64899_at | LPPR2 | 64748 | AA209463 | NM_001170635 |
| | | | | | | NM_022737 |

*Affymetrix Human Genome U133A or Human Genome U133 Plus 2.0 micro arrays (Santa Clara, CA).

Table 1 above provides a representative set of BCRGs, TCRGs, HLAGs, and OCPGs from which the panels or prognostic signatures of the disclosure as described in the various embodiments and aspects of the disclosure can be constructed. Furthermore, representative probes and identifying information is given in Table 1 from which appropriate probes and/or primer pairs can be designed (or selected) for use in the methods and compositions of the disclosure as described herein. One set of preferred primer pairs and probes for use in the invention correspond to the specific probes (Probeset ID) as described in Table 1 and primers for amplifying an mRNA, or corresponding cDNA, that corresponds to the probe (e.g., binds specifically to the probe).

As used herein, “B-cell related gene(s)” and “BCRG(s)” refer to gene(s) that are characteristically expressed by B-cells, including those listed in Table 2. Table 2 also describes probes that are useful for detecting the expression of these genes. These BCRGs are very useful for classifying cancer. As described in more detail below sets of genes selected from the BCRGs alone, or when added to other gene expression profiles such as TCRGs, HLAGs, OCPGs or cell cycle gene profiles, yield exquisitely predictive signatures for cancer classification. Non-limiting BCRGs are CKAP2, GUSBP11, IGHM, IGJ, IGkappa, IGKC, IGKV1-5, IGL1, IGLL3P, and IGVH.

TABLE 2
B-Cell Related Genes & Probes

| | | Prognosis Associated |
|---|---|---|
| | | with Higher or |
| Probeset ID* | Gene Symbol | Increased Expression |
| 216576_x_at | IGKC | Better |
| 217022_s_at | IGHA1/IGHA2 | Better |
| 217148_x_at | CKAP2 | Better |
| 213502_x_at | GUSBP11 | Better |
| 209374_s_at | IGHM | Better |
| 212592_at | IGJ | Better |
| 214836_x_at | IGkappa | Better |
| 211645_x_at | IGkappa | Better |
| 215176_x_at | IGkappa | Better |
| 216401_x_at | IGkappa | Better |
| 221651_x_at | IGkappa | Better |
| 221671_x_at | IGkappa | Better |
| 214669_x_at | IGKC | Better |
| 214768_x_at | IGKV1-5 | Better |
| 209138_x_at | IGL1 | Better |
| 214677_x_at | IGL1 | Better |
| 215121_x_at | IGL1 | Better |
| 215379_x_at | IGL1 | Better |
| 217235_x_at | IGL1 | Better |
| 215946_x_at | IGLL3P | Better |
| 211634_x_at | IGVH | Better |
| 211635_x_at | IGVH | Better |

*Affymetrix Human Genome U133A or Human Genome U133 Plus 2.0 microarrays (Santa Clara, CA).

As used herein, “T-cell related gene(s)” and “TCRG(s)” refer to gene(s) that are characteristically expressed by T-cells, including those listed in Table 3. Table 3 also describes probes that are useful for detecting the expression of these genes. These TCRGs are very useful for classifying cancer. As described in more detail below sets of genes selected from the BCRGs alone, or when added to other gene expression profiles such as BCRGs, HLAGs, OCPGs or cell cycle gene profiles, yield exquisitely predictive signatures for cancer classification. Non-limiting TCRGs are CCL19, CCL5, CCR2, CD247, CD38, HLA-E, IRF1, IRF4, PTPN22, SELL, SEMA4D, and TCRA/D.

TABLE 3
T-Cell Related Genes

| | | Prognosis Associated |
|---|---|---|
| | | with Higher or |
| Probeset ID* | Gene Symbol | Increased Expression |
| 210072_at | CCL19 | Better |
| 1405_i_at | CCL5 | Better |
| 206978_at | CCR2 | Better |
| 210031_at | CD247 | Better |
| 205692_s_at | CD38 | Better |
| 200904_at | HLA-E | Better |
| 202531_at | IRF1 | Better |
| 204562_at | IRF4 | Better |
| 206060_s_at | PTPN22 | Better |
| 204563_at | SELL | Better |
| 203528_at | SEMA4D | Better |
| 216191_s_at | TCRA/D | Better |

*Affymetrix Human Genome U133A or Human Genome U133 Plus 2.0 microarrays (Santa Clara, CA).

As used herein, “HLA class II activation gene(s)” and “HLAG(s)” refer to gene(s) that are characteristically expressed by cells during HLA class II activation, including those listed in Table 4. Table 4 also describes probes that are useful for detecting the expression of these genes. These HLAGs are very useful for classifying cancer. As described in more detail below sets of genes selected from the BCRGs alone, or when added to other gene expression profiles such as BCRGs, TCRGs, OCPGs or cell cycle gene profiles, yield exquisitely predictive signatures for cancer classification. Non-limiting examples of HLAGs are CD74, EVI2B, HCLS1, HLA-DMA, HLA-DPA1, HLA-DPB1, HLA-DQB1, HLA-DRA, HLA-DRB1, HLA-DRB1/3, ITGB2, PECAM1, and PTPRC.

TABLE 4
HLA Class II Activation Related Genes

| | | Prognosis Associated |
|---|---|---|
| | | with Higher or |
| Probeset ID* | Gene Symbol | Increased Expression |
| 209619_at | CD74 | Better |
| 211742_s_at | EVI2B | Better |
| 202957_at | HCLS1 | Better |
| 217478_s_at | HLA-DMA | Better |
| 211990_at | HLA-DPA1 | Better |
| 211991_s_at | HLA-DPA1 | Better |
| 213537_at | HLA-DPA1 | Better |
| 201137_s_at | HLA-DPB1 | Better |
| 211654_x_at | HLA-DQB1 | Better |
| 208894_at | HLA-DRA | Better |
| 210982_s_at | HLA-DRA | Better |
| 208306_x_at | HLA-DRB1 | Better |
| 204670_x_at | HLA-DRB1/3 | Better |
| 209312_x_at | HLA-DRB1/3 | Better |
| 215193_x_at | HLA-DRB1/3 | Better |
| 202803_s_at | ITGB2 | Better |
| 208983_s_at | PECAM1 | Better |
| 207238_s_at | PTPRC | Better |

*Affymetrix Human Genome U133A or Human Genome U133 Plus 2.0 micro arrays (Santa Clara, CA).

### B. Other Cancer Prognosis Genes Useful in the Invention

As used herein, “Other Cancer Prognosis Gene(s)” and “OCPG(s)” refer to gene(s) identified in these studies that have predictive power in the prognosis of cancer and are characteristic of other pathways in the cell (i.e., not characteristic of B-cells, T-cells, or HLA class II activation), including those listed in Table 5. The OCPGs can be divided into two groups: OCPGs whose higher or increased expression in cancer is associated with good or better prognosis (referred to herein as “better prognosis OCPGs” or “bpOCPGs”), and OCPGs whose higher or increased expression is associated with worse or bad prognosis (referred to herein as “worse prognosis OCPGs” or “wpOCPGs”). Conversely, lower or not increased expression of one or more bpOCPGs is associated with bad or worse prognosis whereas lower or not increased expression of one or more wpOCPGs is associated with good or better prognosis. Table 5 also describes probes useful for detecting and measuring OCPGs. These OCPGs are very useful for classifying cancer. As described in more detail below sets of genes selected from the OCRGs alone, or when added to other gene expression profiles such as BCRGs, TCRGs, HLAGs, or cell cycle gene profiles, yield exquisitely predictive signatures for cancer classification. Non-limiting examples of OCPGs are ABCC5, APOBEC3F, ARID5B, C3, CACNB3, CALD1, CEP57, CNOT2, CPT1A, CTTN, CXCL12, DLAT, EPB41L2, ERP29, ESR1, FTH1, GPRC5A, HSD11B1, LGR4, LITAF, LPPR2, MCF2L, NECAP2, NHLH2, NTM, PCDH12, PCDH17, PDGFB, PGR, POLR2H, PPFIA1, RAC2, RACGAP1, RBM7, RFK, RPA2, RPL5, SIX1, SIX2, SLC35E3, SLC4A8, SRRM1, STAT5A, TPD52, XPO7, and ZFP36L2. OCPGs of particular interest include ABCC5 and PGR. The ABCC5 gene (Entrez GeneID no. 10057) is also known as “ATP-binding cassette, sub-family C (CFTR/MRP), member 5.” Its expression can be determined by, e.g., using ABI Assay ID Hs00981085_m1. The PGR gene (Entrez GeneID no. 5241) is also known as “progesterone receptor gene” and its expression can be determined by, e.g., using ABI Assay ID Hs00172183_m1.

TABLE 5
Other Cancer Prognosis Genes

| | | Prognosis Associated |
|---|---|---|
| | | with Higher or |
| Probeset ID* | Gene Symbol | Increased Expression |
| 209380_s_at | ABCC5 | Worse |
| 214995_s_at | APOBEC3F | Better |
| 212614_at | ARID5B | Better |
| 217767_at | C3 | Better |
| 34726_at | CACNB3 | Worse |
| 215199_at | CALD1 | Worse |
| 203492_x_at | CEP57 | Better |
| 203493_s_at | CEP57 | Better |
| 209862_s_at | CEP57 | Better |
| 222182_s_at | CNOT2 | Worse |
| 203634_s_at | CPT1A | Worse |
| 214782_at | CTTN | Worse |
| 209687_at | CXCL12 | Better |
| 211150_s_at | DLAT | Better |
| 201718_s_at | EPB41L2 | Better |
| 201216_at | ERP29 | Better |
| 214211_at | FTH1 | Worse |
| 203108_at | GPRC5A | Worse |
| 205404_at | HSD11B1 | Better |
| 218326_s_at | LGR4 | Worse |
| 200704_at | LITAF | Better |
| 200706_s_at | LITAF | Better |
| 64899_at | LPPR2 | Worse |
| 212935_at | MCF2L | Worse |
| 220731_s_at | NECAP2 | Better |
| 215228_at | NHLH2 | Worse |
| 222020_s_at | NTM | Worse |
| 219656_at | PCDH12 | Worse |
| 205656_at | PCDH17 | Worse |
| 216061_x_at | PDGFB | Worse |
| 209302_at | POLR2H | Worse |
| 202066_at | PPFIA1 | Worse |
| 207419_s_at | RAC2 | Better |
| 222077_s_at | RACGAP1 | Worse |
| 218379_at | RBM7 | Better |
| 203225_s_at | RFK | Worse |
| 201756_at | RPA2 | Better |
| 200937_s_at | RPL5 | Better |
| 205817_at | SIX1 | Worse |
| 206511_s_at | SIX2 | Worse |
| 218988_at | SLC35E3 | Worse |
| 207056_s_at | SLC4A8 | Worse |
| 201225_s_at | SRRM1 | Better |
| 203010_at | STAT5A | Better |
| 201690_s_at | TPD52 | Better |
| 208459_s_at | XPO7 | Better |
| 201368_at | ZFP36L2 | Better |
| 201369_s_at | ZFP36L2 | Better |

*Affymetrix Human Genome U133A or Human Genome U133 Plus 2.0 micro arrays (Santa Clara, CA).

TABLE 6A
Top 100 ISGs and OCPGs and Probes by p-
value for Independent Predictive Power

| Probe # | Probeset ID* | Coefficient | p-value | Gene symbol |
|---|---|---|---|---|
| 1 | 209862_s_at | −0.83457 | 1.10E−09 | CEP57 |
| 2 | 200704_at | −0.67071 | 6.23E−09 | LITAF |
| 3 | 201368_at | −0.49408 | 1.13E−08 | ZFP36L2 |
| 4 | 218988_at | 0.584973 | 4.10E−08 | SLC35E3 |
| 5 | 207056_s_at | 0.367248 | 9.59E−08 | SLC4A8 |
| 6 | 209312_x_at | −0.34444 | 1.02E−07 | HLA-DRB1/3 |
| 7 | 215193_x_at | −0.31737 | 1.64E−07 | HLA-DRB1/3 |
| 8 | 203108_at | 0.309684 | 1.78E−07 | GPRC5A |
| 9 | 204670_x_at | −0.37003 | 2.40E−07 | HLA-DRB1/3 |
| 10 | 213537_at | −0.33049 | 4.09E−07 | HLA-DPA1 |
| 11 | 215121_x_at | −0.16884 | 4.36E−07 | IGL1 |
| 12 | 215199_at | 0.814482 | 4.56E−07 | CALD1 |
| 13 | 201137_s_at | −0.33654 | 4.87E−07 | HLA-DPB1 |
| 14 | 200706_s_at | −0.52522 | 6.89E−07 | LITAF |
| 15 | 201216_at | −0.69026 | 8.32E−07 | ERP29 |
| 16 | 215379_x_at | −0.16536 | 8.66E−07 | IGL1 |
| 17 | 203492_x_at | −0.64951 | 8.74E−07 | CEP57 |
| 18 | 222077_s_at | 0.801576 | 9.62E−07 | RACGAP1 |
| 19 | 211991_s_at | −0.27829 | 9.69E−07 | HLA-DPA1 |
| 20 | 215946_x_at | −0.26265 | 1.15E−06 | IGLL3P |
| 21 | 216191_s_at | −0.9512 | 1.16E−06 | TCRA/D |
| 22 | 209138_x_at | −0.14286 | 1.29E−06 | IGL1 |
| 23 | 209374_s_at | −0.17814 | 1.53E−06 | IGHM |
| 24 | 203493_s_at | −0.68201 | 1.90E−06 | CEP57 |
| 25 | 210982_s_at | −0.28802 | 2.08E−06 | HLA-DRA |
| 26 | 209619_at | −0.32608 | 2.27E−06 | CD74 |
| 27 | 217478_s_at | −0.31804 | 2.43E−06 | HLA-DMA |
| 28 | 214677_x_at | −0.13116 | 2.52E−06 | IGL1 |
| 29 | 216061_x_at | 0.905321 | 2.56E−06 | PDGFB |
| 30 | 208306_x_at | −0.35548 | 2.72E−06 | HLA-DRB1 |
| 31 | 217767_at | −0.29295 | 2.82E−06 | C3 |
| 32 | 207419_s_at | −0.59772 | 2.94E−06 | RAC2 |
| 33 | 206978_at | −0.38591 | 2.99E−06 | CCR2 |
| 34 | 203528_at | −0.5284 | 3.33E−06 | SEMA4D |
| 35 | 201718_s_at | −0.56159 | 3.33E−06 | EPB41L2 |
| 36 | 208459_s_at | −0.77338 | 3.34E−06 | XPO7 |
| 37 | 219656_at | 1.108938 | 3.55E−06 | PCDH12 |
| 38 | 201690_s_at | 0.410984 | 3.98E−06 | TPD52 |
| 39 | 214836_x_at | −0.21175 | 4.06E−06 | IGkappa |
| 40 | 212592_at | −0.1585 | 4.10E−06 | IGJ |
| 41 | 209687_at | −0.26231 | 4.69E−06 | CXCL12 |
| 42 | 205656_at | 0.809413 | 4.95E−06 | PCDH17 |
| 43 | 213502_x_at | −0.29127 | 5.14E−06 | GUSBP11 |
| 44 | 203634_s_at | 0.476777 | 5.45E−06 | CPT1A |
| 45 | 216576_x_at | −0.18779 | 5.58E−06 | IGKC |
| 46 | 215176_x_at | −0.13777 | 6.00E−06 | IGkappa |
| 47 | 209380_s_at | 0.491385 | 6.17E−06 | ABCC5 |
| 48 | 211990_at | −0.30466 | 6.93E−06 | HLA-DPA1 |
| 49 | 220731_s_at | −0.85893 | 7.49E−06 | NECAP2 |
| 50 | 202531_at | −0.46354 | 7.68E−06 | IRF1 |
| 51 | 214669_x_at | −0.1669 | 8.18E−06 | IGKC |
| 52 | 200904_at | −0.37472 | 8.22E−06 | HLA-E |
| 53 | 212935_at | 0.485768 | 8.44E−06 | MCF2L |
| 54 | 64899_at | 1.048992 | 9.02E−06 | LPPR2 |
| 55 | 222020_s_at | 0.633395 | 9.09E−06 | NTM |
| 56 | 217022_s_at | −0.12334 | 1.01E−05 | IGHA1 /// IGHA2 |
| 57 | 218326_s_at | 0.297686 | 1.02E−05 | LGR4 |
| 58 | 212614_at | −0.35373 | 1.11E−05 | ARID5B |
| 59 | 214995_s_at | −0.76009 | 1.18E−05 | APOBEC3F |
| 60 | 203225_s_at | 0.548894 | 1.18E−05 | RFK |
| 61 | 206060_s_at | −0.73975 | 1.24E−05 | PTPN22 |
| 62 | 202957_at | −0.36745 | 1.30E−05 | HCLS1 |
| 63 | 214768_x_at | −0.19551 | 1.35E−05 | IGKV1-5 |
| 64 | 221671_x_at | −0.15208 | 1.36E−05 | IGkappa |
| 65 | 207238_s_at | −0.30464 | 1.44E−05 | PTPRC |
| 66 | 201225_s_at | −0.66462 | 1.46E−05 | SRRM1 |
| 67 | 208894_at | −0.28315 | 1.52E−05 | HLA-DRA |
| 68 | 205692_s_at | −0.48521 | 1.53E−05 | CD38 |
| 69 | 204562_at | −0.65526 | 1.53E−05 | IRF4 |
| 70 | 217148_x_at | −0.22667 | 1.68E−05 | CKAP2 |
| 71 | 201369_s_at | −0.31786 | 1.80E−05 | ZFP36L2 |
| 72 | 209302_at | 0.71048 | 1.83E−05 | POLR2H |
| 73 | 215228_at | 0.441817 | 1.84E−05 | NHLH2 |
| 74 | 200937_s_at | −0.5351 | 1.90E−05 | RPL5 |
| 75 | 208983_s_at | −0.3936 | 1.97E−05 | PECAM1 |
| 76 | 222182_s_at | 0.583831 | 1.98E−05 | CNOT2 |
| 77 | 204563_at | −0.32723 | 1.99E−05 | SELL |
| 78 | 34726_at | 0.463486 | 2.09E−05 | CACNB3 |
| 79 | 217235_x_at | −0.29096 | 2.40E−05 | IGL1 |
| 80 | 202803_s_at | −0.31225 | 2.40E−05 | ITGB2 |
| 81 | 205404_at | −0.61756 | 2.43E−05 | HSD11B1 |
| 82 | 210072_at | −0.20089 | 2.44E−05 | CCL19 |
| 83 | 216401_x_at | −0.19071 | 2.46E−05 | IGkappa |
| 84 | 211634_x_at | −0.30565 | 2.51E−05 | IGVH |
| 85 | 205817_at | 0.303015 | 2.51E−05 | SIX1 |
| 86 | 1405_i_at | −0.22759 | 2.57E−05 | CCL5 |
| 87 | 211150_s_at | −0.60892 | 2.59E−05 | DLAT |
| 88 | 211742_s_at | −0.32904 | 2.61E−05 | EVI2B |
| 89 | 211645_x_at | −0.15649 | 2.67E−05 | IGkappa |
| 90 | 203010_at | −0.73762 | 2.74E−05 | STAT5A |
| 91 | 210031_at | −0.46472 | 3.03E−05 | CD247 |
| 92 | 214211_at | 0.436131 | 3.03E−05 | FTH1 |
| 93 | 206511_s_at | 0.655529 | 3.15E−05 | SIX2 |
| 94 | 211635_x_at | −0.35609 | 3.15E−05 | IGVH |
| 95 | 201756_at | −0.65305 | 3.19E−05 | RPA2 |
| 96 | 214782_at | 0.40157 | 3.21E−05 | CTTN |
| 97 | 221651_x_at | −0.14468 | 3.22E−05 | IGkappa |
| 98 | 211654_x_at | −0.26674 | 3.28E−05 | HLA-DQB1 |
| 99 | 202066_at | 0.338161 | 3.31E−05 | PPFIA1 |
| 100 | 218379_at | −0.5151 | 3.57E−05 | RBM7 |

*Affymetrix Human Genome U133A or Human Genome U133 Plus 2.0 micro arrays (Santa Clara, CA).

TABLE 6B
Non-redundant Ranking of Genes in Table 6A

| Gene # | Gene symbol |
|---|---|
| 1 | CEP57 |
| 2 | LITAF |
| 3 | ZFP36L2 |
| 4 | SLC35E3 |
| 5 | SLC4A8 |
| 6 | HLA-DRB1/3 |
| 7 | GPRC5A |
| 8 | HLA-DPA1 |
| 9 | IGL1 |
| 10 | CALD1 |
| 11 | HLA-DPB1 |
| 12 | ERP29 |
| 13 | RACGAP1 |
| 14 | IGLL3P |
| 15 | TCRA/D |
| 16 | IGHM |
| 17 | HLA-DRA |
| 18 | CD74 |
| 19 | HLA-DMA |
| 20 | PDGFB |
| 21 | HLA-DRB1 |
| 22 | C3 |
| 23 | RAC2 |
| 24 | CCR2 |
| 25 | SEMA4D |
| 26 | EPB41L2 |
| 27 | XPO7 |
| 28 | PCDH12 |
| 29 | TPD52 |
| 30 | IGkappa |
| 31 | IGJ |
| 32 | CXCL12 |
| 33 | PCDH17 |
| 34 | GUSBP11 |
| 35 | CPT1A |
| 36 | IGKC |
| 37 | ABCC5 |
| 38 | NECAP2 |
| 39 | IRF1 |
| 40 | IGKC |
| 41 | HLA-E |
| 42 | MCF2L |
| 43 | LPPR2 |
| 44 | NTM |
| 45 | IGHA1/IGHA2 |
| 46 | LGR4 |
| 47 | ARID5B |
| 48 | APOBEC3F |
| 49 | RFK |
| 50 | PTPN22 |
| 51 | HCLS1 |
| 52 | IGKV1-5 |
| 53 | PTPRC |
| 54 | SRRM1 |
| 55 | CD38 |
| 56 | IRF4 |
| 57 | CKAP2 |
| 58 | POLR2H |
| 59 | NHLH2 |
| 60 | RPL5 |
| 61 | PECAM1 |
| 62 | CNOT2 |
| 63 | SELL |
| 64 | CACNB3 |
| 65 | ITGB2 |
| 66 | HSD11B1 |
| 67 | CCL19 |
| 68 | IGVH |
| 69 | SIX1 |
| 70 | CCL5 |
| 71 | DLAT |
| 72 | EVI2B |
| 73 | STAT5A |
| 74 | CD247 |
| 75 | FTH1 |
| 76 | SIX2 |
| 77 | RPA2 |
| 78 | CTTN |
| 79 | HLA-DQB1 |
| 80 | PPFIA1 |
| 81 | RBM7 |

In one aspect of the disclosure, the BCRGs, TCRGs, HLAGs, or OCPGs as described in the various embodiments and aspect herein are selected from those that correspond to probe #1 through 5, 1 through 10, 1 through 15, 1 through 20, 1 through 25, 1 through 30, 1 through 40, 1 through 50, 1 through 55, 1 through 60, 1 through 65, 1 through 70, 1 through 75, 1 through 80, 1 through 85, 1 through 90, 1 through 95, or 1 through 100 of Table 6a. In one aspect of the disclosure, the cDNA corresponding to the BCRGs, TCRGs, HLAGs, or OCPGs as described in the various embodiments and aspects herein hybridize specifically to a probe or probes corresponding to those selected from probe #1 through 5, 1 through 10, 1 through 15, 1 through 20, 1 through 25, 1 through 30, 1 through 40, 1 through 50, 1 through 55, 1 through 60, 1 through 65, 1 through 70, 1 through 75, 1 through 80, 1 through 85, 1 through 90, 1 through 95, or 1 through 100 of Table 6a. In one aspect of the disclosure, the primer pairs capable of amplifying an mRNA, or corresponding cDNA, corresponding to BCRGs, TCRGs, HLAGs, or OCPGs as described in the various embodiments and aspects herein are selected from those capable of amplifying said cDNA or mRNA that is capable of specifically hybridizing to a probe or probes corresponding to those selected from probe #1 through 5, 1 through 10, 1 through 15, 1 through 20, 1 through 25, 1 through 30, 1 through 40, 1 through 50, 1 through 55, 1 through 60, 1 through 65, 1 through 70, 1 through 75, 1 through 80, 1 through 85, 1 through 90, 1 through 95, or 1 through 100 of Table 6a.

### C. Cell-Cycle Genes Useful in the Invention

In one aspect of the disclosure, one or more ISGs or OCPGs are combined with one or more cell-cycle genes into a gene panel useful for classifying cancer. “Cell-cycle gene” and “CCG” herein refer to a gene whose expression level closely tracks the progression of the cell through the cell-cycle. See, e.g., Whitfield et al., MOL. BIOL. CELL (2002) 13:1977-2000. The term “cell-cycle progression” or “CCP” will also be used in this application and will generally be interchangeable with CCG (i.e., a CCP gene is a CCG; a CCP score is a CCG score). More specifically, CCGs show periodic increases and decreases in expression that coincide with certain phases of the cell cycle—e.g., STK15 and PLK show peak expression at G2/M. Id. Often CCGs have clear, recognized cell-cycle related function—e.g., in DNA synthesis or repair, in chromosome condensation, in cell-division, etc. However, some CCGs have expression levels that track the cell-cycle without having an obvious, direct role in the cell-cycle—e.g., UBE2S encodes a ubiquitin-conjugating enzyme, yet its expression closely tracks the cell-cycle. Thus a CCG according to the present disclosure need not have a recognized role in the cell-cycle. Exemplary CCGs are listed in Tables 7, 8, 9, 10, 11, 12, 13, or 14. A fuller discussion of CCGs, including an extensive (though not exhaustive) list of CCGs, can be found in International Application No. PCT/US2010/020397 (pub. no. WO/2010/080933 (see also corresponding U.S. application Ser. No. 13/177,887)) (see, e.g., Table 1 in WO/2010/080933 and International Application No. PCT/US2011/043228 (pub no. WO/2012/006447 (see also related U.S. application Ser. No. 13/178,380)), the contents of which are hereby incorporated by reference in their entirety.

Whether a particular gene is a CCG may be determined by any technique known in the art, including those taught in Whitfield et al., MOL. BIOL. CELL (2002) 13:1977-2000; Whitfield et al., MOL. CELL. BIOL. (2000) 20:4188-4198; WO/2010/080933 (¶ [0039]). All of the CCGs in Table 7 below can together form a panel of CCGs (“Panel A”) useful in the disclosure. As will be shown in detail throughout this document, individual CCGs (e.g., CCGs in Table 7) and subsets of these genes can also be used in the disclosure.

TABLE 7

| | Entrez | | RefSeq Accession |
|---|---|---|---|
| Gene Symbol | GeneID | ABI Assay ID | Nos. |
| APOBEC3B* | 9582 | Hs00358981_m1 | NM_004900.3 |
| ASF1B* | 55723 | Hs00216780_m1 | NM_018154.2 |
| ASPM* | 259266 | Hs00411505_m1 | NM_018136.4 |
| ATAD2* | 29028 | Hs00204205_m1 | NM_014109.3 |
| BIRC5* | 332 | Hs00153353_m1; | NM_001012271.1; |
| | | Hs03043576_m1 | NM_001012270.1; |
| | | | NM_001168.2 |
| BLM* | 641 | Hs00172060_m1 | NM_000057.2 |
| BUB1 | 699 | Hs00177821_m1 | NM_004336.3 |
| BUB1B* | 701 | Hs01084828_m1 | NM_001211.5 |
| C12orf48* | 55010 | Hs00215575_m1 | NM_017915.2 |
| C18orf24* | 220134 | Hs00536843_m1 | NM_145060.3; |
| | | | NM_001039535.2 |
| C1orf135* | 79000 | Hs00225211_m1 | NM_024037.1 |
| C21orf45* | 54069 | Hs00219050_m1 | NM_018944.2 |
| CCDC99* | 54908 | Hs00215019_m1 | NM_017785.4 |
| CCNA2* | 890 | Hs00153138_m1 | NM_001237.3 |
| CCNB1* | 891 | Hs00259126_m1 | NM_031966.2 |
| CCNB2* | 9133 | Hs00270424_m1 | NM_004701.2 |
| CCNE1* | 898 | Hs01026536_m1 | NM_001238.1; |
| | | | NM_057182.1 |
| CDC2* | 983 | Hs00364293_m1 | NM_033379.3; |
| | | | NM_001130829.1; |
| | | | NM_001786.3 |
| CDC20* | 991 | Hs03004916_g1 | NM_001255.2 |
| CDC45L* | 8318 | Hs00185895_m1 | NM_003504.3 |
| CDC6* | 990 | Hs00154374_m1 | NM_001254.3 |
| CDCA3* | 83461 | Hs00229905_m1 | NM_031299.4 |
| CDCA8* | 55143 | Hs00983655_m1 | NM_018101.2 |
| CDKN3* | 1033 | Hs00193192_m1 | NM_001130851.1; |
| | | | NM_005192.3 |
| CDT1* | 81620 | Hs00368864_m1 | NM_030928.3 |
| CENPA | 1058 | Hs00156455_m1 | NM_001042426.1; |
| | | | NM_001809.3 |
| CENPE* | 1062 | Hs00156507_m1 | NM_001813.2 |
| CENPF* | 1063 | Hs00193201_m1 | NM_016343.3 |
| CENPI* | 2491 | Hs00198791_m1 | NM_006733.2 |
| CENPM* | 79019 | Hs00608780_m1 | NM_024053.3 |
| CENPN* | 55839 | Hs00218401_m1 | NM_018455.4; |
| | | | NM_001100624.1; |
| | | | NM_001100625.1 |
| CEP55* | 55165 | Hs00216688_m1 | NM_018131.4; |
| | | | NM_001127182.1 |
| CHEK1* | 1111 | Hs00967506_m1 | NM_001114121.1; |
| | | | NM_001114122.1; |
| | | | NM_001274.4 |
| CKAP2* | 26586 | Hs00217068_m1 | NM_018204.3; |
| | | | NM_001098525.1 |
| CKS1B* | 1163 | Hs01029137_g1 | NM_001826.2 |
| CKS2* | 1164 | Hs01048812_g1 | NM_001827.1 |
| CTPS* | 1503 | Hs01041851_m1 | NM_001905.2 |
| CTSL2* | 1515 | Hs00952036_m1 | NM_001333.2 |
| DBF4* | 10926 | Hs00272696_m1 | NM_006716.3 |
| DDX39* | 10212 | Hs00271794_m1 | NM_005804.2 |
| DLGAP5/ | 9787 | Hs00207323_m1 | NM_014750.3 |
| DLG7* | | | |
| DONSON* | 29980 | Hs00375083_m1 | NM_017613.2 |
| DSN1* | 79980 | Hs00227760_m1 | NM_024918.2 |
| DTL* | 51514 | Hs00978565_m1 | NM_016448.2 |
| E2F8* | 79733 | Hs00226635_m1 | NM_024680.2 |
| ECT2* | 1894 | Hs00216455_m1 | NM_018098.4 |
| ESPL1* | 9700 | Hs00202246_m1 | NM_012291.4 |
| EXO1* | 9156 | Hs00243513_m1 | NM_130398.2; |
| | | | NM_003686.3; |
| | | | NM_006027.3 |
| EZH2* | 2146 | Hs00544830_m1 | NM_152998.1; |
| | | | NM_004456.3 |
| FANCI* | 55215 | Hs00289551_m1 | NM_018193.2; |
| | | | NM_001113378.1 |
| FBXO5* | 26271 | Hs03070834_m1 | NM_001142522.1; |
| | | | NM_012177.3 |
| FOXM1* | 2305 | Hs01073586_m1 | NM_202003.1; |
| | | | NM_202002.1; |
| | | | NM_021953.2 |
| GINS1 * | 9837 | Hs00221421_m1 | NM_021067.3 |
| GMPS* | 8833 | Hs00269500_m1 | NM_003875.2 |
| GPSM2* | 29899 | Hs00203271_m1 | NM_013296.4 |
| GTSE1* | 51512 | Hs00212681_m1 | NM_016426.5 |
| H2AFX* | 3014 | Hs00266783_s1 | NM_002105.2 |
| HMMR* | 3161 | Hs00234864_m1 | NM_001142556.1; |
| | | | NM_001142557.1; |
| | | | NM_012484.2; |
| | | | NM_012485.2 |
| HN1* | 51155 | Hs00602957_m1 | NM_001002033.1; |
| | | | NM_001002032.1; |
| | | | NM_016185.2 |
| KIAA0101* | 9768 | Hs00207134_m1 | NM_014736.4 |
| KIF11* | 3832 | Hs00189698_m1 | NM_004523.3 |
| KIF15* | 56992 | Hs00173349_m1 | NM_020242.2 |
| KIF18A* | 81930 | Hs01015428_m1 | NM_031217.3 |
| KIF20A* | 10112 | Hs00993573_m1 | NM_005733.2 |
| KIF20B/ | 9585 | Hs01027505_m1 | NM_016195.2 |
| MPHOSPH1* | | | |
| KIF23* | 9493 | Hs00370852_m1 | NM_138555.1; |
| | | | NM_004856.4 |
| KIF2C* | 11004 | Hs00199232_m1 | NM_006845.3 |
| KIF4A* | 24137 | Hs01020169_m1 | NM_012310.3 |
| KIFC1* | 3833 | Hs00954801_m1 | NM_002263.3 |
| KPNA2 | 3838 | Hs00818252_g1 | NM_002266.2 |
| LMNB2* | 84823 | Hs00383326_m1 | NM_032737.2 |
| MAD2L1 | 4085 | Hs01554513_g1 | NM_002358.3 |
| MCAM* | 4162 | Hs00174838_m1 | NM_006500.2 |
| MCM10* | 55388 | Hs00960349_m1 | NM_018518.3; |
| | | | NM_182751.1 |
| MCM2* | 4171 | Hs00170472_m1 | NM_004526.2 |
| MCM4* | 4173 | Hs00381539_m1 | NM_005914.2; |
| | | | NM_182746.1 |
| MCM6* | 4175 | Hs00195504_m1 | NM_005915.4 |
| MCM7* | 4176 | Hs01097212_m1 | NM_005916.3; |
| | | | NM_182776.1 |
| MELK | 9833 | Hs00207681_m1 | NM_014791.2 |
| MKI67* | 4288 | Hs00606991_m1 | NM_002417.3 |
| MYBL2* | 4605 | Hs00231158_m1 | NM_002466.2 |
| NCAPD2* | 9918 | Hs00274505_m1 | NM_014865.3 |
| NCAPG* | 64151 | Hs00254617_m1 | NM_022346.3 |
| NCAPG2* | 54892 | Hs00375141_m1 | NM_017760.5 |
| NCAPH* | 23397 | Hs01010752_m1 | NM_015341.3 |
| NDC80* | 10403 | Hs00196101_m1 | NM_006101.2 |
| NEK2* | 4751 | Hs00601227_mH | NM_002497.2 |
| NUSAP1* | 51203 | Hs01006195_m1 | NM_018454.6; |
| | | | NM_001129897.1; |
| | | | NM_016359.3 |
| OIP5* | 11339 | Hs00299079_m1 | NM_007280.1 |
| ORC6L* | 23594 | Hs00204876_m1 | NM_014321.2 |
| PAICS* | 10606 | Hs00272390_m1 | NM_001079524.1; |
| | | | NM_001079525.1; |
| | | | NM_006452.3 |
| PBK* | 55872 | Hs00218544_m1 | NM_018492.2 |
| PCNA* | 5111 | Hs00427214_g1 | NM_182649.1; |
| | | | NM_002592.2 |
| PDSS1* | 23590 | Hs00372008_m1 | NM_014317.3 |
| PLK1* | 5347 | Hs00153444_m1 | NM_005030.3 |
| PLK4* | 10733 | Hs00179514_m1 | NM_014264.3 |
| POLE2* | 5427 | Hs00160277_m1 | NM_002692.2 |
| PRC1* | 9055 | Hs00187740_m1 | NM_199413.1; |
| | | | NM_199414.1; |
| | | | NM_003981.2 |
| PSMA7* | 5688 | Hs00895424_m1 | NM_002792.2 |
| PSRC1* | 84722 | Hs00364137_m1 | NM_032636.6; |
| | | | NM_001005290.2; |
| | | | NM_001032290.1; |
| | | | NM_001032291.1 |
| PTTG1* | 9232 | Hs00851754_u1 | NM_004219.2 |
| RACGAP1* | 29127 | Hs00374747_m1 | NM_013277.3 |
| RAD51* | 5888 | Hs00153418_m1 | NM_133487.2; |
| | | | NM_002875.3 |
| RAD51AP1* | 10635 | Hs01548891_m1 | NM_001130862.1; |
| | | | NM_006479.4 |
| RAD54B* | 25788 | Hs00610716_m1 | NM_012415.2 |
| RAD54L* | 8438 | Hs00269177_m1 | NM_001142548.1; |
| | | | NM_003579.3 |
| RFC2* | 5982 | Hs00945948_m1 | NM_181471.1; |
| | | | NM_002914.3 |
| RFC4* | 5984 | Hs00427469_m1 | NM_181573.2; |
| | | | NM_002916.3 |
| RFC5* | 5985 | Hs00738859_m1 | NM_181578.2; |
| | | | NM_001130112.1; |
| | | | NM_001130113.1; |
| | | | NM_007370.4 |
| RNASEH2A* | 10535 | Hs00197370_m1 | NM_006397.2 |
| RRM2* | 6241 | Hs00357247_g1 | NM_001034.2 |
| SHCBP1 * | 79801 | Hs00226915_m1 | NM_024745.4 |
| SMC2* | 10592 | Hs00197593_m1 | NM_001042550.1; |
| | | | NM_001042551.1; |
| | | | NM_006444.2 |
| SPAG5* | 10615 | Hs00197708_m1 | NM_006461.3 |
| SPC25* | 57405 | Hs00221100_m1 | NM_020675.3 |
| STIL* | 6491 | Hs00161700_m1 | NM_001048166.1; |
| | | | NM_003035.2 |
| STMN1* | 3925 | Hs00606370_m1; | NM_005563.3; |
| | | Hs01033129_m1 | NM_203399.1 |
| TACC3* | 10460 | Hs00170751_m1 | NM_006342.1 |
| TIMELESS* | 8914 | Hs01086966_m1 | NM_003920.2 |
| TK1* | 7083 | Hs01062125_m1 | NM_003258.4 |
| TOP2A* | 7153 | Hs00172214_m1 | NM_001067.2 |
| TPX2* | 22974 | Hs00201616_m1 | NM_012112.4 |
| TRIP13* | 9319 | Hs01020073_m1 | NM_004237.2 |
| TTK* | 7272 | Hs00177412_m1 | NM_003318.3 |
| TUBA1C* | 84790 | Hs00733770_m1 | NM_032704.3 |
| TYMS* | 7298 | Hs00426591_m1 | NM_001071.2 |
| UBE2C | 11065 | Hs00964100_g1 | NM_181799.1; |
| | | | NM_181800.1; |
| | | | NM_181801.1; |
| | | | NM_181802.1; |
| | | | NM_181803.1; |
| | | | NM_007019.2 |
| UBE2S | 27338 | Hs00819350_m1 | NM_014501.2 |
| VRK1* | 7443 | Hs00177470_m1 | NM_003384.2 |
| ZWILCH* | 55055 | Hs01555249_m1 | NM_017975.3; |
| | | | NR_003105.1 |
| ZWINT* | 11130 | Hs00199952_m1 | NM_032997.2; |
| | | | NM_001005413.1; |
| | | | NM_007057.3 |

*124-gene subset of CCGs useful in the disclosure (“Panel B”). ABI Assay ID means the catalogue ID number for the gene expression assay commercially available from Applied Biosystems Inc. (Foster City, CA) for the particular gene.

### D. Methods of Classifying Cancer Using ISGs and/or OCPGs of the Invention

Accordingly, in one aspect, the present disclosure provides a method for classifying cancer in a patient (e.g., determining the patient's prognosis, the likelihood of cancer recurrence in the patient, or response to chemotherapy). Generally, the method comprises: determining in a sample from a patient the expression of at least 4, 8, or 12 test genes selected from BCRGs, TCRGs, HLAGs, and OCPGs (e.g., selected from Tables 1, 2, 3, 4 and/or 5), and using the expression of the test genes in classifying the cancer (e.g., determining the prognosis of the cancer in the patient, predicting the cancer outcome, predicting the response to chemotherapy, the likelihood of cancer recurrence or probability of post-surgery distant metastasis-free survival). Thus, in one aspect the disclosure provides a method for classifying cancer comprising: determining in a sample from a patient the expression of a panel of genes comprising at least 4, 8, or 12 test genes selected from Tables 1, 2, 3, 4 and/or 5, and using the expression of the panel of genes in classifying the cancer. In some embodiments, the method comprises correlating an increased or higher expression level of the genes selected from BCRGs, TCRGs, HLAGs, and bpOCPGs, to a favorable cancer classification (e.g., good or better prognosis, decreased likelihood of cancer recurrence, increased probability of response to chemotherapy, or increased probability of post-surgery distant metastasis-free survival). In some embodiments, the method comprises correlating no increase or lower expression levels of the genes selected from BCRGs, TCRGs, HLAGs, and bpOCPGs, to an unfavorable cancer classification (e.g., a bad or worse prognosis, increased likelihood of cancer recurrence, decreased probability of response to chemotherapy, or decreased probability of post-surgery distant metastasis-free survival). In some embodiments, the method comprises correlating an increased or higher expression level of the wpOCPGs, to an unfavorable cancer classification (e.g., a bad or worse prognosis, increased likelihood of cancer recurrence, decreased probability of response to chemotherapy, or decreased probability of post-surgery distant metastasis-free survival). In some embodiments, the method comprises correlating no increase, or lower expression level of the wpOCPGs, to a favorable cancer classification (e.g., good or better prognosis, decreased likelihood of cancer recurrence, increased probability of response to chemotherapy, or increased probability of post-surgery distant metastasis-free survival).

The present disclosure further provides a method for classifying cancer in a patient which comprises: determining in a sample from a patient the expression of at least 4, 8, or 12 test genes selected from BCRGs, TCRGs, HLAGs, and OCPGs (e.g., selected from Tables 1, 2, 3, 4 and/or 5), and at least 4, 8, or 12 test genes selected from CCGs (e.g., selected from Table 7), and using the expression of the test genes in classifying the cancer (e.g., determining the prognosis of the cancer in the patient, predicting the cancer outcome, predicting response to chemotherapy, the likelihood of cancer recurrence or probability of post-surgery distant metastasis-free survival). Thus, in one aspect the disclosure provides a method for classifying cancer comprising: determining in a sample from a patient the expression of a panel of genes comprising at least 4, 8, or 12 test genes selected from Tables 1, 2, 3, 4 and/or 5 and at least 4, 8, or 12 genes selected from Table 7, and using the expression of the panel of genes in classifying the cancer. In some embodiments, the method comprises correlating an increased or higher expression level of the genes selected from BCRGs, TCRGs, HLAGs, and bpOCPGs, to a favorable cancer classification (e.g., good or better prognosis, decreased likelihood of cancer recurrence, increased probability of response to chemotherapy, or increased probability of post-surgery distant metastasis-free survival). In some embodiments, the method comprises correlating no increase or lower expression levels of the genes selected from BCRGs, TCRGs, HLAGs, and bpOCPGs, to an unfavorable cancer classification (e.g., a bad or worse prognosis, increased likelihood of cancer recurrence, decreased probability of response to chemotherapy, or decreased probability of post-surgery distant metastasis-free survival). In some embodiments, the method comprises correlating an increased or higher expression level of the wpOCPGs and/or the CCGs, to an unfavorable cancer classification (e.g., a bad or worse prognosis, increased likelihood of cancer recurrence, or decreased probability of post-surgery distant metastasis-free survival). In some embodiments, the method comprises correlating no increase, or lower expression level of the wpOCPGs and/or CCGs, to a favorable cancer classification (e.g., good or better prognosis, decreased likelihood of cancer recurrence, or increased probability of post-surgery distant metastasis-free survival).

In some embodiments, at least one of said OCPGs is the PGR gene. Thus, in one aspect the disclosure provides a method for classifying cancer comprising: determining in a sample from a patient the expression of the PGR gene and at least 3 genes selected from BCRGs, TCRGs, HLAGs, or OCPGs and using the expression of the PGR gene and the panel of genes in classifying the cancer. In some embodiments, at least one of said OCPGs is the ABCC5 gene. Thus, in one aspect the disclosure provides a method for classifying cancer comprising: determining in a sample from a patient the expression of the ABCC5 gene and at least 3 genes selected from BCRGs, TCRGs, HLAGs, or OCPGs and using the expression of the ABCC5 gene and the panel of genes in classifying the cancer. In some embodiments, at least two of said OCPGs are the PGR and ABCC5 genes. Thus, in one aspect the disclosure provides a method for classifying cancer comprising: determining in a sample from a patient the expression of the ABCC5 gene, the PGR gene and at least 2 genes selected from BCRGs, TCRGs, HLAGs, or OCPGs and using the expression of the ABCC5 and PGR gene and the panel of genes in classifying the cancer. In some embodiments, at least one of said OCPGs is the ESR1 gene. Thus, in one aspect the disclosure provides a method for classifying cancer comprising: determining in a sample from a patient the expression of the ESR1 gene and at least 3 genes selected from BCRGs, TCRGs, HLAGs, or OCPGs and using the expression of the ESR1 gene and the panel of genes in classifying the cancer.

In a specific aspect, the cancer is lung cancer, bladder cancer, prostate cancer, brain cancer, or breast cancer. In another specific aspect, the cancer is breast cancer. In yet another specific aspect, the cancer is ER positive breast cancer.

Clinical parameters can be combined with the information gained from analysis of BCRGs, TCRGs, HLAGs, or OCPGs. Thus, in yet another aspect, the present disclosure provides a method for classifying cancer in a patient (e.g., determining the patient's prognosis or the likelihood of cancer recurrence in the patient), which comprises: determining in a sample from the patient the expression of a plurality of test genes comprising at least 4, 6, 8, 10 or 15 or more genes selected from BCRGs, TCRGs, HLAGs, or OCPGs (e.g., at least 3 of the genes listed in Tables 1-6b or at least three of the ISGs listed in Table 39), and determining at least one clinical parameter for the patient (e.g., age, tumor size, node status, tumor stage), and using the expression of said plurality of test genes and the clinical parameter(s), in classifying the cancer (e.g., determining the prognosis of the cancer in the patient, or predicting the cancer outcome, response to chemotherapy, the likelihood of cancer recurrence or probability of post-surgery distant metastasis-free survival). In some embodiments, the BCRGs, TCRGs, HLAGs, and/or OCPGs information and the clinical parameter information are combined to yield a quantitative (e.g., numerical) evaluation or score of the prognosis of the cancer in the patient, or cancer outcome, the likelihood of cancer recurrence or probability of post-surgery distant metastasis-free survival. In some embodiments, the expression level of the genes selected from the BCRGs, TCRGs, HLAGs, and OCPGs and the clinical parameter information are combined to yield a quantitative evaluation score of the prognosis of the cancer in the patient, or cancer outcome, the likelihood of cancer recurrence or probability of post-surgery distant metastasis-free survival. In some embodiments, the expression level of the genes selected from the BCRGs, TCRGs, HLAGs, and OCPGs and the clinical parameter information are combined with the expression level of the PGR, ABCC5 and/or ESR1 genes to yield a quantitative evaluation score of the prognosis of the cancer in the patient, or cancer outcome, the likelihood of cancer recurrence or probability of post-surgery distant metastasis-free survival.

In another aspect, the present disclosure provides a method for classifying cancer in a patient which comprises: determining in a sample from a patient the expression of at least 4, 8, or 12 test genes selected from BCRGs, TCRGs, HLAGs, and OCPGs (e.g., selected from Tables 1, 2, 3, 4 and/or 5), and at least 4, 8, or 12 test genes selected from CCGs (e.g., selected from Table 7), and determining at least one clinical parameter for the patient (e.g., age, tumor size, node status, tumor stage), and using the expression of the test genes in classifying the cancer (e.g., determining the prognosis of the cancer in the patient, predicting the cancer outcome, response to chemotherapy, the likelihood of cancer recurrence or probability of post-surgery distant metastasis-free survival). Thus, in one aspect the disclosure provides a method for classifying cancer comprising: determining in a sample from a patient the expression of a panel of genes comprising at least 4, 8, or 12 test genes selected from Tables 1, 2, 3, 4 and/or 5 and at least 4, 8, or 12 genes selected from Table 7, and determining at least one clinical parameter for the patient (e.g., age, tumor size, node status, tumor stage), and using the expression of the panel of genes in classifying the cancer. In some embodiments, the expression level of the genes selected from the BCRGs, TCRGs, HLAGs, OCPGs, and CCGs and the clinical parameter information are combined to yield a quantitative evaluation score of the prognosis of the cancer in the patient, or cancer outcome, the likelihood of cancer recurrence or probability of post-surgery distant metastasis-free survival.

In some embodiments, a treatment regimen comprising chemotherapy is recommended, prescribed or administered based at least in part on the expression levels of said BCRGs, TCRGs, HLAGs, or OCPGs, and said cell cycle genes. In some embodiments, a treatment regimen comprising surgical resection or radiation is recommended prescribed or administered in addition to based at least in part on the expression levels of said BCRGs, TCRGs, HLAGs, or OCPGs, and said cell cycle genes. In some embodiments, a treatment regimen comprising surgical resection or radiation is not recommended prescribed or administered based at least in part on the expression levels of said BCRGs, TCRGs, HLAGs, or OCPGs, and said cell cycle genes.

The present disclosure further provides a method for determining in a patient the prognosis of cancer or the likelihood of cancer recurrence, which comprises: determining the expression of a plurality of test genes comprising (1) at least 4, 6, 8, 10, 12 or 15 or more genes selected from the BCRGs, TCRGs, HLAGs, and OCPGs (e.g., in Table 1) and using the expression of said plurality of test genes in determining the prognosis of the cancer in the patient, or predicting the cancer outcome, the likelihood of cancer recurrence or probability of post-surgery distant metastasis-free survival. In some embodiments, the method comprises correlating an increased or higher expression level of the genes selected from BCRGs, TCRGs, HLAGs, and bpOCPGs, to a good or better prognosis, decreased likelihood of cancer recurrence, or increased probability of post-surgery distant metastasis-free survival. In some embodiments, the method comprises correlating no increase or lower expression levels of the genes selected from BCRGs, TCRGs, HLAGs, and bpOCPGs, to a bad or worse prognosis, increased likelihood of cancer recurrence, or decreased probability of post-surgery distant metastasis-free survival. In some embodiments, the method comprises correlating an increased or higher expression level of the wpOCPGs, to a bad or worse prognosis, bad or worse cancer outcome, increased likelihood of cancer recurrence, or decreased probability of post-surgery distant metastasis-free survival. In some embodiments, the method comprises correlating no increase, or lower expression level of the wpOCPGs, to a good or better prognosis, decreased likelihood of cancer recurrence, or increased probability of post-surgery distant metastasis-free survival. In some embodiments the prognosis includes likelihood of response to chemotherapy. In a specific aspect, the cancer is lung cancer, bladder cancer, prostate cancer, brain cancer, or breast cancer. In another specific aspect, the cancer is breast cancer. In yet another specific aspect, the cancer is ER positive breast cancer.

In another aspect, the present disclosure provides a method for determining the prognosis in a patient having breast cancer or the likelihood of breast cancer recurrence as described in the aspects and embodiments of the disclosure disclosed herein and further comprises: determining in a sample from the patient the expression of the PGR gene, and using the expression of the PGR gene in determining the prognosis of the breast cancer in the patient, or predicting the breast cancer outcome, or the likelihood of breast cancer recurrence or probability of post-surgery distant metastasis-free survival. In some embodiments, the method comprises correlating an increased expression level of the PGR gene, in patients who have received hormonal therapy, to a good or better prognosis, decreased likelihood of cancer recurrence, and increased probability of post-surgery distant metastasis-free survival. Conversely, the method comprises correlating an increased expression level of the PGR gene, in patients who have not received hormonal therapy, to a bad or worse prognosis, increased likelihood of cancer recurrence, and decreased probability of post-surgery distant metastasis-free survival. Furthermore, in some embodiments the method comprises correlating an increased expression level of the PGR gene to an increased likelihood of response to hormonal treatment. In some embodiments the method comprises correlating a decreased expression level of the PGR gene to a decreased likelihood of response to hormonal treatment.

The present disclosure further provides a method for determining in a patient the prognosis of cancer or the likelihood of cancer recurrence, which comprises: determining the expression of a plurality of test genes comprising (1) at least 4, 6, 8, 10, 12 or 15 or more cell-cycle genes (e.g., CCGs in Table 7, Panel F in Table 16 or Panel H in Table 17) and at least 4, 6, 8, 10, 12 or 15 or more genes selected from the BCRGs, TCRGs, HLAGs, and OCPGs (e.g., in Table 1) and using the expression of said plurality of test genes in determining the prognosis of the cancer in the patient, predicting the cancer outcome, or the likelihood of cancer recurrence or probability of post-surgery distant metastasis-free survival. In some embodiments, the method comprises correlating an overall increased expression level of cell-cycle genes, i.e., CCGs, to poor or worse prognosis of the cancer in the patient, poor or worse cancer outcome, increased likelihood of cancer recurrence, or decreased probability of post-surgery distant metastasis-free survival. In some embodiments, the method comprises correlating no increase or lower expression level of cell-cycle genes, i.e., CCGs, to good or better prognosis of the cancer in the patient, good or better cancer outcome, decreased likelihood of cancer recurrence, or increased probability of post-surgery distant metastasis-free survival. In some embodiments, the method comprises correlating an overall increased or higher expression level of BCRGs, TCRGs, HLAGs, and bpOCPGs to good or better prognosis, of the cancer in the patient, good, or better, cancer outcome, or decreased likelihood of cancer recurrence, or increased probability of post-surgery distant metastasis-free survival. In some embodiments, the method comprises correlating an increased or higher expression level of the genes selected from BCRGs, TCRGs, HLAGs, and bpOCPGs, to a good or better prognosis, decreased likelihood of cancer recurrence, or increased probability of post-surgery distant metastasis-free survival. In some embodiments, the method comprises correlating no increase or lower expression levels of the genes selected from BCRGs, TCRGs, HLAGs, and bpOCPGs, to a bad or worse prognosis, increased likelihood of cancer recurrence, or decreased probability of post-surgery distant metastasis-free survival. In some embodiments, the method comprises correlating an increased or higher expression level of the wpOCPGs, to a bad or worse prognosis, increased likelihood of cancer recurrence, or decreased probability of post-surgery distant metastasis-free survival. In some embodiments, the method comprises correlating no increase, or lower expression level of the wpOCPGs, to a good or better prognosis, decreased likelihood of cancer recurrence, or increased probability of post-surgery distant metastasis-free survival. In some embodiments the methods include correlating these expression levels with likelihood of response to chemotherapy. In a specific aspect, the cancer is lung cancer, bladder cancer, prostate cancer, brain cancer, or breast cancer. In another specific aspect, the cancer is breast cancer. In yet another specific aspect, the cancer is ER positive breast cancer.

The present disclosure further provides a method for determining in a patient the prognosis of cancer or the likelihood of cancer recurrence, which comprises: determining the expression of a plurality of test genes comprising (1) at least 4, 6, 8, 10, 12, or 15, or more cell-cycle genes (e.g., CCGs in Table 7, Panel F in Table 16, or Panel H in Table 17) and at least 4, 6, 8, 10, 12, or 15, or more genes selected from the BCRGs, TCRGs, HLAGs, and OCPGs (e.g., in Table 1) and/or (2) at least one of the ABCC5 gene and the PGR gene or both, together or separately in one or more samples from the patient, and using the expression of said plurality of test genes in determining the prognosis of the cancer in the patient, or predicting the cancer outcome, the likelihood of cancer recurrence or probability of post-surgery distant metastasis-free survival. In some embodiments, the method comprises correlating an overall increased expression level of cell-cycle genes, i.e., CCGs, to poor or worse prognosis of the cancer in the patient, poor or worse cancer outcome, increased likelihood of cancer recurrence, or decreased probability of post-surgery distant metastasis-free survival. In some embodiments, the method comprises correlating no increase or lower expression level of cell-cycle genes, i.e., CCGs, to good or better prognosis of the cancer in the patient, good or better cancer outcome, decreased likelihood of cancer recurrence, or increased probability of post-surgery distant metastasis-free survival. In some embodiments, the method comprises correlating an increased or higher expression level of the genes selected from BCRGs, TCRGs, HLAGs, and bpOCPGs, to a good or better prognosis, decreased likelihood of cancer recurrence, or increased probability of post-surgery distant metastasis-free survival. In some embodiments, the method comprises correlating no increase or lower expression levels of the genes selected from BCRGs, TCRGs, HLAGs, and bpOCPGs, to a bad or worse prognosis, increased likelihood of cancer recurrence, or decreased probability of post-surgery distant metastasis-free survival. In some embodiments, the method comprises correlating an increased or higher expression level of the wpOCPGs, to a bad or worse prognosis, increased likelihood of cancer recurrence, or decreased probability of post-surgery distant metastasis-free survival. In some embodiments, the method comprises correlating no increase, or lower expression level of the wpOCPGs, to a good or better prognosis, decreased likelihood of cancer recurrence, or increased probability of post-surgery distant metastasis-free survival. In some embodiments, the method comprises correlating an increased level of ABCC5 gene expression to poor or worse prognosis of the cancer in the patient, poor or worse cancer outcome, increased likelihood of cancer recurrence, or decreased probability of post-surgery distant metastasis-free survival. In contrast, in some embodiments, the method comprises correlating an increased level of PGR gene expression, in patients who have received hormonal therapy, to better prognosis of the cancer in the patient, better cancer outcome, decreased likelihood of cancer recurrence, or increased probability of post-surgery distant metastasis-free survival. Conversely, in some embodiments, the method comprises correlating an increased level of PGR gene expression, in patients who have not received hormonal therapy, to good or better prognosis of the cancer in the patient, better cancer outcome, decreased likelihood of cancer recurrence, or increased probability of post-surgery distant metastasis-free survival. In a specific aspect, the cancer is lung cancer, bladder cancer, prostate cancer, brain cancer, or breast cancer. In some embodiments the methods include correlating these expression levels with likelihood of response to chemotherapy. In another specific aspect, the cancer is breast cancer. In yet another specific aspect, the cancer is ER positive breast cancer.

The present disclosure further provides a method for determining in a patient the prognosis of breast cancer or the likelihood of cancer recurrence in a patient diagnosed with breast cancer, which comprises: determining the expression of a plurality of test genes comprising (1) at least 4, 6, 8, 10, 12 or 15 or more cell-cycle genes (e.g., CCGs in Table 7, Panel F in Table 16, or Panel H in Table 17) and at least 4, 6, 8, 10, 12 or 15 or more genes selected from the BCRGs, TCRGs, HLAGs, and OCPGs (e.g., in Table 1) and/or (2) at least one of the ABCC5 gene and the PGR gene or both, together or separately in one or more samples from the patient, and using the expression of said plurality of test genes in determining the prognosis of the breast cancer in the patient, or predicting the breast cancer outcome, the likelihood of cancer recurrence or probability of post-surgery distant metastasis-free survival. In some embodiments, the method comprises correlating an overall increased expression level of cell-cycle genes, i.e., CCGs, to poor or worse prognosis of the breast cancer in the patient, poor or worse breast cancer outcome, increased likelihood of cancer recurrence, or decreased probability of post-surgery distant metastasis-free survival. In some embodiments, the method comprises correlating no increase or lower expression level of cell-cycle genes, i.e., CCGs, to good or better prognosis of the breast cancer in the patient, good or better breast cancer outcome, decreased likelihood of cancer recurrence, or increased probability of post-surgery distant metastasis-free survival. In some embodiments, the method comprises correlating an increased or higher expression level of the genes selected from BCRGs, TCRGs, HLAGs, and bpOCPGs, to a good or better prognosis, decreased likelihood of cancer recurrence, or increased probability of post-surgery distant metastasis-free survival. In some embodiments, the method comprises correlating no increase or lower expression levels of the genes selected from BCRGs, TCRGs, HLAGs, and bpOCPGs, to a bad or worse prognosis, increased likelihood of cancer recurrence, or decreased probability of post-surgery distant metastasis-free survival. In some embodiments, the method comprises correlating an increased or higher expression level of the wpOCPGs, to a bad or worse prognosis, increased likelihood of cancer recurrence, or decreased probability of post-surgery distant metastasis-free survival. In some embodiments, the method comprises correlating no increase, or lower expression level of the wpOCPGs, to a good or better prognosis, decreased likelihood of cancer recurrence, or increased probability of post-surgery distant metastasis-free survival. In some embodiments, the method comprises correlating an increased level of ABCC5 gene expression to poor or worse prognosis of the breast cancer in the patient, poor or worse breast cancer outcome, increased likelihood of cancer recurrence, or decreased probability of post-surgery distant metastasis-free survival. In contrast, in some embodiments, the method comprises correlating an increased level of PGR gene expression, in patients who have received hormonal therapy, to better prognosis of the breast cancer in the patient, better breast cancer outcome, decreased likelihood of cancer recurrence, or increased probability of post-surgery distant metastasis-free survival. Conversely, in some embodiments, the method comprises correlating an increased level of PGR gene expression, in patients who have not received hormonal therapy, to good or better prognosis of the breast cancer in the patient, better breast cancer outcome, decreased likelihood of cancer recurrence, or increased probability of post-surgery distant metastasis-free survival. In some embodiments the methods include correlating these expression levels with likelihood of response to chemotherapy.

In some embodiments of the methods described above, the patient is ER+ and node negative. In some embodiments, the patient is ER+ and node negative, has undergone surgery to remove the tumor in her breast, and is placed on hormone therapy. In some embodiments of the methods described above, the patient is ER+ and node positive. In some embodiments of the methods described above, the ER status of the tumor is determined prior to determination of a gene expression profile or signature as described herein. In some embodiments of the methods described above, the ER status of the tumor is determined prior to determination of a gene expression profile or signature as described herein by IHC. In some embodiments of the methods described above, the ER status of the tumor is determined in conjunction with the determination of a gene expression profile or signature as described herein (e.g., the status of the ER is determined by gene expression analysis of the ESR1 gene, the status of the ER is determined by gene expression analysis with primers for amplifying an ESR1 gene product or a corresponding cDNA and a probe that corresponds to the amplification product). In some embodiments of the methods described above, the ER status of the tumor is determined in conjunction with determination of the gene expression profile or signature as described herein to confirm or not confirm another analysis of ER status in the tumor (e.g., by IHC).

As described herein, PR status and/or ER status is optionally evaluated by IHC prior to the evaluation of the gene expression profiles or signatures as described herein. Any number of methods can be used to detect ER or PR status by IHC as is known by the skilled artisan. Preferred IHC methods for determining ER and PR status include the ER/PR pharmDx assay kit (Dako, Glostrup, Denmark), the method of Harvey et al. ((1999) J Clin Oncol 17:1474-1481) for ER, or the method of Moshin et al. (2004) Mod Pathol 17:1545-1554.

The prognosis and treatment methods that involve determining a test value may further include a step of comparing the test value to one or more reference values, and correlating the test value to, e.g., a good or poor prognosis, an increased or decreased likelihood of recurrence, an increased or decreased likelihood of recurrence or metastasis-free survival, an increased or decreased likelihood of response to the particular treatment regimen (such as chemotherapy, and surgical resection), etc. In some embodiments, the expression data from BCRGs, TCRG, HLAGs, and OCPGs are combined into one test value, which may then be compared against a reference value for the combined score. In other embodiments, the BCRGs, TCRGs, HLAGs and OCPGs expression data are used to provide a discrete ISG/OCPG test value, which is then optionally combined with other parameters such as other gene expression signatures or clinical parameters. In some embodiments a test value greater than the reference value is correlated to an increased likelihood of response to treatment comprising chemotherapy. In some embodiments the test value is correlated to an increased likelihood of response to treatment (e.g., treatment comprising chemotherapy), poor prognosis, an increased likelihood of recurrence, and/or a decreased likelihood of recurrence or metastasis-free survival if the test value exceeds the reference value by at least some amount (e.g., at least 0.5, 0.75, 0.85, 0.90, 0.95, 1, 2, 3, 4, 5, 6, 7, 8, 9, or 10 or more fold or standard deviations).

The prognosis and treatment methods that involve determining a test value may further include a step of comparing the test value to one or more reference values, and correlating the test value to, e.g., a good or poor prognosis, an increased or decreased likelihood of recurrence, an increased or decreased likelihood of recurrence or metastasis-free survival, an increased or decreased likelihood of response to the particular treatment regimen, etc. In some embodiments, the expression data from BCRGs, TCRG, HLAGs, OCPGs, and CCPs are combined into one test value, which may then be compared against a reference value for the combined score. In other embodiments, the BCRGs, TCRGs, HLAGs, OCPGs and CCPs expression data are used to provide a discrete ISG/OCPG/CCP test value, which is then optionally combined with other parameters such as other gene expression signatures or clinical parameters. In some embodiments a test value greater than the reference value is correlated to an increased likelihood of response to treatment comprising chemotherapy. In some embodiments the test value is correlated to an increased likelihood of response to treatment (e.g., treatment comprising chemotherapy), poor prognosis, an increased likelihood of recurrence, and/or a decreased likelihood of recurrence or metastasis-free survival if the test value exceeds the reference value by at least some amount (e.g., at least 0.5, 0.75, 0.85, 0.90, 0.95, 1, 2, 3, 4, 5, 6, 7, 8, 9, or 10 or more fold or standard deviations).

In another aspect, the prognosis and treatment methods that involve determining a test value may further include a step of comparing the test value to one or more reference values, and correlating the test value to, e.g., a good/better or poor/worse prognosis, an increased or decreased likelihood of recurrence, an increased or decreased likelihood of recurrence or metastasis-free survival, an increased or decreased likelihood of response to the particular treatment regimen, etc. In some embodiments, the expression data from BCRGs, TCRG, HLAGs, and OCPGs, and are combined with ABCC5 and/or PGR expression data into one test value, which may then be compared against a reference value for the combined score. In other embodiments, the BCRGs, TCRGs, HLAGs and OCPGs expression data are used to provide a discrete ISG/OCPG test value, which is then combined with ABCC5 and/or PGR expression data. In some embodiments a test value greater than the reference value is correlated to an increased likelihood of response to treatment comprising chemotherapy. In some embodiments the test value is correlated to an increased likelihood of response to treatment (e.g., treatment comprising chemotherapy), poor prognosis, an increased likelihood of recurrence, and/or a decreased likelihood of recurrence or metastasis-free survival if the test value exceeds the reference value by at least some amount (e.g., at least 0.5, 0.75, 0.85, 0.90, 0.95, 1, 2, 3, 4, 5, 6, 7, 8, 9, or 10 or more fold or standard deviations).

In another aspect, the prognosis and treatment methods that involve determining a test value may further include a step of comparing the test value to one or more reference values, and correlating the test value to, e.g., a good/better or poor/worse prognosis, an increased or decreased likelihood of recurrence, an increased or decreased likelihood of recurrence or metastasis-free survival, an increased or decreased likelihood of response to the particular treatment regimen, etc. In some embodiments, the expression data from CCP, BCRGs, TCRG, HLAGs, and OCPGs, and are combined with ABCC5 and/or PGR expression data into one test value, which may then be compared against a reference value for the combined score. In other embodiments, the CCP, BCRGs, TCRGs, HLAGs and OCPGs expression data are used to provide a discrete ISG/OCPG/CCG test value, which is then combined with ABCC5 and/or PGR expression data. In some embodiments a test value greater than the reference value is correlated to an increased likelihood of response to treatment comprising chemotherapy. In some embodiments the test value is correlated to an increased likelihood of response to treatment (e.g., treatment comprising chemotherapy), poor prognosis, an increased likelihood of recurrence, and/or a decreased likelihood of recurrence or metastasis-free survival if the test value exceeds the reference value by at least some amount (e.g., at least 0.5, 0.75, 0.85, 0.90, 0.95, 1, 2, 3, 4, 5, 6, 7, 8, 9, or 10 or more fold or standard deviations).

In some embodiments, the method of determining the likelihood of response to a particular treatment regimen comprises (1) determining in a sample from a patient having cancer the expression of a panel of genes in said sample including at least 4 or at least 8 genes selected from BCRGs, TCRGs, HLAGs and OCPGs; (2) providing a test value by (a) weighting the determined expression of each of a plurality of test genes selected from the panel of genes with a predefined coefficient, and (b) combining the weighted expression to provide the test value, wherein the BCRGs, TCRGs, HLAGs and OCPGS are weighted to contribute at least 50%, at least 75% or at least 85% of the test value; and (3)(a) correlating a test value that is greater than some reference to an increased likelihood of response to the particular treatment regimen (e.g., a treatment regimen comprising chemotherapy, a treatment regimen comprising hormonal therapy), or (b) correlating a test value that is not greater than some reference to no increased likelihood of response to the particular treatment regimen (e.g., a treatment regimen comprising chemotherapy, a treatment regimen comprising hormonal therapy).

In some embodiments, the method of determining the likelihood of response to a particular treatment regimen comprises (1) determining in a sample from a patient having breast cancer the expression of a panel of genes in said sample including at least 4 or at least 8 genes selected from BCRGs, TCRGs, HLAGs and OCPGs; (2) providing a test value by (a) weighting the determined expression of each of a plurality of test genes selected from the panel of genes with a predefined coefficient, and (b) combining the weighted expression to provide the test value, wherein the BCRGs, TCRGs, HLAGs and OCPGS are weighted to contribute at least 50%, at least 75% or at least 85% of the test value; (3) (a) correlating a test value that is greater than some reference to an increased likelihood of response to the particular treatment regimen (e.g., a treatment regimen comprising chemotherapy, a treatment regimen comprising hormonal therapy), or (b) correlating a test value that is not greater than some reference to no increased likelihood of response to the particular treatment regimen (e.g., a treatment regimen comprising chemotherapy, a treatment regimen comprising hormonal therapy).

In some embodiments, the method of determining the likelihood of response to a particular treatment regimen comprises (1) determining in a sample from a patient having breast cancer the expression of a panel of genes in said sample including at least 4 or at least 8 genes selected from BCRGs, TCRGs, HLAGs and OCPGs; (2) providing a test value by (a) weighting the determined expression of each of a plurality of test genes selected from the panel of genes with a predefined coefficient, and (b) combining the weighted expression to provide the test value, wherein the BCRGs, TCRGs, HLAGs and OCPGS are weighted to contribute at least 50%, at least 75% or at least 85% of the test value; (3) determining in a sample from the patient the expression of ABCC5 and/or PGR; and (4)(a) correlating a test value that is greater than some reference and/or ABCC5 expression that is greater than some reference and/or PGR expression that is greater than some reference to an increased likelihood of response to the particular treatment regimen (e.g., a treatment regimen comprising chemotherapy, a treatment regimen comprising hormonal therapy), or (b) correlating a test value that is not greater than some reference and/or ABCC5 expression that is not greater than some reference and/or PGR expression that is not greater than some reference to no increased likelihood of response to the particular treatment regimen (e.g., a treatment regimen comprising chemotherapy, a treatment regimen comprising hormonal therapy).

In some embodiments, the method of determining the likelihood of response to a particular treatment regimen comprises (1) determining in a sample from a patient having breast cancer the expression of a panel of genes in said sample including at least 4 or at least 8 cell-cycle genes and at least 4 or at least 8 genes selected from BCRGs, TCRGs, HLAGs and OCPGs; (2) providing a test value by (a) weighting the determined expression of each of a plurality of test genes selected from the panel of genes with a predefined coefficient, and (b) combining the weighted expression to provide the test value, wherein the cell-cycle genes, BCRGs, TCRGs, HLAGs and OCPGS are weighted to contribute at least 50%, at least 75% or at least 85% of the test value; (3) (a) correlating a test value that is greater than some reference to an increased likelihood of response to the particular treatment regimen (e.g., a treatment regimen comprising chemotherapy, a treatment regimen comprising hormonal therapy), or (b) correlating a test value that is not greater than some reference to no increased likelihood of response to the particular treatment regimen (e.g., a treatment regimen comprising chemotherapy, a treatment regimen comprising hormonal therapy).

In some embodiments, the method of determining the likelihood of response to a particular treatment regimen comprises (1) determining in a sample from a patient having breast cancer the expression of a panel of genes in said sample including at least 4 or at least 8 cell-cycle genes and at least 4 or at least 8 genes selected from BCRGs, TCRGs, HLAGs and OCPGs; (2) providing a test value by (a) weighting the determined expression of each of a plurality of test genes selected from the panel of genes with a predefined coefficient, and (b) combining the weighted expression to provide the test value, wherein the cell-cycle genes, BCRGs, TCRGs, HLAGs and OCPGS are weighted to contribute at least 50%, at least 75% or at least 85% of the test value; (3) determining in a sample from the patient the expression of ABCC5 and/or PGR; and (4)(a) correlating a test value that is greater than some reference and/or ABCC5 expression that is greater than some reference and/or PGR expression that is greater than some reference to an increased likelihood of response to the particular treatment regimen (e.g., a treatment regimen comprising chemotherapy, a treatment regimen comprising hormonal therapy), or (b) correlating a test value that is not greater than some reference and/or ABCC5 expression that is not greater than some reference and/or PGR expression that is not greater than some reference to no increased likelihood of response to the particular treatment regimen (e.g., a treatment regimen comprising chemotherapy, a treatment regimen comprising hormonal therapy).

In some embodiments, the panel of genes in addition to the genes selected from the BCRGs, TCRGs, HLAGs, and OCPGs, include at least 2, 4, 5, 6, 7, 8, 9, 10, 12, 14, 16, 18, 20, 25, 30, 35, 40, 45, 50, 60, 70, 80, 90, 100 or more cell-cycle genes. In some embodiments the test genes are weighted such that the cell-cycle genes are weighted to contribute at least 50%, at least 55%, at least 60%, at least 65%, at least 75%, at least 80%, at least 85%, at least 90%, at least 95%, at least 99% or 100% of the test value. In some embodiments 20%, 25%, 30%, 35%, 40%, 45%, 50%, 55%, 60%, 65%, 75%, 80%, 85%, 90%, 95%, or at least 99% or 100% of the plurality of test genes are cell-cycle genes.

In some embodiments, the panel of genes includes at least 2, 4, 5, 6, 7, 8, 9, or 10 or more BCRGs. In some embodiments the test genes are weighted such that the BCRGs are weighted to contribute at least 1%, at least 2%, at least 3%, at least 4%, at least 5%, at least 10%, at least 15%, at least 20%, at least 25%, at least 30% or at least 40% of the test value. In some embodiments 1%, 2%, 3%, 4%, 5%, 6%, 7%, 8%, 9%, 10%, 15%, 20%, 30%, 40%, 50%, or at least 55%, or at least 60%, or at least 65%, or at least 70% or at least 75%, or at least 80%, or at least 85%, or at least 90% of the plurality of test genes are BCRGs.

In some embodiments, the panel of genes includes at least 2, 4, 5, 6, 7, 8, 9, or 10 or more TCRGs. In some embodiments the test genes are weighted such that the TCRGs are weighted to contribute at least 1%, at least 2%, at least 3%, at least 4%, at least 5%, at least 10%, at least 15%, at least 20%, at least 25%, at least 30% or at least 40% of the test value. In some embodiments 1%, 2%, 3%, 4%, 5%, 6%, 7%, 8%, 9%, 10%, 15%, 20%, 30%, 40%, 50%, or at least 55%, or at least 60%, or at least 65%, or at least 70% or at least 75%, or at least 80%, or at least 85%, or at least 90% of the plurality of test genes are TCRGs.

In some embodiments, the panel of genes includes at least 2, 4, 5, 6, 7, 8, 9, or 10 or more HLAGs. In some embodiments the test genes are weighted such that the HLAGs are weighted to contribute at least 1%, at least 2%, at least 3%, at least 4%, at least 5%, at least 10%, at least 15%, at least 20%, at least 25%, at least 30% or at least 40% of the test value. In some embodiments 1%, 2%, 3%, 4%, 5%, 6%, 7%, 8%, 9%, 10%, 15%, 20%, 30%, 40%, 50%, or at least 55%, or at least 60%, or at least 65%, or at least 70% or at least 75%, or at least 80%, or at least 85%, or at least 90% of the plurality of test genes are HLAGs.

In some embodiments, the panel of genes includes at least 2, 4, 5, 6, 7, 8, 9, or 10 or more OCPGs. In some embodiments the test genes are weighted such that the OCPGs are weighted to contribute at least 1%, at least 2%, at least 3%, at least 4%, at least 5%, at least 10%, at least 15%, at least 20%, at least 25%, at least 30% or at least 40% of the test value. In some embodiments 1%, 2%, 3%, 4%, 5%, 6%, 7%, 8%, 9%, 10%, 15%, 20%, 30%, 40%, 50%, or at least 55%, or at least 60%, or at least 65%, or at least 70% or at least 75%, or at least 80%, or at least 85%, or at least 90% of the plurality of test genes are OCPGs.

In some embodiments, the plurality of test genes includes at least 2, 3 or 4 ISGs and/or OCPGs, which constitute at least 50%, 75% or 80% of the plurality of test genes, and preferably 100% of the plurality of test genes. In some embodiments, the plurality of test genes includes at least 5, 6 or 7, or at least 8 ISGs and or OCPGs, which constitute at least 20%, 25%, 30%, 40%, 50%, 60%, 70%, 75%, 80% or 90% of the plurality of test genes, and preferably 100% of the plurality of test genes. Thus in some embodiments the plurality of test genes comprises at least some number of ISGs and or OCPGS (e.g., at least 3, 4, 5, 6, 7, 8, 9, 10, 15, 20, 25, 30, 35, 40, 45, 50 or more ISGs) and this plurality of ISGs comprises the top 2, 3, 4, 5, 6, 7, 8, 9, 10, 11, 12, 13, 14, 15, 20, 25, 30, 35, 40 or more ISGs and or OCPGs listed in any of Tables 1, 6A, 6B, 8, 9, 30, 31, 32, or 33. In some embodiments the plurality of test genes comprises at least some number of ISGs and or OCPGs (e.g., at least 3, 4, 5, 6, 7, 8, 9, 10, 15, 20, 25, 30, 35, 40, 45, 50 or more ISGs and or OCPGs) and this plurality of ISGs and or OCPGS comprises at least 1, 2, 3, 4, 5, 6, 7, 8, 9, 10, 15, or 20 of the following genes: CEP57, LITAF, ZFP36L2, SLC35E3, SLC4A8, HLA-DRB1/3, GPRC5A, HLA-DPA1, IGL1, CALD1, HLA-DPB1, ERP29, RACGAP1, IGLL3P, TCRA/D, IGHM, HLA-DRA, CD74, HLA-DMA and PDGFB. In some embodiments the plurality of test genes comprises at least some number of ISGs and/or OCPGs (e.g., at least 3, 4, 5, 6, 7, 8, 9, 10, 15, 20, 25, 30, 35, 40, 45, 50 or more ISGs and or OCPGs) and this plurality of ISGs comprises any one, two, three, four, five, six, seven, eight, nine, or ten or all of gene numbers 1 & 2, 1 to 3, 1 to 4, 1 to 5, 1 to 6, 1 to 7, 1 to 8, 1 to 9, or 1 to 10 of any of Tables 1, 6A, 6B, 8, 9, 30, 31, 32, or 33. In some embodiments the plurality of test genes comprises at least some number of ISGs and or OCPGs (e.g., at least 3, 4, 5, 6, 7, 8, 9, 10, 15, 20, 25, 30, 35, 40, 45, 50 or more ISGs and or OCPGs) and this plurality of ISGs and or OCPGs comprises any one, two, three, four, five, six, seven, eight, or nine or all of gene numbers 2 & 3, 2 to 4, 2 to 5, 2 to 6, 2 to 7, 2 to 8, 2 to 9, or 2 to 10 of any of Tables 1, 6A, 6B, 8, 9, 30, 31, 32, or 33. In some embodiments the plurality of test genes comprises at least some number of ISGs and/or OCPGs (e.g., at least 3, 4, 5, 6, 7, 8, 9, 10, 15, 20, 25, 30, 35, 40, 45, 50 or more ISGs and/or OCPGs) and this plurality of ISG and or OCPGs comprises any one, two, three, four, five, six, seven, or eight or all of gene numbers 3 & 4, 3 to 5, 3 to 6, 3 to 7, 3 to 8, 3 to 9, or 3 to 10 of any of Tables 1, 6A, 6B, 8, 9, 30, 31, 32, or 33. In some embodiments the plurality of test genes comprises at least some number of ISGs and/or OCPGs (e.g., at least 3, 4, 5, 6, 7, 8, 9, 10, 15, 20, 25, 30, 35, 40, 45, 50 or more ISGs and/or OCPGs) and this plurality of ISGs and/or OCPGs comprises any one, two, three, four, five, six, or seven or all of gene numbers 4 & 5, 4 to 6, 4 to 7, 4 to 8, 4 to 9, or 4 to 10 of any of Tables 1, 6A, 6B, 8, 9, 30, 31, 32, or 33. In some embodiments the plurality of test genes comprises at least some number of ISGs and/or OCPGs (e.g., at least 3, 4, 5, 6, 7, 8, 9, 10, 15, 20, 25, 30, 35, 40, 45, 50 or more ISGs and/or OCPGs) and this plurality of ISGs and/or OCPGs comprises any one, two, three, four, five, six, seven, eight, nine, 10, 11, 12, 13, 14, or 15 or all of gene numbers 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Tables 1, 6A, 6B, 8, 9, 30, 31, 32, or 33.

In some other embodiments, the plurality of test genes includes at least 8, 10, 12, 15, 20, 25 or 30 of ISGs and/or OCPGs, which constitute at least 20%, 25%, 30%, 40%, 50%, 60%, 70%, 75%, 80% or 90% of the plurality of test genes, and preferably 100% of the plurality of test genes. Panels of genes selected from BCRGs, TCRGs, HLAGs and OCPGs, alone or in combination with CCGs (e.g., 2, 3, 4, 5, or 6 CCGs) can accurately predict cancer prognosis, and in particular breast cancer prognosis. But addition of the ABCC5 and PGR genes significantly increases the prediction power. In some embodiments the panel comprises at least 2, 3, 4, 5, 6, 7, 8, 9, 10, 15, 20, 25, 30, 35, 40, 45, 50, 70, 80, 90, 100, 200, or more genes selected from BCRGs, TCRGs, HLAGs, OCPGs. In some embodiments the panel comprises the ABCC5 or PGR genes and at least 3, 4, 5, 6, 7, 8, 9, 10, 15, 20, 25, 30, 35, 40, 45, 50, 70, 80, 90, 100, 200, or more genes selected from BCRGs, TCRGs, HLAGs, and OCPGs. In some embodiments the panel comprises the ABCC5 and PGR genes and at least 2, 3, 4, 5, 6, 7, 8, 9, 10, 15, 20, 25, 30, 35, 40, 45, 50, 70, 80, 90, 100, 200, or more genes selected from BCRGs, TCRGs, HLAGs, and OCPGs. In some embodiments the panel comprises at least 10, 15, 20, or more genes selected from BCRGs, TCRGs, HLAGs, and OCPGs. In some embodiments the panel comprises between 5 and 100 genes selected from BCRGs, TCRGs, HLAGs, and OCPGs, between 7 and 40 genes selected from BCRGs, TCRGs, HLAGs, and OCPGs, between 5 and 25 genes selected from BCRGs, TCRGs, HLAGs, and OCPGs, between 10 and 20 genes selected from BCRGs, TCRGs, HLAGs, and OCPGs, or between 10 and 15 genes selected from BCRGs, TCRGs, HLAGs, and OCPGs. In some embodiments the genes selected from BCRGs, TCRGs, HLAGs, and OCPGs comprise at least a certain proportion of the panel. Thus, in some embodiments the panel comprises at least 25%, 30%, 40%, 50%, 60%, 70%, 75%, 80%, 85%, 90%, 95%, 96%, 97%, 98%, or 99% genes selected from BCRGs, TCRGs, HLAGs, and OCPGs. In some preferred embodiments the panel comprises at least 10, 15, 20, 25, 30, 35, 40, 45, 50, 70, 80, 90, 100, 200, or more genes selected from BCRGs, TCRGs, HLAGs, and OCPGs, and such genes selected from BCRGs, TCRGs, HLAGs, and OCPGs constitute of at least 50%, 60%, 70%, preferably at least 75%, 80%, 85%, more preferably at least 90%, 95%, 96%, 97%, 98%, or 99% or more of the total number of genes in the panel. In some embodiments the panel of genes selected from BCRGs, TCRGs, HLAGs, and OCPGs, comprises the genes in Table 1, 2, 3, 5, 6a or 6b. In some embodiments the panel comprises at least 2, 3, 4, 5, 6, 7, 8, 9, 10, 11, 12, 13, 14, 15, 20, 25, 30, or more of the genes in Table 1, 2, 3, 5, 6a or 6b. In some embodiments the disclosure provides a method of determining the prognosis in a breast cancer patient comprising determining the status of the genes selected from BCRGs, TCRGs, HLAGs, and OCPGs in any one of Table 1, 2, 3, 5, 6a or 6b and using the combined expression to determine the prognosis of the breast cancer. In some embodiments the disclosure provides a method of determining the prognosis in a breast cancer patient comprising determining the status of the genes selected from BCRGs, TCRGs, HLAGs, and OCPGs in any one of Table 1, 2, 3, 5, 6a or 6b, determining the status of the ABCC5 gene or the PGR gene or both, and using the combined expression to determine the prognosis of the breast cancer.

As used herein, “determining the status” of a gene (or panel of genes) refers to determining the presence, absence, or extent/level of some physical, chemical, or genetic characteristic of the gene or its expression product(s). Such characteristics include, but are not limited to, expression levels, activity levels, mutations, copy number, methylation status, etc.

In the context of BCRGs, TCRGs, HLAGs, OCPGs and CCGs as used to determine likelihood of response to a particular treatment regimen (e.g., a treatment regimen comprising chemotherapy), particularly useful characteristics include expression levels (e.g., mRNA, cDNA or protein levels) and activity levels. Characteristics may be assayed directly (e.g., by assaying a gene's expression level) or determined indirectly (e.g., assaying the level of a gene or genes whose expression level is correlated to the expression level of the gene).

“Abnormal status” means a marker's status in a particular sample differs from the status generally found in average samples (e.g., healthy samples, average diseased samples). Examples include mutated, elevated, decreased, present, absent, etc. An “elevated status” means that one or more of the above characteristics (e.g., expression or mRNA level) is higher than normal levels. Generally this means an increase in the characteristic (e.g., expression or mRNA level) as compared to an index value as discussed below. Conversely a “low status” means that one or more of the above characteristics (e.g., gene expression or mRNA level) is lower than normal levels. Generally this means a decrease in the characteristic (e.g., expression) as compared to an index value as discussed below. In this context, a “negative status” generally means the characteristic is absent or undetectable or, in the case of sequence analysis, there is a deleterious sequence variant (including full or partial gene deletion).

Gene expression can be determined either at the RNA level (i.e., mRNA or noncoding RNA (ncRNA)) (e.g., miRNA, tRNA, rRNA, snoRNA, siRNA and piRNA) or at the protein level. Measuring gene expression at the mRNA level includes measuring levels of cDNA corresponding to mRNA and can be determined by any known technique in the art, which include but are not limited to, qPCR, mircroarray, highthroughput RNA sequencing, etc. Levels of proteins in a sample can be determined by any known technique in the art, e.g., HPLC, mass spectrometry, or using antibodies specific to selected proteins (e.g., IHC, ELISA, etc.).

In some embodiments, the amount of RNA transcribed from the panel of genes including test genes is measured in the sample. In addition, the amount of RNA of one or more housekeeping genes in the sample is also measured, and used to normalize or calibrate the expression of the test genes. The terms “normalizing genes” and “housekeeping genes” are defined herein below.

In any embodiment of the disclosure involving a “plurality of test genes,” the plurality of test genes may include at least 2, 3 or 4 genes selected from BCRGs, TCRGs, HLAGs and OCRGs, which constitute at least 50%, 75% or 80% of the plurality of test genes, and preferably 100% of the plurality of test genes. In other such embodiments, the plurality of test genes includes at least 5, 6, 7, or at least 8 genes chosen from BCRGs, TCRGs, HLAGs, and OCPGs, which together constitute at least 20%, 25%, 30%, 40%, 50%, 60%, 70%, 75%, 80% or 90% of the plurality of test genes, and preferably 100% of the plurality of test genes. As will be clear from the context of this document, a panel of genes is a plurality of genes. In some embodiments these genes are assayed together in one or more samples from a patient.

In some embodiments, the plurality of test genes includes at least 8, 10, 12, 15, 20, 25 or 30 genes selected from BCRGs, TCRGs, HLAGs, and OCPGs which together constitute at least 20%, 25%, 30%, 40%, 50%, 60%, 70%, 75%, 80% or 90% of the plurality of test genes, and preferably 100% of the plurality of test genes.

In any embodiment of the disclosure involving a “plurality of test genes,” the plurality of test genes may include at least 2, 3 or 4 genes cell-cycle genes and at least 2, 3 or 4 genes selected from BCRGs, TCRGs, HLAGs and OCRGs, together which constitute at least 50%, 75% or 80% of the plurality of test genes, and preferably 100% of the plurality of test genes. In other such embodiments, the plurality of test genes includes at least 5, 6, 7, or at least 8 cell-cycle genes and at least 5, 6, 7, or at least 8 genes chosen from BCRGs, TCRGs, HLAGs, and OCPGs, which together constitute at least 20%, 25%, 30%, 40%, 50%, 60%, 70%, 75%, 80% or 90% of the plurality of test genes, and preferably 100% of the plurality of test genes. As will be clear from the context of this document, a panel of genes is a plurality of genes. In some embodiments these genes are assayed together in one or more samples from a patient.

In some embodiments, the plurality of test genes includes at least 8, 10, 12, 15, 20, 25 or 30 cell-cycle genes and at least 8, 10, 12, 15, 20, 25 or 30 genes selected from BCRGs, TCRGs, HLAGs, and OCPGs which together constitute at least 20%, 25%, 30%, 40%, 50%, 60%, 70%, 75%, 80% or 90% of the plurality of test genes, and preferably 100% of the plurality of test genes.

As will be apparent to a skilled artisan apprised of the present disclosure and the disclosure herein, “tumor sample” means any biological sample containing one or more tumor cells, or tumor-derived DNA, RNA or protein, and obtained from a an individual currently or previously diagnosed with cancer, an individual undergoing cancer treatment, or an individual not diagnosed with cancer but who presents with symptoms consistent with a cancer diagnosis. For example, a tissue sample obtained from a tumor tissue of an individual is a useful tumor sample in the present disclosure. The tissue sample can be an FFPE sample, or fresh frozen sample, and preferably contain largely tumor cells. A single malignant cell from a patient's tumor is also a useful tumor sample. Such a malignant cell can be obtained directly from the patient's tumor, or purified from the patient's bodily fluid (e.g., blood, urine). Thus, a bodily fluid such as blood, urine, sputum and saliva containing one or tumor cells, or tumor-derived DNA, RNA or proteins, can also be useful as a tumor sample for purposes of practicing the present disclosure.

Those skilled in the art are familiar with various techniques for determining the expression of a gene in a tissue or cell sample, which can be measured as the level of the mRNA transcribed from, or the protein encoded by, the gene. Useful techniques include, but are not limited to, microarray analysis (e.g., for assaying mRNA or microRNA expression, copy number, etc.), quantitative real-time PCR™ (“qRT-PCR™”, e.g., TaqMan™), immunoanalysis (e.g., ELISA, immunohistochemistry) The activity level of a polypeptide encoded by a gene may be used in much the same way as the expression level of the gene or polypeptide. Often higher activity levels indicate higher expression levels and while lower activity levels indicate lower expression levels. Thus, in some embodiments, the disclosure provides any of the methods discussed above, wherein the activity level of a polypeptide encoded by the CCG, BCRG, TCRG, HLAG or OCPG is determined rather than or in addition to the expression level of the gene. Those skilled in the art are familiar with techniques for measuring the activity of various such proteins, including those encoded by the CCG, BCRG, TCRG, HLAG and OCPG genes listed in herein, as listed in Tables 1 and 7, as and PGR, ESR1, and ERBB2. The methods of the disclosure may be practiced independent of the particular technique used.

In preferred embodiments, the expression of one or more normalizing (often called “housekeeping”) genes is also obtained for use in normalizing the expression of test genes. As used herein, “normalizing genes” referred to the genes whose expression is used to calibrate or normalize the measured expression of the gene of interest (e.g., test genes). Importantly, the expression of normalizing genes should be independent of cancer outcome/prognosis, and the expression of the normalizing genes is very similar among all the samples. The normalization ensures accurate comparison of expression of a test gene between different samples. For this purpose, housekeeping genes known in the art can be used. Housekeeping genes are well known in the art, with examples including, but are not limited to, GUSB (glucuronidase, beta), HMBS (hydroxymethylbilane synthase), SDHA (succinate dehydrogenase complex, subunit A, flavoprotein), UBC (ubiquitin C) and YWHAZ (tyrosine 3-monooxygenase/tryptophan 5-monooxygenase activation protein, zeta polypeptide). One or more housekeeping genes can be used. Preferably, at least 2, 3, 4, 5, 7, 10 or 15 housekeeping genes are used to provide a combined normalizing gene set. In one aspect, the normalizing genes are selected from those in Table A. In one aspect, the normalizing genes are selected from those in Table B. In one aspect, the set of normalizing genes are or are selected from CLTC, GUSB, HMBS, MMADHC, MRFAP1, PPP2CA, PSMA1, PSMC1, RPL13A, RPL37, RPL38, RPL4, RPL8, RPS29, SDHA, SLC25A3, TXNL1, UBA52, UBC and YWHAZ. In one aspect, the set of normalizing genes are or are selected from CLTC, MMADHC, MRFAP1, PPP2CA, PSMA1, PSMC1, RPL13A, RPL37, RPL38, RPL4, RPL8, RPS29, SLC25A3, TXNL1, and UBA52. The amount of gene expression of such normalizing genes can be averaged, combined together by straight additions or by a defined algorithm. Some examples of particularly useful housekeeper genes for use in the methods and compositions of the disclosure include those listed in Table A below. In particular, the disclosure is some aspects, relates to primers (e.g., primer pairs) or sets of primers for amplifying mRNA, or corresponding cDNA, that correspond to one or more and preferably two or more of these genes (e.g., as in sets of primer pairs for different genes). In particular, the disclosure is some aspects relates to probes or sets of probes (e.g., hybridization probes) for specifically detecting and/or quantitating the level of mRNA, or corresponding cDNA, that correspond to one or more and preferably two or more of these genes (e.g., as in sets of probes for different genes).

TABLE A

| | | Applied | |
|---|---|---|---|
| Gene | Entrez | Biosystems | RefSeq Accession |
| Symbol | GeneID | Assay ID | Nos. |
| CLTC* | 1213 | Hs00191535_m1 | NM_004859.3 |
| GUSB | 2990 | Hs99999908_m1 | NM_000181.2 |
| HMBS | 3145 | Hs00609297_m1 | NM_000190.3 |
| MMADHC* | 27249 | Hs00739517_g1 | NM_015702.2 |
| MRFAP1 * | 93621 | Hs00738144_g1 | NM_033296.1 |
| PPP2CA* | 5515 | Hs00427259_m1 | NM_002715.2 |
| PSMA1 * | 5682 | Hs00267631_m1 | |
| PSMC1* | 5700 | Hs02386942_g1 | NM_002802.2 |
| RPL13A* | 23521 | Hs03043885_g1 | NM_012423.2 |
| RPL37* | 6167 | Hs02340038_g1 | NM_000997.4 |
| RPL38* | 6169 | Hs00605263_g1 | NM_000999.3 |
| RPL4* | 6124 | Hs03044647_g1 | NM_000968.2 |
| RPL8* | 6132 | Hs00361285_g1 | NM_033301.1; |
| | | | NM_000973.3 |
| RPS29* | 6235 | Hs03004310_g1 | NM_001030001.1; |
| | | | NM_001032.3 |
| SDHA | 6389 | Hs00188166_m1 | NM_004168.2 |
| SLC25A3* | 6515 | Hs00358082_m1 | NM_213611.1; |
| | | | NM_002635.2; |
| | | | NM_005888.2 |
| TXNL1* | 9352 | Hs00355488_m1 | NR_024546.1; |
| | | | NM_004786.2 |
| UBA52* | 7311 | Hs03004332_g1 | NM_001033930.1; |
| | | | NM_003333.3 |
| UBC | 7316 | Hs00824723_m1 | NM_021009.4 |
| YWHAZ | 7534 | Hs00237047_m1 | NM_003406.3 |

*Subset of preferred housekeeping genes used in normalizing CCGs and generating CCP scores or other scores like ISG/OCPG scores or ISG/OCPG/CCG scores.

In the case of measuring RNA levels for the genes, one convenient and sensitive approach is real-time quantitative PCR™ (qPCR) assay, following a reverse transcription reaction. Typically, a cycle threshold (Cₜ) is determined for each test gene and each normalizing gene, i.e., the number of cycles at which the fluorescence from a qPCR reaction above background is detectable

The overall expression of the one or more normalizing genes can be represented by a “normalizing value” which can be generated by combining the expression of all normalizing genes, either weighted equally (straight addition or averaging) or by different predefined coefficients. For example, in a simplest manner, the normalizing value C_{tH} can be the cycle threshold (Cₜ) of one single normalizing gene, or an average of the Cₜ values of 2 or more, preferably 10 or more, or 15 or more normalizing genes, in which case, the predefined coefficient is 1/N, where N is the total number of normalizing genes used. Thus, C_{tH}=(C_{tH1}+C_{tH2}+ . . . C_{tHn})/N. As will be apparent to skilled artisans, depending on the normalizing genes used, and the weight desired to be given to each normalizing gene, any coefficients (from 0/N to N/N) can be given to the normalizing genes in weighting the expression of such normalizing genes. That is, C_{tH}=xC_{tH1}+yC_{tH2}+ . . . zC_{tHn}, wherein x+y+z=1.

As discussed above, the methods of the disclosure generally involve determining the level of expression of a panel of genes selected from BCRGs, TCRGs, HLAGs, and OCPGs, which can optionally be combined with CCGs and/or the PGR gene. With modern highthroughput techniques, it is often possible to determine the expression level of tens, hundreds or thousands of genes. Indeed, it is possible to determine the level of expression of the entire transcriptome (i.e., each transcribed sequence in the genome). Once such a global assay has been performed, one may then informatically analyze one or more subsets of transcripts (i.e., panels or, as often used herein, pluralities of test genes). After measuring the expression of hundreds or thousands of transcripts in a sample, for example, one may analyze (e.g., informatically) the expression of a panel or plurality of test genes comprising primarily genes selected from BCRGs, TCRGs, HLAGs, OCPGs and optionally CCGs and/or the PGR gene according to the present disclosure by combining the expression level values of the individual test genes to obtain a test value.

As will be apparent to a skilled artisan, the different prognostic value provided in the present disclosure represents the overall expression level of the plurality of test genes composed substantially of (or weighted to be represented substantially by) genes selected from BCRGs, TCRGs, HLAGs, and OCPGs, and optionally, CCGs and/or the PGR. In one embodiment, to provide a specific prognostic value in the methods of the disclosure, the normalized expression for a test gene can be obtained by normalizing the measured Cₜ for the test gene against the C_{tH}, i.e., ΔCₜ₁=(Cₜ₁−C_{tH}). Thus, the specific prognostic value representing the overall expression of the plurality of test genes can be provided by combining the normalized expression of all test genes, either by straight addition or averaging (i.e., weighted equally) or by a different predefined coefficient. For example, the simplest approach is averaging the normalized expression of all test genes: prognostic value=(ΔCₜ₁+ΔCₜ₂+ . . . +ΔCₜₙ)/n. As will be apparent to skilled artisans, depending on the test genes used, different weight can also be given to different test genes in the present disclosure. In each case where this document discloses using the expression of a plurality of genes (e.g., “determining [in a sample from the patient] the expression of a plurality of test genes” or “correlating increased expression of said plurality of test genes to an increased likelihood of response”), this includes in some embodiments using a test value representing or corresponding to the overall expression of this plurality of genes (e.g., “determining [in a sample from the patient] a test value representing the expression of a plurality of test genes” or “correlating an increased test value [or a test value above some reference value] representing the expression of said plurality of test genes to an increased likelihood of response”).

For example, the normalized expression for the ABCC5 gene can be obtained by normalizing the measured Cₜ for the ABCC5 gene against the C_{tH}, i.e., ΔC_{t(ABCC5)}=(C_{t(ABCC5)}−C_{tH}). Likewise, the normalized expression for the PGR gene can be obtained by normalizing the measured Cₜ for the PGR gene against the C_{tH}, i.e., ΔC_{t(PGR)}=(C_{t(PGR)}−C_{tH}). Again, for example, the normalized expression for the ABCC5 gene and/or PGR gene can be combined with a BCRG, TCRG, OCPG, and/or CCP value described above to provide a test value. Same or different weights can be assigned to different components using predefined coefficients.

It has been determined that, once the phenomenon reported herein for the genes chosen from the BCRGs, TCRGs, HLAGs, and OCPGs is appreciated and optionally CCGs and/or the PGR gene, the choice of individual genes for a test panel can, in some embodiments, be somewhat arbitrary. In other words, many CCGs, BCRGs, TCRGs, HLAGs, or OCPGs have been found to be very good surrogates for each other. Thus, any CCGs, BCRGs, TCRGs, HLAGs, or OCPGs (or panel of CCGs, BCRGs, TCRGs, HLAGs, or OCPGs) can be used in the various embodiments of the disclosure. In other embodiments of the disclosure, optimized CCGs, BCRGs, TCRGs, HLAGs, or OCPGs are used. One way of assessing whether particular genes will serve well in the methods and compositions of the disclosure is by assessing their correlation with the mean expression of CCGs, BCRGs, TCRGs, HLAGs, or OCPGs (e.g., all known CCGs, BCRGs, TCRGs, HLAGs, or OCPGs, a specific set of CCGs, BCRGs, TCRGs, HLAGs, or OCPGs, etc.). Those CCGs, BCRGs, TCRGs, HLAGs, or OCPGs that correlate particularly well with the mean are expected to perform well in assays of the disclosure, e.g., because these will reduce noise in the assay.

Some CCGs, BCRGs, TCRGs, HLAGs, or OCRGs do not correlate well with the mean (e.g., ABCC5's correlation to the mean is 0.097) for the CCG profile or a BCRG, TCRG, HLAG, or OCPG profile. In some embodiments of the present disclosure, such genes may be grouped, tested, analyzed, etc. separately from those that correlate well. This is especially useful if these non-correlated genes are independently associated with the clinical feature of interest (e.g., prognosis, therapy response, etc.). Again, ABCC5, an OCPG, is a good example, as it does not correlate with the CCG mean at all but it correlates well with prognosis. As shown in the example below, where ABCC5 remains a significant predictor of prognosis even in multivariate analysis with correlated CCP genes, ABCC5 adds prognostic information beyond CCGs that correlate well with the mean (e.g., Panel F). Thus, in some preferred embodiments of the disclosure, non-correlated genes are analyzed together with correlated genes. In some embodiments, a BCRG, TCRG, HLAG, or OCPG is non-correlated if its correlation to its respective mean (e.g., cluster mean as described in the Examples) is less than 0.5, 0.4, 0.3, 0.2, 0.10, 0.09, 0.08, 0.07, 0.06, 0.05, 0.04, 0.03, 0.02, 0.01 or less. In some embodiments, a CCG is non-correlated if its correlation to the CCG mean is less than 0.5, 0.4, 0.3, 0.2, 0.10, 0.09, 0.08, 0.07, 0.06, 0.05, 0.04, 0.03, 0.02, 0.01 or less.

The expression of individual BCRGs, TCRGs, HLAGs and OCPGs was compared to their respective cluster mean as described in the examples below in order to determine preferred genes for use in some embodiments of the disclosure. Rankings of select BCRGs, TCRGs, HLAGs and OCPGs according to their correlation with the mean cluster expression as described in the Examples below are given in Tables 28, 29, 30, and 31 below as well as their ranking according to predictive value are given in Tables 6, 8, and 9.

Thus, in some embodiments of each of the various aspects of the disclosure the plurality of test genes comprises at least some number of genes selected from BCRGs, TCRGs, HLAGs and OCPGs (e.g., at least 2, 3, 4, 5, 6, 7, 8, 9, 10, 15, 20, 25, 30, 35, 40, 45, 50 or more genes selected from BCRGs, TCRGs, HLAGs and OCPGs). In some embodiments the plurality of test genes comprises the top 2, 3, 4, 5, 6, 7, 8, 9, 10, or all 11 genes selected from BCRGs, TCRGs, HLAGs and OCPGs listed in Table 30. In some embodiments the plurality of test genes comprises the top 2, 3, 4, 5, 6, 7, 8, 9, 10, or all 11 genes selected from BCRGs, TCRGs, HLAGs and OCPGs listed in Table 28. In some embodiments the plurality of test genes comprises at least some number of genes selected from BCRGs, TCRGs, HLAGs and OCPGs (e.g., at least 2, 3, 4, 5, 6, 7, 8, 9, 10, 15, 20, 25, 30, 35, 40, 45, 50 or more genes selected from BCRGs, TCRGs, HLAGs and OCPGs) and this plurality of genes selected from BCRGs, TCRGs, HLAGs and OCPGs comprises at least 1, 2, 3, 4, 5, 6, 7, 8, 9, 10, or 14 of the following genes: IRF4, CCL19, SELL, CD38, CCL5, IGLL5/CKAP2, CCR2, TRDV3/TRDV1, IGHM, IGJ, or PTRPC. In some embodiments the plurality of test genes comprises the top 2, 3, 4, 5, 6, 7, 8, 9, 10, 11, 12, 13, or all 14 genes selected from BCRGs, TCRGs, HLAGs and OCPGs listed in Table 31. In some embodiments the plurality of test genes comprises at least some number of genes selected from BCRGs, TCRGs, HLAGs and OCPGs (e.g., at least 2, 3, 4, 5, 6, 7, 8, 9, 10, 15, 20, 25, 30, 35, 40, 45, 50 or more genes selected from BCRGs, TCRGs, HLAGs and OCPGs) and this plurality of genes selected from BCRGs, TCRGs, HLAGs and OCPGs comprises at least 1, 2, 3, 4, 5, 6, 7, 8, 9, 10, or 14 of the following genes: ITGB2, EVI2B, HCLS1, HLA-DPB1, HLA-E, HLA-DPA1, HLA-DRA, HLA-DMA, PECAM1, EVI2B, PTPN22, IRF1, CD74, or, HLA-DRB1. In some embodiments the plurality of test genes comprises the top 2, 3, 4, 5, 6, 7, 8, 9, 10, 11, 12, 13, or all 14 genes selected from BCRGs, TCRGs, HLAGs and OCPGs listed in Table 32. In some embodiments the plurality of test genes comprises at least some number of genes selected from BCRGs, TCRGs, HLAGs and OCPGs (e.g., at least 2, 3, 4, 5, 6, 7, 8, 9, 10, 15, 20, 25, 30, 35, 40, 45, 50 or more genes selected from BCRGs, TCRGs, HLAGs and OCPGs) and this plurality of genes selected from BCRGs, TCRGs, HLAGs and OCPGs comprises at least 1, 2, 3, 4, 5, 6, 7, 8, 9, 10, or 14 of the following genes: IRF4, CD38, SELL, CCL5, IGHM, IGLL5/CKAP2, PTPRC, IGH, EVI2B, CCL19, TRDV3/TRDV1, PTPN22, or, PECAM1, In some embodiments the plurality of test genes comprises the top 2, 3, 4, 5, 6, 7, 8, or all 9 genes selected from BCRGs, TCRGs, HLAGs and OCPGs listed in Table 33. In some embodiments the plurality of test genes comprises at least some number of genes selected from BCRGs, TCRGs, HLAGs and OCPGs (e.g., at least 2, 3, 4, 5, 6, 7, 8, 9, 10, 15, 20, 25, 30, 35, 40, 45, 50 or more genes selected from BCRGs, TCRGs, HLAGs and OCPGs) and this plurality of genes selected from BCRGs, TCRGs, HLAGs and OCPGs comprises at least 1, 2, 3, 4, 5, 6, 7, 8, or, 9 of the following genes: HLA-DMA, HLA-DPB1, HLA-DRA, HLA-E, HLA-DPA1, HCLS1, ITGB2, HLA-DRB3, or, HLA-DRB3/HLA-DRB1.

Assays of the BCRGs, TCRGs, HLAGs and OCPGs as described in Example 2 and 3 below were run against 47 and 71 ER+ breast tumor samples, respectively, commercially obtained (anonymous tumor FFPE samples without outcome or other clinical data). The working hypothesis was that the assays would measure with varying degrees of accuracy the same underlying phenomenon. Assays were ranked by the Pearson's correlation coefficient between the individual gene and the mean of all the particular genes as described in more detail below, that being the best available estimate of relevance. Rankings for these genes according to their correlation to their respective cluster means are reported in Tables 30, 31, 32, or 33 below in Examples 2 and 3.

When choosing specific BCRGs, TCRGs, HLAGs, or OCPGs for inclusion in any embodiment of the disclosure, the individual predictive power of each gene may be used to rank them in importance. The inventors have determined that the BCRGs, TCRGs, HLAGs, or OCPGs (or the indicated probes) can be ranked as shown in Table 6A and 6B above according to the predictive power of each individual gene. Further, a subset of the ISGs and OCPGs of the disclosure (Immune Panel 3) can be ranked according to Univariate and multivariate p-value as shown in Tables 8 and 9 below.

TABLE 8

| | | | Univariate |
|---|---|---|---|
| Gene # | Gene | Identifier | p-value |
| 1 | IGJ | Hs00950678_g1 | 1.10E−07 |
| 2 | HCLS1 | Hs00945386_m1 | 3.90E−03 |
| 3 | CCL19 | Hs00171149_m1 | 5.80E−03 |
| 4 | EVI2B | Hs00272421_s1 | 7.20E−03 |
| 5 | CCL5 | Hs00174575_m1 | 4.00E−02 |
| 6 | PTPRC | Hs00894732_m1 | 5.80E−02 |
| 7 | IRF1 | Hs00971965_m1 | 6.10E−01 |

TABLE 9

| Gene | | | Multivariate |
|---|---|---|---|
| # | Gene | Identifier | p-value |
| 1 | IGJ | Hs00950678_g1 | 2.80E−05 |
| 2 | EVI2B | Hs00272421_s1 | 4.80E−03 |
| 3 | CCL19 | Hs00171149_m1 | 6.50E−03 |
| 4 | HCLS1 | Hs00945386_m1 | 1.30E−02 |
| 5 | CCL5 | Hs00174575_m1 | 3.90E−02 |
| 6 | PTPRC | Hs00894732_m1 | 1.20E−01 |
| 7 | IRF1 | Hs00971965_m1 | 3.90E−01 |

Thus, in some embodiments of each of the various aspects of the disclosure the plurality of test genes comprises at least some number of genes selected from BCRGs, TCRGs, HLAGs and OCPGs (e.g., at least 2, 3, 4, 5, 6, 7, 8, 9, 10, 15, 20, 25, 30, 35, 40, 45, 50 or more genes selected from BCRGs, TCRGs, HLAGs and OCPGs). In some embodiments the plurality of test genes comprises the top 2, 3, 4, 5, 6, 7, 8, 9, 10, 11, 12, 13, 14, 15, 20, 25, 30, 35, 40 or more genes selected from BCRGs, TCRGs, HLAGs and OCPGs listed in any of Tables 1, 6A, 6B, 8, 9, 30, 31, 32, or 33. In some embodiments the plurality of test genes comprises at least some number of genes selected from BCRGs, TCRGs, HLAGs and OCPGs (e.g., at least 2, 3, 4, 5, 6, 7, 8, 9, 10, 15, 20, 25, 30, 35, 40, 45, 50 or more genes selected from BCRGs, TCRGs, HLAGs and OCPGs) and this plurality of genes selected from BCRGs, TCRGs, HLAGs and OCPGs comprises at least 1, 2, 3, 4, 5, 6, 7, 8, 9, 10, 15, or 20 of the following genes: CKAP2, GUSBP11, IGHM, IGJ, IGkappa, IGKC, IGKV1-5, IGL1, IGLL3P, IGVH, CCL19, CCL5, CCR2, CD247, CD38, HLA-E, IRF1, IRF4, PTPN22, SELL, SEMA4D, TCRA/D, CD74, EVI2B, HCLS1, HLA-DMA, HLA-DPA1, HLA-DPB1, HLA-DQB1, HLA-DRA, HLA-DRB1, HLA-DRB1/3, ITGB2, PECAM1, PTPRC, ABCC5, APOBEC3F, ARID5B, C3, CACNB3, CALD1, CEP57, CNOT2, CPT1A, CTTN, CXCL12, DLAT, EPB41L2, ERP29, FTH1, GPRC5A, HSD11B1, LGR4, LITAF, LPPR2, MCF2L, NECAP2, NHLH2, NTM, PCDH12, PCDH17, PDGFB, POLR2H, PPFIA1, RAC2, RACGAP1, RBM7, RFK, RPA2, RPL5, SIX1, SIX2, SLC35E3, SLC4A8, SRRM1, STAT5A, TPD52, XPO7, and ZFP36L2. In some embodiments the plurality of test genes comprises at least some number of genes selected from BCRGs, TCRGs, HLAGs and OCPGs (e.g., at least 3, 4, 5, 6, 7, 8, 9, 10, 15, 20, 25, 30, 35, 40, 45, 50 or more genes selected from BCRGs, TCRGs, HLAGs and OCPGs) and this plurality of test genes comprises any one, two, three, four, five, six, seven, eight, nine, or ten or all of gene numbers 1, 1& 2, 1 to 3, 1 to 4, 1 to 5, 1 to 6, 1 to 7, 1 to 8, 1 to 9, or 1 to 10 of any of Tables 1, 6A, 6B, 8, 9, 30, 31, 32, or 33. In some embodiments the plurality of test genes comprises at least some number of genes selected from BCRGs, TCRGs, HLAGs and OCPGs (e.g., at least 3, 4, 5, 6, 7, 8, 9, 10, 15, 20, 25, 30, 35, 40, 45, 50 or more genes selected from BCRGs, TCRGs, HLAGs and OCPGs) and this plurality of genes selected from BCRGs, TCRGs, HLAGs and OCPGs comprises any one, two, three, four, five, six, seven, eight, or nine or all of gene numbers 2, 2 & 3, 2 to 4, 2 to 5, 2 to 6, 2 to 7, 2 to 8, 2 to 9, or 2 to 10 of any of Tables 1, 6A, 6B, 8, 9, 30, 31, 32, or 33. In some embodiments the plurality of test genes comprises at least some number of genes selected from BCRGs, TCRGs, HLAGs and OCPGs (e.g., at least 3, 4, 5, 6, 7, 8, 9, 10, 15, 20, 25, 30, 35, 40, 45, 50 or more genes selected from BCRGs, TCRGs, HLAGs and OCPGs) and this plurality of genes selected from BCRGs, TCRGs, HLAGs and OCPGs comprises any one, two, three, four, five, six, seven, or eight or all of gene numbers 3, 3 & 4, 3 to 5, 3 to 6, 3 to 7, 3 to 8, 3 to 9, or 3 to 10 of any of Tables 1, 6A, 6B, 8, 9, 30, 31, 32, or 33. In some embodiments the plurality of test genes comprises at least some number of genes selected from BCRGs, TCRGs, HLAGs and OCPGs (e.g., at least 3, 4, 5, 6, 7, 8, 9, 10, 15, 20, 25, 30, 35, 40, 45, 50 or more genes selected from BCRGs, TCRGs, HLAGs and OCPGs) and this plurality of genes selected from BCRGs, TCRGs, HLAGs and OCPGs comprises any one, two, three, four, five, six, or seven or all of gene numbers 4, 4 & 5, 4 to 6, 4 to 7, 4 to 8, 4 to 9, or 4 to 10 of any of Tables 1, 6A, 6B, 8, 9, 30, 31, 32, or 33. In some embodiments the plurality of test genes comprises at least some number of genes selected from BCRGs, TCRGs, HLAGs and OCPGs (e.g., at least 3, 4, 5, 6, 7, 8, 9, 10, 15, 20, 25, 30, 35, 40, 45, 50 or more genes selected from BCRGs, TCRGs, HLAGs and OCPGs) and this plurality of genes selected from BCRGs, TCRGs, HLAGs and OCPGs comprises any one, two, three, four, five, six, seven, eight, nine, 10, 11, 12, 13, 14, or 15 or all of gene numbers 1, 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Tables 1, 6A, 6B, 8, 9, 30, 31, 32, or 33.

In a previous study (International Application No. PCT/US2011/049760 (published as WO/2012/030840), incorporated herein in its entirety by reference) 126 CCGs and 47 housekeeping genes had their expression compared to the CCG and housekeeping mean in order to determine preferred genes for use in some embodiments of the disclosure. Rankings of select CCGs according to their correlation with the mean CCG expression as well as their ranking according to predictive value are given in, e.g., Tables 10, 11, 12, 13, and 14. According to some embodiments or aspects of the disclosure, the methods and compositions include CCGs as described in more detail below.

Assays of 126 CCGs and 47 HK (housekeeping) genes were run against 96 commercially obtained, anonymous tumor FFPE samples without outcome or other clinical data. The working hypothesis was that the assays would measure with varying degrees of accuracy the same underlying phenomenon (cell cycle proliferation within the tumor for the CCGs, and sample concentration for the HK genes). Assays were ranked by the Pearson's correlation coefficient between the individual gene and the mean of all the candidate genes, that being the best available estimate of biological activity. Rankings for these 126 CCGs according to their correlation to the overall CCG mean are reported in Table 10.

TABLE 10

| Gene # | Gene Symbol | Correl. w/Mean |
|---|---|---|
| 1 | TPX2 | 0.931 |
| 2 | CCNB2 | 0.9287 |
| 3 | KIF4A | 0.9163 |
| 4 | KIF2C | 0.9147 |
| 5 | BIRC5 | 0.9077 |
| 6 | BIRC5 | 0.9077 |
| 7 | RACGAP1 | 0.9073 |
| 8 | CDC2 | 0.906 |
| 9 | PRC1 | 0.9053 |
| 10 | DLGAP5/DLG7 | 0.9033 |
| 11 | CEP55 | 0.903 |
| 12 | CCNB1 | 0.9 |
| 13 | TOP2A | 0.8967 |
| 14 | CDC20 | 0.8953 |
| 15 | KIF20A | 0.8927 |
| 16 | BUB1B | 0.8927 |
| 17 | CDKN3 | 0.8887 |
| 18 | NUSAP1 | 0.8873 |
| 19 | CCNA2 | 0.8853 |
| 20 | KIF11 | 0.8723 |
| 21 | CDCA8 | 0.8713 |
| 22 | NCAPG | 0.8707 |
| 23 | ASPM | 0.8703 |
| 24 | FOXM1 | 0.87 |
| 25 | NEK2 | 0.869 |
| 26 | ZWINT | 0.8683 |
| 27 | PTTG1 | 0.8647 |
| 28 | RRM2 | 0.8557 |
| 29 | TTK | 0.8483 |
| 30 | TRIP13 | 0.841 |
| 31 | GINS1 | 0.841 |
| 32 | CENPF | 0.8397 |
| 33 | HMMR | 0.8367 |
| 34 | NCAPH | 0.8353 |
| 35 | NDC80 | 0.8313 |
| 36 | KIF15 | 0.8307 |
| 37 | CENPE | 0.8287 |
| 38 | TYMS | 0.8283 |
| 39 | KIAA0101 | 0.8203 |
| 40 | FANCI | 0.813 |
| 41 | RAD51AP1 | 0.8107 |
| 42 | CKS2 | 0.81 |
| 43 | MCM2 | 0.8063 |
| 44 | PBK | 0.805 |
| 45 | ESPL1 | 0.805 |
| 46 | MKI67 | 0.7993 |
| 47 | SPAG5 | 0.7993 |
| 48 | MCM10 | 0.7963 |
| 49 | MCM6 | 0.7957 |
| 50 | OIP5 | 0.7943 |
| 51 | CDC45L | 0.7937 |
| 52 | KIF23 | 0.7927 |
| 53 | EZH2 | 0.789 |
| 54 | SPC25 | 0.7887 |
| 55 | STIL | 0.7843 |
| 56 | CENPN | 0.783 |
| 57 | GTSE1 | 0.7793 |
| 58 | RAD51 | 0.779 |
| 59 | CDCA3 | 0.7783 |
| 60 | TACC3 | 0.778 |
| 61 | PLK4 | 0.7753 |
| 62 | ASF1B | 0.7733 |
| 63 | DTL | 0.769 |
| 64 | CHEK1 | 0.7673 |
| 65 | NCAPG2 | 0.7667 |
| 66 | PLK1 | 0.7657 |
| 67 | TIMELESS | 0.762 |
| 68 | E2F8 | 0.7587 |
| 69 | EXO1 | 0.758 |
| 70 | ECT2 | 0.744 |
| 71 | STMN1 | 0.737 |
| 72 | STMN1 | 0.737 |
| 73 | RFC4 | 0.737 |
| 74 | CDC6 | 0.7363 |
| 75 | CENPM | 0.7267 |
| 76 | MYBL2 | 0.725 |
| 77 | SHCBP1 | 0.723 |
| 78 | ATAD2 | 0.723 |
| 79 | KIFC1 | 0.7183 |
| 80 | DBF4 | 0.718 |
| 81 | CKS1B | 0.712 |
| 82 | PCNA | 0.7103 |
| 83 | FBXO5 | 0.7053 |
| 84 | C12orf48 | 0.7027 |
| 85 | TK1 | 0.7017 |
| 86 | BLM | 0.701 |
| 87 | KIF18A | 0.6987 |
| 88 | DONSON | 0.688 |
| 89 | MCM4 | 0.686 |
| 90 | RAD54B | 0.679 |
| 91 | RNASEH2A | 0.6733 |
| 92 | TUBA1C | 0.6697 |
| 93 | C18orf24 | 0.6697 |
| 94 | SMC2 | 0.6697 |
| 95 | CENPI | 0.6697 |
| 96 | GMPS | 0.6683 |
| 97 | DDX39 | 0.6673 |
| 98 | POLE2 | 0.6583 |
| 99 | APOBEC3B | 0.6513 |
| 100 | RFC2 | 0.648 |
| 101 | PSMA7 | 0.6473 |
| 102 | MPHOSPH1/kif20b | 0.6457 |
| 103 | CDT1 | 0.645 |
| 104 | H2AFX | 0.6387 |
| 105 | ORC6L | 0.634 |
| 106 | C1orf135 | 0.6333 |
| 107 | PSRC1 | 0.633 |
| 108 | VRK1 | 0.6323 |
| 109 | CKAP2 | 0.6307 |
| 110 | CCDC99 | 0.6303 |
| 111 | CCNE1 | 0.6283 |
| 112 | LMNB2 | 0.625 |
| 113 | GPSM2 | 0.625 |
| 114 | PAICS | 0.6243 |
| 115 | MCAM | 0.6227 |
| 116 | DSN1 | 0.622 |
| 117 | NCAPD2 | 0.6213 |
| 118 | RAD54L | 0.6213 |
| 119 | PDSS1 | 0.6203 |
| 120 | HN1 | 0.62 |
| 121 | C21orf45 | 0.6193 |
| 122 | CTSL2 | 0.619 |
| 123 | CTPS | 0.6183 |
| 124 | MCM7 | 0.618 |
| 125 | ZWILCH | 0.618 |
| 126 | RFC5 | 0.6177 |

After excluding CCGs with low average expression, assays that produced sample failures, CCGs with correlations less than 0.58, and HK genes with correlations less than 0.95, a subset of 56 CCGs (Panel G) and 36 HK candidate genes were left. Correlation coefficients were recalculated on these subsets, with the rankings shown in Table 11 and Table B, respectively.

TABLE 11
(“Panel G”)

| Gene # | Gene Symbol | Correl. w/CCG mean |
|---|---|---|
| 1 | FOXM1 | 0.908 |
| 2 | CDC20 | 0.907 |
| 3 | CDKN3 | 0.9 |
| 4 | CDC2 | 0.899 |
| 5 | KIF11 | 0.898 |
| 6 | KIAA0101 | 0.89 |
| 7 | NUSAP1 | 0.887 |
| 8 | CENPF | 0.882 |
| 9 | ASPM | 0.879 |
| 10 | BUB1B | 0.879 |
| 11 | RRM2 | 0.876 |
| 12 | DLGAP5 | 0.875 |
| 13 | BIRC5 | 0.864 |
| 14 | KIF20A | 0.86 |
| 15 | PLK1 | 0.86 |
| 16 | TOP2A | 0.851 |
| 17 | TK1 | 0.837 |
| 18 | PBK | 0.831 |
| 19 | ASF1B | 0.827 |
| 20 | C18orf24 | 0.817 |
| 21 | RAD54L | 0.816 |
| 22 | PTTG1 | 0.814 |
| 23 | KIF4A | 0.814 |
| 24 | CDCA3 | 0.811 |
| 25 | MCM10 | 0.802 |
| 26 | PRC1 | 0.79 |
| 27 | DTL | 0.788 |
| 28 | CEP55 | 0.787 |
| 29 | RAD51 | 0.783 |
| 30 | CENPM | 0.781 |
| 31 | CDCA8 | 0.774 |
| 32 | OIP5 | 0.773 |
| 33 | SHCBP1 | 0.762 |
| 34 | ORC6L | 0.736 |
| 35 | CCNB1 | 0.727 |
| 36 | CHEK1 | 0.723 |
| 37 | TACC3 | 0.722 |
| 38 | MCM4 | 0.703 |
| 39 | FANCI | 0.702 |
| 40 | KIF15 | 0.701 |
| 41 | PLK4 | 0.688 |
| 42 | APOBEC3B | 0.67 |
| 43 | NCAPG | 0.667 |
| 44 | TRIP13 | 0.653 |
| 45 | KIF23 | 0.652 |
| 46 | NCAPH | 0.649 |
| 47 | TYMS | 0.648 |
| 48 | GINS1 | 0.639 |
| 49 | STMN1 | 0.63 |
| 50 | ZWINT | 0.621 |
| 51 | BLM | 0.62 |
| 52 | TTK | 0.62 |
| 53 | CDC6 | 0.619 |
| 54 | KIF2C | 0.596 |
| 55 | RAD51AP1 | 0.567 |
| 56 | NCAPG2 | 0.535 |

TABLE B

| | | Correlation |
|---|---|---|
| Gene | Gene | with HK |
| # | Symbol | Mean |
| 1 | RPL38 | 0.989 |
| 2 | UBA52 | 0.986 |
| 3 | PSMC1 | 0.985 |
| 4 | RPL4 | 0.984 |
| 5 | RPL37 | 0.983 |
| 6 | RP529 | 0.983 |
| 7 | SLC25A3 | 0.982 |
| 8 | CLTC | 0.981 |
| 9 | TXNL1 | 0.98 |
| 10 | PSMA1 | 0.98 |
| 11 | RPL8 | 0.98 |
| 12 | MMADHC | 0.979 |
| 13 | RPL13A; | 0.979 |
| | LOC728658 | |
| 14 | PPP2CA | 0.978 |
| 15 | MRFAP1 | 0.978 |

The CCGs in Panel F were likewise ranked according to correlation to the CCG mean as shown in Table 12 below.

TABLE 12

| Gene # | Gene Symbol | Correl. w/CCG mean |
|---|---|---|
| 1 | DLGAP5 | 0.931 |
| 2 | ASPM | 0.931 |
| 3 | KIF11 | 0.926 |
| 4 | BIRC5 | 0.916 |
| 5 | CDCA8 | 0.902 |
| 6 | CDC20 | 0.9 |
| 7 | MCM10 | 0.899 |
| 8 | PRC1 | 0.895 |
| 9 | BUB1B | 0.892 |
| 10 | FOXM1 | 0.889 |
| 11 | NUSAP1 | 0.888 |
| 12 | C18orf24 | 0.885 |
| 13 | PLK1 | 0.879 |
| 14 | CDKN3 | 0.874 |
| 15 | RRM2 | 0.871 |
| 16 | RAD51 | 0.864 |
| 17 | CEP55 | 0.862 |
| 18 | ORC6L | 0.86 |
| 19 | RAD54L | 0.86 |
| 20 | CDC2 | 0.858 |
| 21 | CENPF | 0.855 |
| 22 | TOP2A | 0.852 |
| 23 | KIF20A | 0.851 |
| 24 | KIAA0101 | 0.839 |
| 25 | CDCA3 | 0.835 |
| 26 | ASF1B | 0.797 |
| 27 | CENPM | 0.786 |
| 28 | TK1 | 0.783 |
| 29 | PBK | 0.775 |
| 30 | PTTG1 | 0.751 |
| 31 | DTL | 0.737 |

When choosing specific CCGs for inclusion in any embodiment of the disclosure, the individual predictive power of each gene may be used to rank them in importance. The inventors have determined that the CCGs in Panel C can be ranked as shown in Table 13 below according to the predictive power of each individual gene. The CCGs in Panel F can be similarly ranked as shown in Table 14 below.

TABLE 13

| Gene # | Gene | p-value |
|---|---|---|
| 1 | NUSAP1 | 2.8E−07 |
| 2 | DLG7 | 5.9E−07 |
| 3 | CDC2 | 6.0E−07 |
| 4 | FOXM1 | 1.1E−06 |
| 5 | MYBL2 | 1.1E−06 |
| 6 | CDCA8 | 3.3E−06 |
| 7 | CDC20 | 3.8E−06 |
| 8 | RRM2 | 7.2E−06 |
| 9 | PTTG1 | 1.8E−05 |
| 10 | CCNB2 | 5.2E−05 |
| 11 | HMMR | 5.2E−05 |
| 12 | BUB1 | 8.3E−05 |
| 13 | PBK | 1.2E−04 |
| 14 | TTK | 3.2E−04 |
| 15 | CDC45L | 7.7E−04 |
| 16 | PRC1 | 1.2E−03 |
| 17 | DTL | 1.4E−03 |
| 18 | CCNB1 | 1.5E−03 |
| 19 | TPX2 | 1.9E−03 |
| 20 | ZWINT | 9.3E−03 |
| 21 | KIF23 | 1.1E−02 |
| 22 | TRIP13 | 1.7E−02 |
| 23 | KPNA2 | 2.0E−02 |
| 24 | UBE2C | 2.2E−02 |
| 25 | MELK | 2.5E−02 |
| 26 | CENPA | 2.9E−02 |
| 27 | CKS2 | 5.7E−02 |
| 28 | MAD2L1 | 1.7E−01 |
| 29 | UBE2S | 2.0E−01 |
| 30 | AURKA | 4.8E−01 |
| 31 | TIMELESS | 4.8E−01 |

TABLE 14

| Gene # | Gene Symbol | p-value |
|---|---|---|
| 1 | MCM10 | 8.60E−10 |
| 2 | ASPM | 2.30E−09 |
| 3 | DLGAP5 | 1.20E−08 |
| 4 | CENPF | 1.40E−08 |
| 5 | CDC20 | 2.10E−08 |
| 6 | FOXM1 | 3.40E−07 |
| 7 | TOP2A | 4.30E−07 |
| 8 | NUSAP1 | 4.70E−07 |
| 9 | CDKN3 | 5.50E−07 |
| 10 | KIF11 | 6.30E−06 |
| 11 | KIF20A | 6.50E−06 |
| 12 | BUB1B | 1.10E−05 |
| 13 | RAD54L | 1.40E−05 |
| 14 | CEP55 | 2.60E−05 |
| 15 | CDCA8 | 3.10E−05 |
| 16 | TK1 | 3.30E−05 |
| 17 | DTL | 3.60E−05 |
| 18 | PRC1 | 3.90E−05 |
| 19 | PTTG1 | 4.10E−05 |
| 20 | CDC2 | 0.00013 |
| 21 | ORC6L | 0.00017 |
| 22 | PLK1 | 0.0005 |
| 23 | C18orf24 | 0.0011 |
| 24 | BIRC5 | 0.00118 |
| 25 | RRM2 | 0.00255 |
| 26 | CENPM | 0.0027 |
| 27 | RAD51 | 0.0028 |
| 28 | KIAA0101 | 0.00348 |
| 29 | CDCA3 | 0.00863 |
| 30 | PBK | 0.00923 |
| 31 | ASF1B | 0.00936 |

Thus, in some embodiments of each of the various aspects of the disclosure the plurality of test genes, in addition to a plurality (e.g., at least 2, 4, 6, 8, 10, or 12 or more) of the BCRGs, TCRGs, HLAGs, and OCPGs as described herein, comprises the top 2, 3, 4, 5, 6, 7, 8, 9, 10, 11, 12, 13, 14, 15, 20, 25, 30, 35, 40 or more CCGs listed in any of Tables 10, 11, 12, 13, 14, 15, 19, 20, 21, 22, 23, 24, 25, 34, or 35. In some embodiments the plurality of test genes, in addition to at least 2, 4, 6, 8, 10, or 12 or more of the BCRGs, TCRGs, HLAGs, and OCPGS as described herein, comprises at least some number of CCGs (e.g., at least 3, 4, 5, 6, 7, 8, 9, 10, 15, 20, 25, 30, 35, 40, 45, 50 or more CCGs) and this plurality of CCGs comprises at least 1, 2, 3, 4, 5, 6, 7, 8, 9, 10, 15, or 20 of the following genes: ASPM, BIRC5, BUB1B, CCNB2, CDC2, CDC20, CDCAB, CDKN3, CENPF, DLGAP5, FOXM1, KIAA0101, KIF11, KIF2C, KIF4A, MCM10, NUSAP1, PRC1, RACGAP1, and TPX2. In some embodiments the plurality of test genes, in addition to at least 2, 4, 6, 8, 10, or 12 or more of the BCRGs, TCRGs, HLAGs, and OCPGs as described herein, comprises at least some number of CCGs (e.g., at least 3, 4, 5, 6, 7, 8, 9, 10, 15, 20, 25, 30, 35, 40, 45, 50 or more CCGs) and this plurality of CCGs comprises at least 1, 2, 3, 4, 5, 6, 7, 8, 9, 10, 15, or 20 of the following genes: TPX2, CCNB2, KIF4A, KIF2C, BIRC5, RACGAP1, CDC2, PRC1, DLGAP5/DLG7, CEP55, CCNB1, TOP2A, CDC20, KIF20A, BUB1B, CDKN3, NUSAP1, CCNA2, KIF11, and CDCAB. In some embodiments the plurality of test genes, in addition to at least 2, 4, 6, 8, 10, or 12 or more of the BCRGs, TCRGs, HLAGs, and OCPGs as described herein, comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, nine, or ten or all of gene numbers 1, 1& 2, 1 to 3, 1 to 4, 1 to 5, 1 to 6, 1 to 7, 1 to 8, 1 to 9, or 1 to 10 of any of Tables 10, 11, 12, 13, 14, 15, 19, 20, 21, 22, 23, 24, 25, 34, or 35. In some embodiments the plurality of test genes, in addition to at least 2, 4, 6, 8, 10, or 12 or more of the BCRGs, TCRGs, HLAGs, and OCPGs as described herein, comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, or nine or all of gene numbers 2, 2 & 3, 2 to 4, 2 to 5, 2 to 6, 2 to 7, 2 to 8, 2 to 9, or 2 to 10 of any of Tables 10, 11, 12, 13, 14, 15, 19, 20, 21, 22, 23, 24, 25, 34, or 35. In some embodiments the plurality of test genes, in addition to at least 2, 4, 6, 8, 10, or 12 or more of the BCRGs, TCRGs, HLAGs, and OCPGs as described herein, comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, or eight or all of gene numbers 3, 3 & 4, 3 to 5, 3 to 6, 3 to 7, 3 to 8, 3 to 9, or 3 to 10 of any of Tables 10, 11, 12, 13, 14, 15, 19, 20, 21, 22, 23, 24, 25, 34, or 35. In some embodiments the plurality of test genes, in addition to at least 2, 4, 6, 8, 10, or 12 or more of the BCRGs, TCRGs, HLAGs, and OCPGs as described herein, comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, or seven or all of gene numbers 4, 4 & 5, 4 to 6, 4 to 7, 4 to 8, 4 to 9, or 4 to 10 of any of Tables 10, 11, 12, 13, 14, 15, 19, 20, 21, 22, 23, 24, 25, 34, or 35. In some embodiments the plurality of test genes, in addition to at least 2, 4, 6, 8, 10, or 12 or more of the BCRGs, TCRGs, HLAGs, and OCPGs as described herein, comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, nine, 10, 11, 12, 13, 14, or 15 or all of gene numbers 1, 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Tables 10, 11, 12, 13, 14, 15, 19, 20, 21, 22, 23, 24, 25, 34, or 35.

In preferred embodiments, the test value representing the overall expression of the plurality of test genes is compared to one or more reference values (or index values), and optionally correlated to breast cancer prognosis, or an increased or no increased likelihood of breast cancer recurrence or post-surgery metastasis-free survival. In some embodiments a test value greater than the reference value(s) can be correlated to increased likelihood of poor prognosis or decreased probability of post-surgery metastasis-free survival. In some embodiments the test value is deemed “greater than” the reference value (e.g., the threshold index value), and thus correlated to an increased likelihood of poor prognosis or decreased probability of post-surgery metastasis-free survival, if the test value exceeds the reference value by at least some amount (e.g., at least 0.5, 0.75, 0.85, 0.90, 0.95, 1, 2, 3, 4, 5, 6, 7, 8, 9, or 10 or more fold or standard deviations).

For example, the index value may represent the gene expression levels found in a normal sample obtained from the patient of interest (including tissue surrounding the cancerous tissue in a biopsy), in which case an expression level in the sample significantly higher than this index value would indicate, e.g., increased likelihood of response to a particular treatment regimen (e.g., a treatment regimen comprising chemotherapy).

Alternatively, the index value may represent the average expression level for a set of individuals from a diverse cancer population or a subset of the population. For example, one may determine the average expression level of a gene or gene panel in a random sampling of patients with cancer (e.g., breast cancer). This average expression level may be termed the “threshold index value”.

Alternatively, the index value may represent the average expression level of a particular gene or gene panel in a plurality of training patients (e.g., breast cancer patients) with similar outcomes whose clinical and follow-up data are available and sufficient to define and categorize the patients by disease outcome. See, e.g., Examples, infra. For example, a “good prognosis index value” can be generated from a plurality of training cancer patients characterized as having “good prognosis” after breast cancer surgery and hormone deprivation therapy. A “poor prognosis index value” can be generated from a plurality of training cancer patients defined as having “poor prognosis” breast cancer surgery and hormone deprivation therapy. Thus, a good prognosis index value of a particular gene or gene panel may represent the average level of expression of the particular gene or gene panel in patients having a “good prognosis,” whereas a poor prognosis index value of a particular gene or gene panel represents the average level of expression of the particular gene or gene panel in patients having a “poor prognosis.” Thus, if the determined level of expression of a relevant gene or gene panel is closer to the good prognosis index value of the gene or gene panel than to the poor prognosis index value of the gene or gene panel, then it can be concluded that the patient is more likely to have a good prognosis. On the other hand, if the determined level of expression of a relevant gene or gene panel is closer to the poor prognosis index value of the gene or gene panel than to the good prognosis index value of the gene or gene panel, then it can be concluded that the patient is more likely to have a poor prognosis.

Alternatively index values may be determined thusly: In order to assign patients to risk groups, a threshold value may be set for the cell cycle mean combined with the ABCC5 mean, and optionally PGR mean. The optimal threshold value is selected based on the receiver operating characteristic (ROC) curve, which plots sensitivity vs (1−specificity). For each increment of the combined mean, the sensitivity and specificity of the test is calculated using that value as a threshold. The actual threshold will be the value that optimizes these metrics according to the artisan's requirements (e.g., what degree of sensitivity or specificity is desired, etc.).

Those skilled in the art are familiar with various ways of determining the expression of a panel of genes (i.e., a plurality of genes). One may determine the expression of a panel of genes by determining the average expression level (normalized or absolute) of all panel genes in a sample obtained from a particular patient (either throughout the sample or in a subset of cells or a single cell from the sample). Increased expression in this context will mean the average expression is higher than the average expression level of these genes in some reference (e.g., higher than in normal patients; higher than some index value that has been determined to represent the average expression level in a reference population, such as patients with the same cancer; etc.). Alternatively, one may determine the expression of a panel of genes by determining the average expression level (normalized or absolute) of at least a certain number (e.g., 1, 2, 3, 4, 5, 6, 7, 8, 9, 10, 15, 20, 25, 30 or more) or at least a certain proportion (e.g., 10%, 20%, 30%, 40%, 50%, 60%, 70%, 80%, 90%, 95%, 99%, 100%) of the genes in the panel. Alternatively, one may determine the expression of a panel of genes by determining the absolute copy number of the analyte representing each gene in the panel (e.g., mRNA, cDNA, protein) and either total or average these across the genes.

Panels of genes selected from BCRGs, TCRGs, HLAGs and OCPGs, alone or in combination with CCGs (e.g., 2, 3, 4, 5, or 6 CCGs) can accurately predict cancer prognosis, and in particular breast cancer prognosis. But addition of the ABCC5 and PGR genes significantly increases the prediction power. In some embodiments the panel comprises at least 3, 4, 5, 6, 7, 8, 9, 10, 15, 20, 25, 30, 35, 40, 45, 50, 70, 80, 90, 100, 200, or more genes selected from BCRGs, TCRGs, HLAGs, OCPGs, and CCGs. In some embodiments the panel comprises the ABCC5 and PGR genes and at least 2, 3, 4, 5, 6, 7, 8, 9, 10, 15, 20, 25, 30, 35, 40, 45, 50, 70, 80, 90, 100, 200, or more genes selected from BCRGs, TCRGs, HLAGs, OCPGs and CCGs. In some embodiments the panel comprises at least 10, 15, 20, or more genes selected from BCRGs, TCRGs, HLAGs, OCPGs, and CCGs. In some embodiments the panel comprises between 5 and 100 genes selected from BCRGs, TCRGs, HLAGs, OCPGs, and CCGs, between 7 and 40 genes selected from BCRGs, TCRGs, HLAGs, OCPGs, and CCGs, between 5 and 25 genes selected from BCRGs, TCRGs, HLAGs, OCPGs, and CCGs, between 10 and 20 genes selected from BCRGs, TCRGs, HLAGs, OCPGs, and CCGs, or between 10 and 15 genes selected from BCRGs, TCRGs, HLAGs, OCPGs, and CCGs. In some embodiments the genes selected from BCRGs, TCRGs, HLAGs, OCPGs, and CCGs comprise at least a certain proportion of the panel. Thus, in some embodiments the panel comprises at least 25%, 30%, 40%, 50%, 60%, 70%, 75%, 80%, 85%, 90%, 95%, 96%, 97%, 98%, or 99% genes selected from BCRGs, TCRGs, HLAGs, OCPGs, and CCGs. In some preferred embodiments the panel comprises at least 10, 15, 20, 25, 30, 35, 40, 45, 50, 70, 80, 90, 100, 200, or more genes selected from BCRGs, TCRGs, HLAGs, OCPGs, and CCGs, and such genes selected from BCRGs, TCRGs, HLAGs, OCPGs, and CCGs constitute of at least 50%, 60%, 70%, preferably at least 75%, 80%, 85%, more preferably at least 90%, 95%, 96%, 97%, 98%, or 99% or more of the total number of genes in the panel. In some embodiments the panel of genes selected from BCRGs, TCRGs, HLAGs, OCPGs, and CCGs comprises the genes in Table 1, 2, 3, 5, 6, 7, 8, 9, 10, 11, 12, 13, 14, 15, 16, 17, 18, 19, 20, 21, 22, or 23 or Panel A, B, C, D, E, F, G, H, I, J, K, L, M, or N. In some embodiments the panel comprises at least 2, 3, 4, 5, 6, 7, 8, 9, 10, 11, 12, 13, 14, 15, 20, 25, 30, or more of the genes in Table 1, 2, 3, 5, 6, 7, 8, 9, 10, 11, 12, 13, 14, 15, 16, 17, 18, 19, 20, 21, 22, or 23 or Panel A, B, C, D, E, F, or G, H, I J, K, L, M, or N. In some embodiments the disclosure provides a method of determining the prognosis in a breast cancer patient comprising determining the status of the genes selected from BCRGs, TCRGs, HLAGs, OCPGs, and CCGs in any one of Table 1, 2, 3, 5, 6, 7, 8, 9, 10, 11, 12, 13, 14, 15, 16, 17, 18, 19, 20, 21, 22, or 23 or Panel A, B, C, D, E, F, G, H, I, J, K, L, M or N determining the status of the ABCC5 gene or the PGR gene or both, and using the combined expression to determine the prognosis of the breast cancer.

Several panels of CCGs (shown in Tables 7, 10, 11, 12, 13, 14, 15, 16, 17, 18, 19, 20, 21, 22, or 23 and Panels A, B, C, D, E, F, G, H, I, J, L, M & N for use in combination genes selected from BCRGs, TCRGs, HLAGs, and OCPGs are useful in this regard.

TABLE 15
“Panel C”

| | Entrez | | Entrez | | Entrez |
|---|---|---|---|---|---|
| Gene Symbol | GeneID | Gene Symbol | GeneID | Gene Symbol | GeneID |
| AURKA | 6790 | DTL* | 51514 | PTTG1* | 9232 |
| BUB1* | 699 | FOXM1* | 2305 | RRM2* | 6241 |
| CCNB1* | 891 | HMMR* | 3161 | TIMELESS* | 8914 |
| CCNB2* | 9133 | KIF23* | 9493 | TPX2* | 22974 |
| CDC2* | 983 | KPNA2 | 3838 | TRIP13* | 9319 |
| CDC20* | 991 | MAD2L1* | 4085 | TTK* | 7272 |
| CDC45L* | 8318 | MELK | 9833 | UBE2C | 11065 |
| CDCA8* | 55143 | MYBL2* | 4605 | UBE2S* | 27338 |
| CENPA | 1058 | NUSAP1* | 51203 | ZWINT* | 11130 |
| CKS2* | 1164 | PBK* | 55872 | | |
| DLG7* | 9787 | PRC1* | 9055 | | |

*These genes can be used as a 26-gene subset panel (“Panel D”) in some embodiments of the disclosure.

TABLE 16
“Panel E”

| Name | GeneID | Name | GeneID | Name | GeneID |
|---|---|---|---|---|---|
| ASF1B* | 55723 | CENPM* | 79019 | ORC6L* | 23594 |
| ASPM* | 259266 | CEP55* | 55165 | PBK* | 55872 |
| BIRC5* | 332 | DLGAP5* | 9787 | PLK1* | 5347 |
| BUB1B* | 701 | DTL* | 51514 | PRC1* | 9055 |
| C18orf24* | 220134 | FOXM1* | 2305 | PTTG1* | 9232 |
| CDC2* | 983 | KIAA0101* | 9768 | RAD51* | 5888 |
| CDC20* | 991 | KIF11* | 3832 | RAD54L* | 8438 |
| CDCA3* | 83461 | KIF20A* | 10112 | RRM2* | 6241 |
| CDCA8* | 55143 | KIF4A | 24137 | TK1* | 7083 |
| CDKN3* | 1033 | MCM10* | 55388 | TOP2A* | 7153 |
| CENPF* | 1063 | NUSAP1* | 51203 | | |

*These genes can be used as a 31-gene subset panel (“Panel F”) in some embodiments of the disclosure.

TABLE 17
“Panel H”

| | | | |
|---|---|---|---|
| ASF1B* | Hs00216780_m1 | RRM2* | Hs00357247_g1 |
| ASPM* | Hs00411505_ml | TK1* | Hs01062125_ml |
| BUB1B* | Hs01084828_ml | TOP2A* | Hs00172214_ml |
| C18orf24* | Hs00536843_m1 | GAPDH | Hs99999905_m1 |
| CDC2* | Hs00364293_m1 | CLTC** | Hs00191535_m1 |
| CDKN3* | Hs00193192_m1 | MMADHC** | Hs00739517_g1 |
| CENPF* | Hs00193201_m1 | PPP2CA** | Hs00427259_m1 |
| CENPM* | Hs00608780_m1 | PSMA1** | Hs00267631_m1 |
| DTL* | Hs00978565_m1 | PSMC1** | Hs02386942_g1 |
| CDCA3* | Hs00229905_m1 | RPL13A** | Hs03043885_g1 |
| KIAA0101* | Hs00207134_m1 | RPL37** | Hs02340038_g1 |
| KIF11* | Hs00189698_m1 | RPL38** | Hs00605263_g1 |
| KIF20A* | Hs00993573_m1 | RPL4** | Hs03044647_g1 |
| KIF4A* | Hs01020169_m1 | RPL8** | Hs00361285_g1 |
| MCM10* | Hs00960349_m1 | RP529** | Hs03004310_g1 |
| NUSAP1* | Hs01006195_m1 | SLC25A3** | Hs00358082_m1 |
| PBK* | Hs00218544_m1 | TXNL1** | Hs00355488_m1 |
| PLK1* | Hs00153444_m1 | UBA52** | Hs03004332_g1 |
| PRC1* | Hs00187740_m1 | ESR1 | Hs01046815_m1; |
| | | | Hs00174860_m1 |
| PTTG1* | Hs00851754_u1 | ABCC5 | Hs00981085_m1 |
| RAD51* | Hs00153418_m1 | PGR | Hs00172183_m1 |
| RAD54L* | Hs00269177_m1 | | |

*CCP genes (i.e., Panel I)
# CCP genes plus ESR1, ABCC5, and PGR (Panel J).
¹ Note
that in some embodiments utilizing Panel J, ESR1 is optional and is analyzed primarily as a confirmation of the tumor's ER+ status. Thus, in some embodiments Panel J lacks ESR1.
**Housekeeping genes (Panel K)

TABLE 18
“Panel L”

| | | Entrez | | | Entrez |
|---|---|---|---|---|---|
| Gene Symbol | ABI Assay ID | GeneID | Gene Symbol | ABI Assay ID | GeneID |
| ASF1B*# | Hs00216780_m1 | 55723 | RRM2*# | Hs00357247_g1 | 6241 |
| ASPM*# | Hs00411505_m1 | 259266 | TK1*# | Hs01062125_m1 | 7083 |
| BUB1B*# | Hs01084828_m1 | 701 | TOP2A*# | Hs00172214_m1 | 7153 |
| C18orf24*# | Hs00536843_m1 | 220134 | GAPDH\hat{ } | Hs99999905_m1 | 2597 |
| CDC2*# | Hs00364293_m1 | 983 | CLTC** | Hs00191535_m1 | 1213 |
| CDKN3*# | Hs00193192_m1 | 83461 | MMADHC** | Hs00739517_g1 | 27249 |
| CENPF*# | Hs00193201_m1 | 1033 | PPP2CA** | Hs00427259_m1 | 5515 |
| CENPM*# | Hs00608780_m1 | 1063 | PSMA1** | Hs00267631_m1 | 5682 |
| DTL*# | Hs00978565_m1 | 79019 | PSMC1** | Hs02386942_g1 | 5700 |
| CDCA3*# | Hs00229905_m1 | 51514 | RPL13A** | Hs03043885_g1 | 23521 |
| KIAA0101*# | Hs00207134_m1 | 9768 | RPL37** | Hs02340038_g1 | 6167 |
| KIF11*# | Hs00189698_m1 | 3832 | RPL38** | Hs00605263_g1 | 6169 |
| KIF20A*# | Hs00993573_m1 | 10112 | RPL4** | Hs03044647_g1 | 6124 |
| MCM10*# | Hs00960349_m1 | 55388 | RPL8** | Hs00361285_g1 | 6132 |
| NUSAP1*# | Hs01006195_m1 | 51203 | RP529** | Hs03004310_g1 | 6235 |
| PBK*# | Hs00218544_m1 | 55872 | SLC25A3** | Hs00358082_m1 | 6515 |
| PLK1*# | Hs00153444_m1 | 5347 | TXNL1** | Hs00355488_m1 | 9352 |
| PRC1*# | Hs00187740_m1 | 9055 | UBA52** | Hs03004332_g1 | 7311 |
| PTTG1*# | Hs00851754_u1 | 9232 | ESR1#¹ | Hs01046815_m1 | 2099 |
| | | | | Hs00174860_m1 | |
| RAD51*# | Hs00153418_m1 | 5888 | ABCC5# | Hs00981085_m1 | 10057 |
| RAD54L*# | Hs00269177_m1 | 8438 | PGR# | Hs00172183_m1 | 5241 |

*CCP genes (Panel M)
#CCP genes plus ESR1, ABCC5, and PGR (Panel N).
¹Note
that in some embodiments utilizing Panel N, ESR1 is optional and is analyzed primarily as a confirmation of the tumor's ER+ status. Thus, in some embodiments Panel J lacks ESR1.
**Housekeeping genes
\hat{ }Internal control gene

Similar to Tables 7 and 10 to 14 above, the CCP genes in Tables 17 & 18 were ranked according to correlation to the CCP mean and according to independent predictive value (p-value). Rankings according to correlation to the mean are shown in Tables 19 to 21 below. Rankings according to p-value are shown in Tables 22 & 23 below.

TABLE 19

| Gene # | Gene Symbol |
|---|---|
| 1 | KIF4A |
| 2 | CDC2 |
| 3 | PRC1 |
| 4 | TOP2A |
| 5 | KIF20A |
| 6 | BUB1B |
| 7 | CDKN3 |
| 8 | PTTG1 |
| 9 | NUSAP1 |
| 10 | KIF11 |
| 11 | ASPM |
| 12 | RRM2 |
| 13 | CENPF |
| 14 | KIAA0101 |
| 15 | PBK |
| 16 | MCM10 |
| 17 | RAD51 |
| 18 | CDCA3 |
| 19 | ASF1B |
| 20 | DTL |
| 21 | PLK1 |
| 22 | CENPM |
| 23 | TK1 |
| 24 | C18orf24 |
| 25 | RAD54L |

TABLE 20

| Gene # | Gene Symbol |
|---|---|
| 1 | CDKN3 |
| 2 | CDC2 |
| 3 | KIF11 |
| 4 | KIAA0101 |
| 5 | NUSAP1 |
| 6 | CENPF |
| 7 | ASPM |
| 8 | BUB1B |
| 9 | RRM2 |
| 10 | KIF20A |
| 11 | PLK1 |
| 12 | TOP2A |
| 13 | TK1 |
| 14 | PBK |
| 15 | ASF1B |
| 16 | C18orf24 |
| 17 | RAD54L |
| 18 | PTTG1 |
| 19 | KIF4A |
| 20 | CDCA3 |
| 21 | MCM10 |
| 22 | PRC1 |
| 23 | DTL |
| 24 | RAD51 |
| 25 | CENPM |

TABLE 21

| Gene # | Gene Symbol |
|---|---|
| 1 | ASPM |
| 2 | KIF11 |
| 3 | MCM10 |
| 4 | PRC1 |
| 5 | BUB1B |
| 6 | NUSAP1 |
| 7 | C18orf24 |
| 8 | PLK1 |
| 9 | CDKN3 |
| 10 | RRM2 |
| 11 | RAD51 |
| 12 | RAD54L |
| 13 | CDC2 |
| 14 | CENPF |
| 15 | TOP2A |
| 16 | KIF20A |
| 17 | KIAA0101 |
| 18 | CDCA3 |
| 19 | ASF1B |
| 20 | CENPM |
| 21 | TK1 |
| 22 | PBK |
| 23 | PTTG1 |
| 24 | DTL |
| 25 | KIF4A |

TABLE 22

| Gene # | Gene Symbol |
|---|---|
| 1 | NUSAP1 |
| 2 | CDC2 |
| 3 | RRM2 |
| 4 | PTTG1 |
| 5 | PBK |
| 6 | PRC1 |
| 7 | DTL |
| 8 | ASF1B |
| 9 | ASPM |
| 10 | BUB1B |
| 11 | C18orf24 |
| 12 | CDCA3 |
| 13 | CDKN3 |
| 14 | CENPF |
| 15 | CENPM |
| 16 | KIAA0101 |
| 17 | KIF11 |
| 18 | KIF20A |
| 19 | KIF4A |
| 20 | MCM10 |
| 21 | PLK1 |
| 22 | RAD51 |
| 23 | RAD54L |
| 24 | TK1 |
| 25 | TOP2A |

TABLE 23

| Gene # | Gene Symbol |
|---|---|
| 1 | MCM10 |
| 2 | ASPM |
| 3 | CENPF |
| 4 | TOP2A |
| 5 | NUSAP1 |
| 6 | CDKN3 |
| 7 | KIF11 |
| 8 | KIF20A |
| 9 | BUB1B |
| 10 | RAD54L |
| 11 | TK1 |
| 12 | DTL |
| 13 | PRC1 |
| 14 | PTTG1 |
| 15 | CDC2 |
| 16 | PLK1 |
| 17 | C18orf24 |
| 18 | RRM2 |
| 19 | CENPM |
| 20 | RAD51 |
| 21 | KIAA0101 |
| 22 | CDCA3 |
| 23 | PBK |
| 24 | ASF1B |
| 25 | KIF4A |

The rankings of each gene according to correlation to the mean (Tables 7, 10 & 12) and p-value (Tables 13 & 14) were used to derive two different combination rankings. Table 24 ranks the CCP genes of Table 19 according to the highest unweighted combination score calculated by the following formula: Combination score for each gene=(1/(correlation in Table 7))+(1/(correlation in Table 12))+(1/(correlation in Table 14))+(1/(p-value in Table 15))+(1/(p-value in Table 16)). Table 25 ranks the CCP genes of Table 19 according to the highest weighted combination score (which gives greater weight to p-value over correlation to the mean) calculated by the following formula: Combination score for each gene=(2/(correlation in Table 7))+(3/(correlation in Table 12))+(5/(correlation in Table 14))+(7/(p-value in Table 15))+(10/(p-value in Table 16)).

TABLE 24

| Gene # | Gene Symbol |
|---|---|
| 1 | NUSAP1 |
| 2 | MCM10 |
| 3 | ASPM |
| 4 | CDC2 |
| 5 | KIF11 |
| 6 | CDKN3 |
| 7 | CENPF |
| 8 | KIF4A |
| 9 | PRC1 |
| 10 | BUB1B |
| 11 | RRM2 |
| 12 | TOP2A |
| 13 | PTTG1 |
| 14 | KIF20A |
| 15 | KIAA0101 |
| 16 | PLK1 |
| 17 | PBK |
| 18 | C18orf24 |
| 19 | RAD54L |
| 20 | DTL |
| 21 | TK1 |
| 22 | RAD51 |
| 23 | ASF1B |
| 24 | CDCA3 |
| 25 | CENPM |

TABLE 25

| Gene # | Gene Symbol |
|---|---|
| 1 | NUSAP1 |
| 2 | CDC2 |
| 3 | KIF11 |
| 4 | ASPM |
| 5 | CDKN3 |
| 6 | BUB1B |
| 7 | PRC1 |
| 8 | RRM2 |
| 9 | CENPF |
| 10 | TOP2A |
| 11 | KIF20A |
| 12 | PTTG1 |
| 13 | MCM10 |
| 14 | KIAA0101 |
| 15 | PBK |
| 16 | PLK1 |
| 17 | DTL |
| 18 | KIF4A |
| 19 | RAD51 |
| 20 | C18orf24 |
| 21 | ASF1B |
| 22 | CDCA3 |
| 23 | TK1 |
| 24 | RAD54L |
| 25 | CENPM |

In the expression signatures the particular genes selected from BCRGs, TCRGs, HLAGs, OCPGs, and/or CCGs assayed is often not as important as the total number of genes. The number of genes selected from BCRGs, TCRGs, HLAGs, OCPGs, and/or CCGs that are assayed can vary depending on many factors, e.g., technical constraints, cost considerations, the classification being made, the cancer being tested, the desired level of predictive power, etc. Increasing the number of genes selected from BCRGs, TCRGs, HLAGs, OCPGs, and/or CCGs that are assayed in a panel according to the disclosure is, as a general matter, advantageous because, e.g., a larger pool of mRNAs to be assayed means less “noise” caused by outliers and less chance of an assay error throwing off the overall predictive power of the test. However, cost and other considerations will generally limit this number and finding the optimal number of genes selected from BCRGs, TCRGs, HLAGs, OCPGs, and/or CCGs for a signature is desirable.

It has been discovered that the predictive power of a CCG (and analogously genes from BCRGs, TCRGs, HLAGs, OCPGs, and CCGs) signature often ceases to increase significantly beyond a certain number of genes. By way of example, in order to determine the optimal number of cell cycle genes for the signature, the predictive power of the mean was tested for randomly selected sets of from 1 to 30 of the CCGs in Panel C. This demonstrates, for some embodiments of the disclosure, a threshold number of CCGs in a panel (10, 15, or between 10 and 15) that provides significantly improved predictive power. In some embodiments even smaller panels of CCGs are sufficient to prognose disease outcome and/or predict therapy response/benefit. To evaluate how even smaller subsets of a larger CCG set (i.e., smaller CCG subpanels) performed, the inventors compared how well the CCGs from Panel C predicted outcome as a function of the number of CCGs included in the signature. As shown in Table 26 below, small CCG signatures (e.g., 2, 3, 4, 5, 6 CCGs, etc.) are significant predictors and analogously small signatures of genes selected from BCRGs, TCRGs, HLAGs, and OCPGs, alone, or in combination with CCGs.

TABLE 26

| # of CCGs | Mean of log10 (p-value)* |
|---|---|
| 1 | −3.579 |
| 2 | −4.279 |
| 3 | −5.049 |
| 4 | −5.473 |
| 5 | −5.877 |
| 6 | −6.228 |

*For 1000 randomly drawn subsets, size 1 through 6, of CCGs.

In some embodiments, the optimal number of CCGs in a signature (n_{O}) can be found wherever the following is true

(Pₙ₊₁−Pₙ)<C_{O},

wherein P is the predictive power (i.e., Pₙ is the predictive power of a signature with n genes and Pₙ₊₁ is the predictive power of a signature with n genes plus one) and C_{O} is some optimization constant. Predictive power can be defined in many ways known to those skilled in the art including, but not limited to, the signature's p-value. C_{O} can be chosen by the artisan based on his or her specific constraints. For example, if cost is not a critical factor and extremely high levels of sensitivity and specificity are desired, C_{O} can be set very low such that only trivial increases in predictive power are disregarded. On the other hand, if cost is decisive and moderate levels of sensitivity and specificity are acceptable, C_{O} can be set higher such that only significant increases in predictive power warrant increasing the number of genes in the signature. The same priniciples also hold true on a general level when considering panels of genes selected from BCRGs, TCRGs, HLAGs, OCPGs, alone, or in combination with CCGs.

Alternatively, a graph of predictive power as a function of gene number may be plotted and the second derivative of this plot taken. The point at which the second derivative decreases to some predetermined value (C_{O}′) may be the optimal number of genes in the signature. It has been shown that p-values ceased to improve significantly between about 10 and about 15 genes (e.g., CCGs, or analogously genes selected from BCRGs, TCRGs, HLAGs, OCPGs, and CCGs), thus indicating that an optimal number of genes (e.g., CCGs, or analogously genes selected from BCRGs, TCRGs, HLAGs, OCPGs, and CCGs) in a prognostic panel is from about 10 to about 15. Thus, in some preferred embodiments of the disclosure, between about 10 and about 15 genes (e.g., CCGs, or analogously genes selected from BCRGs, TCRGs, HLAGs, OCPGs, and CCGs) are used in addition to the ABCC5 gene or the PGR gene or both. In some embodiments the panel comprises between about 10 and about 15 genes (e.g., CCGs, or analogously genes selected from BCRGs, TCRGs, HLAGs, OCPGs, and CCGs) and the genes constitute at least 80% of the panel (or are weighted to contribute at least 75%). In other embodiments the panel comprises CCGs plus one or more additional markers selected from BCRGs, TCRGs, HLAGs, and OCPGs, that significantly increase the predictive power of the panel (i.e., make the predictive power significantly better than if the panel consisted of only the CCGs). Any other combination of CCGs (including any of those listed in Table 7, 8, 9, 10, 11, 12, 13, or 14 or Panel A, B, C, D, E, F, or G) in combination with at least 2, 4, 6, 8, 10, or 12 or more genes selected from BCRGs, TCRGs, HLAGs, and OCPGs (including any of those listed in Table 1, 2, 3, 4, 5, or 6), can be used to practice the disclosure.

In some embodiments the panel comprises at least 3, 4, 5, 6, 7, 8, 9, 10, 15, 20, 25, 30, 35, 40, 45, 50 or more CCGs, in combination with at least 2, 4, 6, 8, 10, or 12 or more genes selected from BCRGs, TCRGs, HLAGs, and OCPGs. In some embodiments the panel comprises between 5 and 100 CCGs in combination with at least 2, 4, 6, 8, 10, or 12 or more genes selected from BCRGs, TCRGs, HLAGs, and OCPGs, between 7 and 40 CCGs in combination with at least 2, 4, 6, 8, 10, or 12 or more genes selected from BCRGs, TCRGs, HLAGs, and OCPGs, between 5 and 25 CCGs in combination with at least 2, 4, 6, 8, 10, or 12 or more genes selected from BCRGs, TCRGs, HLAGs, and OCPGs, between 10 and 20 CCGs in combination with at least 2, 4, 6, 8, 10, or 12 or more genes selected from BCRGs, TCRGs, HLAGs, and OCPGs, or between 10 and 15 CCGs in combination with at least 2, 4, 6, 8, 10, or 12 or more genes selected from BCRGs, TCRGs, HLAGs, and OCPGs. In some embodiments CCGs, BCRGs, TCRGs, HLAGs and OCPGs comprise at least a certain proportion of the panel. Thus, in some embodiments, the panel comprises at least 25%, 30%, 40%, 50%, 60%, 70%, 75%, 80%, 85%, 90%, 95%, 96%, 97%, 98%, or 99% genes selected from CCGs, BCRGs, TCRGs, HLAGs and OCPGs. In some embodiments, the CCGs are any of the genes listed in Table 7, 8, 9, 10, 11, 12, 13, or 14 or Panel A, B, C, D, E, F, or G, in combination with at least 2, 4, 6, 8, 10, or 12 or more genes selected from BCRGs, TCRGs, HLAGs, and OCPGs are any of those listed in Table 1, 2, 3, 4, 5, or 6. In some embodiments the panel comprises at least 2, 3, 4, 5, 6, 7, 8, 9, 10, 15, 20, 25, 30, 35, 40, 45, 50 or more genes in any of Table 7, 8, 9, 10, 11, 12, 13, or 14 or Panel A, B, C, D, E, F, or G in combination with at least 2, 4, 6, 8, 10, or 12 or more genes selected from BCRGs, TCRGs, HLAGs, and OCPGs as in any of Table 1, 2, 3, 4, 5, or 6. In some embodiments the panel comprises all of the genes in any of Table 7, 8, 9, 10, 11, 12, 13, or 14 or Panel A, B, C, D, E, F, or G, in combination with at least 2, 4, 6, 8, 10, or 12 or more genes selected from BCRGs, TCRGs, HLAGs, and OCPGs as in any of Table 1, 2, 3, 4, 5, or 6.

As mentioned above, many of the BCRGs, TCRGs, HLAGs, OCPGs, and CCGs of the disclosure have been analyzed to determine their correlation to the their respective mean and also, to determine their relative predictive value within a panel (see Tables 1, 2, 3, 4, 5, 6, 7, 8, 9, 10, 11, 12, 13, 14, 19, 20, 21, 22, and 23 and Panels A, B, C, D, E, F, G, and H). Thus, in some embodiments the plurality of test genes comprises at least some number of BCRGs, TCRGs, HLAGs, OCPGs, and CCGs (e.g., at least 3, 4, 5, 6, 7, 8, 9, 10, 15, 20, 25, 30, 35, 40, 45, 50 or more BCRGs, TCRGs, HLAGs, OCPGs, and CCGs) and this plurality of BCRGs, TCRGs, HLAGs, OCPGs, and CCGs comprises the top 2, 3, 4, 5, 6, 7, 8, 9, 10, 11, 12, 13, 14, 15, 20, 25, 30, 35, 40 or more BCRGs, TCRGs, HLAGs, OCPGs, and CCGs listed in any of Tables 1, 6A, 6B, 8, 9, 30, 31, 32, or 33, and/or any of Tables 10, 11, 12, 13, 14, 15, 19, 20, 21, 22, 23, 24, 25, 34, and 35. In some embodiments, the plurality of test genes comprises at least some number of BCRGs, TCRGs, HLAGs, OCPGs, and CCGs (e.g., at least 3, 4, 5, 6, 7, 8, 9, 10, 15, 20, 25, 30, 35, 40, 45, 50 or more BCRGs, TCRGs, HLAGs, OCPGs, and CCGs) and this plurality of CCGs comprises at least 1, 2, 3, 4, 5, 6, 7, 8, 9, 10, 15, or 20 of the following genes: ASPM, BIRC5, BUB1B, CCNB2, CDC2, CDC20, CDCAB, CDKN3, CENPF, DLGAP5, FOXM1, KIAA0101, KIF11, KIF2C, KIF4A, MCM10, NUSAP1, PRC1, RACGAP1, and TPX2. In some embodiments the plurality of test genes comprises at least some number of BCRGs, TCRGs, HLAGs, OCPGs, and CCGs (e.g., at least 3, 4, 5, 6, 7, 8, 9, 10, 15, 20, 25, 30, 35, 40, 45, 50 or more BCRGs, TCRGs, HLAGs, OCPGs, and CCGs) and this plurality of BCRGs, TCRGs, HLAGs, OCPGs, and CCGs comprises any one, two, three, four, five, six, seven, eight, nine, or ten or all of gene numbers 1 & 2, 1 to 3, 1 to 4, 1 to 5, 1 to 6, 1 to 7, 1 to 8, 1 to 9, or 1 to 10 of any of Tables 1, 6A, 6B, 8, 9, 30, 31, 32, or 33, and/or any of Tables 10, 11, 12, 13, 14, 15, 19, 20, 21, 22, 23, 24, 25, 34, and 35. In some embodiments the plurality of test genes comprises at least some number of BCRGs, TCRGs, HLAGs, OCPGs, and CCGs (e.g., at least 3, 4, 5, 6, 7, 8, 9, 10, 15, 20, 25, 30, 35, 40, 45, 50 or more BCRGs, TCRGs, HLAGs, OCPGs, and CCGs) and this plurality of BCRGs, TCRGs, HLAGs, OCPGs, and CCGs comprises any one, two, three, four, five, six, seven, eight, or nine or all of gene numbers 2 & 3, 2 to 4, 2 to 5, 2 to 6, 2 to 7, 2 to 8, 2 to 9, or 2 to 10 of any of Tables 1, 6A, 6B, 8, 9, 30, 31, 32, or 33, and/or any of Tables 10, 11, 12, 13, 14, 15, 19, 20, 21, 22, 23, 24, 25, 34, and 35. In some embodiments the plurality of test genes comprises at least some number of BCRGs, TCRGs, HLAGs, OCPGs, and CCGs (e.g., at least 3, 4, 5, 6, 7, 8, 9, 10, 15, 20, 25, 30, 35, 40, 45, 50 or more BCRGs, TCRGs, HLAGs, OCPGs, and CCGs) and this plurality of BCRGs, TCRGs, HLAGs, OCPGs, and CCGs comprises any one, two, three, four, five, six, seven, or eight or all of gene numbers 3 & 4, 3 to 5, 3 to 6, 3 to 7, 3 to 8, 3 to 9, or 3 to 10 of any of Tables 1, 6A, 6B, 8, 9, 30, 31, 32, or 33, and/or any of Tables 10, 11, 12, 13, 14, 15, 19, 20, 21, 22, 23, 24, 25, 34, and 35. In some embodiments the plurality of test genes comprises at least some number of BCRGs, TCRGs, HLAGs, OCPGs, and CCGs (e.g., at least 3, 4, 5, 6, 7, 8, 9, 10, 15, 20, 25, 30, 35, 40, 45, 50 or more BCRGs, TCRGs, HLAGs, OCPGs, and CCGs) and this plurality of BCRGs, TCRGs, HLAGs, OCPGs, and CCGs comprises any one, two, three, four, five, six, or seven or all of gene numbers 4 & 5, 4 to 6, 4 to 7, 4 to 8, 4 to 9, or 4 to 10 of any of Tables 1, 6A, 6B, 8, 9, 30, 31, 32, or 33, and/or any of Tables 10, 11, 12, 13, 14, 15, 19, 20, 21, 22, 23, 24, 25, 34, and 35. In some embodiments the plurality of test genes comprises at least some number of BCRGs, TCRGs, HLAGs, OCPGs, and CCGs (e.g., at least 3, 4, 5, 6, 7, 8, 9, 10, 15, 20, 25, 30, 35, 40, 45, 50 or more BCRGs, TCRGs, HLAGs, OCPGs, and CCGs) and this plurality of BCRGs, TCRGs, HLAGs, OCPGs, and CCGs comprises any one, two, three, four, five, six, seven, eight, nine, 10, 11, 12, 13, 14, or 15 or all of gene numbers 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Tables 1, 6A, 6B, 8, 9, 30, 31, 32, or 33, and/or any of Tables 10, 11, 12, 13, 14, 15, 19, 20, 21, 22, 23, 24, 25, 34, and 35.

In some such embodiments, multiple scores (e.g., ISG, OCPG, CCG, ABCC5, clinical parameters or scores) can be combined into a more comprehensive score. Single component (e.g., ISG) or combined test scores for a particular patient can be compared to single component or combined scores for reference populations as described herein, with differences between test and reference scores being correlated to or indicative of some clinical feature. Thus, in some embodiments the disclosure provides a method of determining a cancer patient's prognosis (or some other clinical feature as described herein) comprising (1) obtaining the measured expression levels of a plurality of gene comprising a plurality of ISGs and/or OCPGs (as described throughout this document) in a sample from the patient, (2) calculating a test value from these measured expression levels, (3) comparing said test value to a reference value calculated from measured expression levels of the plurality of genes in a reference population of patients, and (4)(a) correlating a test value greater than the reference value to a poor prognosis (or other unfavorable clinical feature as described herein) or (4)(b) correlating a test value equal to or less than the reference value to a good prognosis (or other favorable clinical feature as described herein).

In some such embodiments the test value is calculated by averaging the measured expression of the plurality of genes (as discussed below). In some embodiments the test value is calculated by weighting each of the plurality of genes in a particular way.

In some embodiments the plurality of CCGs are weighted such that they contribute at least some proportion of the test value (e.g., 10%, 20%, 30%, 40%, 50%, 60%, 70%, 80%, 90%, 95%, 99%, 100%). In some embodiments each of the plurality of genes is weighted such that not all are given equal weight (e.g., a particular ISG, OCPG or CCG weighted to contribute more to the test value than one, some or all other ISGs, OCPGs or CCGs in the plurality).

In some embodiments the disclosure provides an method of determining a cancer patient's prognosis (or some other clinical feature as described herein) comprising: (1) obtaining the measured expression levels of a plurality of gene comprising a plurality of ISGs and/or OCPGs (as described throughout this document) in a sample from the patient; (2) obtaining one or more scores for the patient comprising (or calculated or derived from or reflecting) one or more clinical features (e.g., age, grade, tumor size, node status (including number of positive nodes, if any), hormone therapy); (3) deriving a combined test value from the measured levels obtained in (1) and the score(s) obtained in (2); (4) comparing the combined test value to a combined reference value derived from measured expression levels of the plurality of genes and a score comprising one or more clinical features in a reference population of patients; and (5)(a) correlating a combined test value greater than the combined reference value to a poor prognosis (or some other unfavorable clinical feature as described herein) or (5)(b) correlating a combined test value equal to or less than the combined reference value to a good prognosis (or some other favorable clinical feature as described herein).

In some embodiments the combined score includes molecular markers such as any combination of ISG/OCPG (for convenience in these embodiments termed “Immune gene expression,” with the score for the total expression of a panel of these genes being term the “Immune score”), CCP gene expression (CCP score), ABCC5 expression, and PGR expression. Immune gene expression, CCP gene expression, and ABCC5 expression can be continuous numeric variables. In some embodiments described herein, e.g., Examples 6 & 7, such combined scores are called molecular scores. Such combined scores can be used as test values (or correspondingly reference values) in any embodiments (e.g., methods or systems) of the disclosure. In some embodiments such a combined score is calculated according to the following formula:

Combined Score=(A×CCP score)−(B×Immune score)+(C×ABCC5)−(D×PGR). (1)

In some embodiments A=0.436, B=0.189, C=0.155, and D=0.086. In some embodiments A=0.0436 to 0.8284, 0.0872 to 0.7848, 0.1308 to 0.7412, 0.1744 to 0.6976, 0.218 to 0.654, 0.2616 to 0.6104, 0.3052 to 0.5668, 0.3488 to 0.5232, 0.3924 to 0.4796, or any single value between any of these ranges out to four decimal places. In some embodiments A=0.0436 to 4.36, 0.0872 to 3.924, 0.1308 to 3.488, 0.1744 to 3.052, 0.218 to 2.616, 0.2616 to 2.18, 0.3052 to 1.744, 0.3488 to 1.308, 0.3924 to 0.872, or any single value between any of these ranges out to four decimal places. In some embodiments B=0.0189 to 0.3591, 0.0378 to 0.3402, 0.0567 to 0.3213, 0.0756 to 0.3024, 0.0945 to 0.2835, 0.1134 to 0.2646, 0.1323 to 0.2457, 0.1512 to 0.2268, 0.1701 to 0.2079, or any single value between any of these ranges out to four decimal places. In some embodiments B=0.0189 to 1.89, 0.0378 to 1.701, 0.0567 to 1.512, 0.0756 to 1.323, 0.0945 to 1.134, 0.1134 to 0.945, 0.1323 to 0.756, 0.1512 to 0.567, 0.1701 to 0.378, or any single value between any of these ranges out to four decimal places. In some embodiments C=0.0155 to 0.2945, 0.031 to 0.279, 0.0465 to 0.2635, 0.062 to 0.248, 0.0775 to 0.2325, 0.093 to 0.217, 0.1085 to 0.2015, 0.124 to 0.186, 0.1395 to 0.1705, or any single value between any of these ranges out to four decimal places. In some embodiments C=0.0155 to 1.55, 0.031 to 1.395, 0.0465 to 1.24, 0.062 to 1.085, 0.0775 to 0.93, 0.093 to 0.775, 0.1085 to 0.62, 0.124 to 0.465, 0.1395 to 0.31, or any single value between any of these ranges out to four decimal places. In some embodiments D=0.0086 to 0.1634, 0.0172 to 0.1548, 0.0258 to 0.1462, 0.0344 to 0.1376, 0.043 to 0.129, 0.0516 to 0.1204, 0.0602 to 0.1118, 0.0688 to 0.1032, 0.0774 to 0.0946, or any single value between any of these ranges out to four decimal places. In some embodiments D=0.0086 to 0.86, 0.0172 to 0.774, 0.0258 to 0.688, 0.0344 to 0.602, 0.043 to 0.516, 0.0516 to 0.43, 0.0602 to 0.344, 0.0688 to 0.258, 0.0774 to 0.172, or any single value between any of these ranges out to four decimal places.

In some embodiments the combined score includes a molecular score as described above combined with clinical parameters, e.g., any combination of tumor size, tumor grade and/or node status. In some embodiments described herein, e.g., Examples 6 & 7, such combined scores are called molecular scores. Tumor size can be a continuous numeric variable with, e.g., size being expressed in centimeters. Tumor grade can be a continuous numeric variable (e.g., the integer number of the grade, e.g., grade 1, 2, or 3). Node status can be a continuous numeric variable (e.g., the integer number of positive nodes). Alternatively a specific value can be incorporated (e.g., added) into the combined score for any particular grade or node status. Such combined scores can be used as test values (or correspondingly reference values) in any embodiments (e.g., methods or systems) of the disclosure. In some embodiments such a combined score is calculated according to any of the following formulae:

Combined score=(Molecular score as described above)+(A×Tumor size (cm))+(either B (if Grade 2) or C (if Grade 3))+(D (if N1)). (2)

In some embodiments A=0.202, B=0.378, C=0.777, and D=0.589. In some embodiments A=0.0202 to 0.3838, 0.0404 to 0.3636, 0.0606 to 0.3434, 0.0808 to 0.3232, 0.101 to 0.303, 0.1212 to 0.2828, 0.1414 to 0.2626, 0.1616 to 0.2424, 0.1818 to 0.2222, or any single value between any of these ranges out to four decimal places. In some embodiments A=0.0202 to 2.02, 0.0404 to 1.818, 0.0606 to 1.616, 0.0808 to 1.414, 0.101 to 1.212, 0.1212 to 1.01, 0.1414 to 0.808, 0.1616 to 0.606, 0.1818 to 0.404, or any single value between any of these ranges out to four decimal places. In some embodiments B=0.0378 to 0.7182, 0.0756 to 0.6804, 0.1134 to 0.6426, 0.1512 to 0.6048, 0.189 to 0.567, 0.2268 to 0.5292, 0.2646 to 0.4914, 0.3024 to 0.4536, 0.3402 to 0.4158, or any single value between any of these ranges out to four decimal places. In some embodiments B=0.0378 to 3.78, 0.0756 to 3.402, 0.1134 to 3.024, 0.1512 to 2.646, 0.189 to 2.268, 0.2268 to 1.89, 0.2646 to 1.512, 0.3024 to 1.134, 0.3402 to 0.756, or any single value between any of these ranges out to four decimal places. In some embodiments C=0.0777 to 1.4763, 0.1554 to 1.3986, 0.2331 to 1.3209, 0.3108 to 1.2432, 0.3885 to 1.1655, 0.4662 to 1.0878, 0.5439 to 1.0101, 0.6216 to 0.9324, 0.6993 to 0.8547, or any single value between any of these ranges out to four decimal places. In some embodiments C=0.0777 to 7.77, 0.1554 to 6.993, 0.2331 to 6.216, 0.3108 to 5.439, 0.3885 to 4.662, 0.4662 to 3.885, 0.5439 to 3.108, 0.6216 to 2.331, 0.6993 to 1.554, or any single value between any of these ranges out to four decimal places. In some embodiments D=0.0589 to 1.1191, 0.1178 to 1.0602, 0.1767 to 1.0013, 0.2356 to 0.9424, 0.2945 to 0.8835, 0.3534 to 0.8246, 0.4123 to 0.7657, 0.4712 to 0.7068, 0.5301 to 0.6479, or any single value between any of these ranges out to four decimal places. In some embodiments D=0.0589 to 5.89, 0.1178 to 5.301, 0.1767 to 4.712, 0.2356 to 4.123, 0.2945 to 3.534, 0.3534 to 2.945, 0.4123 to 2.356, 0.4712 to 1.767, 0.5301 to 1.178, or any single value between any of these ranges out to four decimal places.

In some embodiments the combined score includes any combination of Immune gene expression (Immune score as discussed above), CCP gene expression (CCP score as discussed above), ABCC5 expression, PGR expression, tumor size, tumor grade, and/or node status (e.g., number of positive nodes). Immune gene expression, CCP gene expression, ABCC5 expression and/or PGR expression can be continuous numeric variables. Tumor size can be a continuous numeric variable with, e.g., size being expressed in centimeters. Tumor grade can be a continuous numeric variable (e.g., the integer number of the grade, e.g., grade 1, 2, or 3). Node status can be a continuous numeric variable (e.g., the integer number of positive nodes). Such combined scores can be used as test values (or correspondingly reference values) in any methods or systems of the disclosure.

In some embodiments the combined score is calculated according to any of the following formulae:

Combined score=(D×Tumor Size (cm))+(E×# of positive Nodes)+(B×CCP score)−(A×Immune score)+(C×ABCC5) (3)

Combined score=(D×Tumor Size (cm))+(E×node status [0 or 1])+(B×CCP score)−(A×Immune score)+(C×ABCC5)−(F×PGR) (4)

In some embodiments one or more of the clinical variables (e.g., tumor size and node status) can be combined into a clinical score (e.g., nomogram score), which can then be combined with one or more of the gene expression scores score to yield a combined score according to the following more generalized formula:

Combined score=A*(expression score)+B*(clinical score) (5)

In some embodiments, any of formulae (1), (2), (3), (4) and/or (5) are used in the methods, systems, etc. of the disclosure to determine prognosis based on a patient's sample. In some embodiments, Immune score and/or CCP score are the unweighted mean of C_{T} values for the expression of genes in each group (e.g., immune mean expression of immune genes, mean of CCP genes, etc.) being analyzed, optionally normalized by the unweighted mean of the HK genes so that higher values indicate higher expression (in some embodiments one unit is equivalent to a two-fold change in expression).

In some embodiments A=0.45, B=0.52, C=0.50, D=0.60, and E=0.64. In some embodiments A=0.44, B=0.54, C=0.40, D=0.48, E=0.73, and F=0.09. In some embodiments, A, B, C, D, and/or E is within rounding of these values (e.g., A is between 0.445 and 0.454, etc.). In some cases a formula may not have all of the specified coefficients or have the value of 0 for one or more of the coefficients (and thus not incorporate the corresponding variable(s)). For example, one of the embodiments mentioned previously may incorporate formula (1) where A in formula (1) is 0.95 and B in formula (2) is 0.61. C, D and E would not be applicable in this example. In some embodiments A is between 0.4 and 0.5, 0.4 and 0.49, 0.4 and 0.45, 0.35 and 0.45, 0.36 and 0.45, 0.37 and 0.45, 0.38 and 0.45, 0.39 and 0.45, 0.35 and 0.4, 0.3 and 0.45, 0.3 and 0.4, 0.3 and 0.45, 0.25 and 0.49, 0.25 and 0.45, 0.25 and 0.4, 0.25 and 0.35, or between 0.25 and 0.3. In some embodiments B is between 0.35 and 1, 0.40 and 0.99, 0.45 and 0.95, 0.45 and 0.8, 0.45 and 0.7, 0.45 and 0.65, 0.50 and 0.63, or between 0.50 and 0.54. In some embodiments C is between 0.10 and 1, 0.15 and 0.95, 0.20 and 0.90, 0.25 and 0.8, 0.30 and 0.7, 0.35 and 0.65, 0.40 and 0.60, or between 0.45 and 0.55. In some embodiments D is between 0.20 and 1, 0.25 and 0.95, 0.30 and 0.90, 0.35 and 0.85, 0.40 and 0.80, 0.45 and 0.75, 0.50 and 0.70, or between 0.55 and 0.65. In some embodiments D is between 0.20 and 1, 0.25 and 0.75, 0.30 and 0.65, 0.35 and 0.55, 0.40 and 0.50, or between 0.45 and 0.50. In some embodiments E is between 0.20 and 1, 0.25 and 0.95, 0.30 and 0.90, 0.35 and 0.85, 0.40 and 0.80, 0.45 and 0.75, 0.50 and 0.70, or between 0.55 and 0.65. In some embodiments E is between 0.20 and 1, 0.30 and 0.95, 0.30 and 0.90, 0.40 and 0.85, 0.50 and 0.80, 0.60 and 0.75, or between 0.70 and 0.75. In some embodiments F is between 0.001 and 0.2, 0.005 and 0.18, 0.01 and 0.16, 0.02 and 0.14, 0.04 and 0.12, 0.06 and 0.11, or between 0.08 and 0.10.

In some embodiments A is between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20; B is between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20; C is between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20; and D is between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20; and E is between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20. In some embodiments, A, B, and/or C is within rounding of any of these values (e.g., A is between 0.45 and 0.54, etc.).

Many cancer patients have surgery to remove the tumor (sometimes including surrounding healthy tissue) as the standard of care or initial treatment. In one aspect, the disclosure is related to the prognosis of such patients by determining the gene expression signatures as disclosed and described herein. By way of example, for many breast cancer patients and their physicians, surgery to remove the tumor (sometimes including surrounding healthy tissue) is the standard of care. Because surgery can cure some patients and adjuvant chemotherapy is debilitating and expensive, the decision whether to undertake adjuvant chemotherapy is more difficult. For patients identified according to the methods described above as having a poor prognosis or decreased probability of post-surgery distant metastasis-free survival, aggressive treatment should be provided. Such aggressive treatment may include any treatment regimen beside surgery and hormone deprivation therapy (using blockers of estrogen receptor, or aromatase inhibitors). Thus, in one aspect, the present disclosure provides a method for treating breast cancer, which comprises determining the prognosis of breast cancer in a patient in the methods described above, and recommending, prescribing or administering a particular treatment regimen (e.g., a treatment regimen comprising chemotherapy) based in part on the determined prognosis.

For many breast cancer patients neoadjuvant chemotherapy is administered. In such cases, chemotherapy is given to the patient before any resection, generally in the hope that the tumor will shrink without the need for surgery. Neoadjuvant chemotherapy can cure some patients but the toxic drugs can be debilitating and expensive, making the decision whether to undertake neoadjuvant chemotherapy difficult. For patients identified according to the methods described above as having a poor prognosis (e.g., increased probability of recurrence or decreased probability of post-surgery distant metastasis-free survival), aggressive treatment comprising neoadjuvant chemotherapy may be provided. See Example 2, below. Thus, in one aspect, the present disclosure provides a method for treating breast cancer, which comprises determining the prognosis of breast cancer in a patient who has not yet had surgical resection of the tumor as described herein, and recommending, prescribing or administering a treatment regimen comprising neoadjuvant chemotherapy based at least in part on the determined prognosis. Unless stated otherwise (or unless context clearly indicates otherwise), “chemotherapy” as used herein means adjuvant and/or neoadjuvant chemotherapy.

In one embodiment, the breast cancer treatment method includes: determining in a sample from the patient the expression of a plurality of test genes comprising at least 6, 8, 10 or 15 or more cell-cycle genes and at least 6, 8, 10 or 15 or more genes selected from BCRGs, TCRGs, HLAGs, and OCPGs, determining in the same or different sample from the patient the expression of the ABCC5 gene or the PGR gene or both, and recommending, prescribing or administering a particular treatment regimen (e.g., a treatment regimen comprising chemotherapy) based in part on the determined expression of the plurality of test genes, as well as the determined ABCC5 and/or PGR expression. In some embodiments, the method further comprises administering to the patient a non-hormone-blocking therapy agent or radiotherapy. “Hormone-blocking therapy” as generally understood in the art means drugs that block the estrogen receptor, e.g., tamoxifen, or block the production of estrogen, e.g., using aromatase inhibitors such as anastrozole (Arimidex) or letrozole (Femara). Non-hormone-blocking therapy agents suitable for breast cancer adjuvant therapy are known in the art and may include, e.g., cyclophosphamide, doxorubicin (Adriamycin), taxane, methotrexate, fluorouracil, and monoclonal antibodies such as Trastuzumab.

As used herein, a patient has an “increased likelihood” of some clinical feature or outcome (e.g., response) if the probability of the patient having the feature or outcome exceeds some reference probability or value. The reference probability may be the probability of the feature or outcome across the general relevant patient population. For example, if the probability of cancer recurrence after surgery in the general breast cancer patient population (or some specific subpopulation) is X % and a particular patient has been determined by the methods of the present disclosure to have a probability of recurrence of Y %, and if Y>X, then the patient has an “increased likelihood” of response. Alternatively, as discussed above, a threshold or reference value may be determined and a particular patient's probability of response may be compared to that threshold or reference. Because predicting outcome is a prognostic endeavor, “predicting prognosis” will sometimes be used herein to refer to predicting recurrence or survival.

The results of any analyses according to the disclosure will often be communicated to physicians, genetic counselors and/or patients (or other interested parties such as researchers) in a transmittable form that can be communicated or transmitted to any of the above parties. Such a form can vary and can be tangible or intangible. The results can be embodied in descriptive statements, diagrams, photographs, charts, images or any other visual forms. For example, graphs showing expression or activity level or sequence variation information for various genes can be used in explaining the results. Diagrams showing such information for additional target gene(s) are also useful in indicating some testing results. The statements and visual forms can be recorded on a tangible medium such as papers, computer readable media such as floppy disks, compact disks, etc., or on an intangible medium, e.g., an electronic medium in the form of email or website on internet or intranet. In addition, results can also be recorded in a sound form and transmitted through any suitable medium, e.g., analog or digital cable lines, fiber optic cables, etc., via telephone, facsimile, wireless mobile phone, internet phone and the like.

Thus, the information and data on a test result can be produced anywhere in the world and transmitted to a different location. As an illustrative example, when an expression level, activity level, or sequencing (or genotyping) assay is conducted outside the United States, the information and data on a test result may be generated, cast in a transmittable form as described above, and then imported into the United States. Accordingly, the present disclosure also encompasses a method for producing a transmittable form of information on at least one of (a) expression level or (b) activity level for at least one patient sample. The method comprises the steps of (1) determining at least one of (a) or (b) above according to methods of the present disclosure; and (2) embodying the result of the determining step in a transmittable form. The transmittable form is a product of such a method.

Techniques for analyzing such expression, activity, and/or sequence data (indeed any data obtained according to the disclosure) will often be implemented using hardware, software or a combination thereof in one or more computer systems or other processing systems capable of effectuating such analysis.

Thus, the present disclosure further provides a system for determining gene expression in a sample, comprising: (1) a sample analyzer for determining the expression levels of a panel of genes in a sample (e.g., a tumor sample) including at least 2, 4, 6, 8 or 10 cell-cycle genes and at least 2, 4, 6, 8 or 10 genes selected from BCRGs, TCRGs, HLAGs, and OCPGs, wherein the sample analyzer contains the sample which is from a patient having breast cancer, or mRNA molecules from the patient sample or cDNA molecules from mRNA expressed from the panel of genes; (2) a first computer program for (a) receiving gene expression data on at least 4 test genes selected from the panel of genes, (b) weighting the determined expression of each of the test genes, and (c) combining the weighted expression to provide a test value, wherein at least 20%, 50%, at least 75% or at least 90% of the test genes are genes selected from cell-cycle genes, BCRGs, TCRGs, HLAGs, and OCPGs (or wherein the genes are weighted to contribute at least 50%, 60%, 70%, 80%, 90%, 95% or 100% of the test value), and optionally wherein the test genes include ABCC5 or PGR or both; and (3) a second computer program for comparing the test value to one or more reference values each associated with (a) a predetermined degree of risk of cancer recurrence or progression of cancer and/or (b) a predetermined degree of likelihood of response to a particular treatment regimen (e.g., treatment regimen comprising chemotherapy). In some embodiments, the system further comprises a display module displaying the comparison between the test value to the one or more reference values, or displaying a result of the comparing step.

In some embodiments, the amount of RNA transcribed from the panel of genes including test genes is measured in the sample. In addition, the amount of RNA of one or more housekeeping genes in the sample is also measured, and used to normalize or calibrate the expression of the test genes, as described above.

In some embodiments, the plurality of test genes includes at least 2, 3 or 4 cell-cycle genes and at least 2, 3, or 4 genes selected from BCRGs, TCRGs, HLAGs, and OCPGs, together which constitute at least 50%, 75% or 80% of the plurality of test genes, and preferably 100% of the plurality of test genes. In some embodiments, the plurality of test genes includes at least 5, 6 or 7, or at least 8 cell-cycle genes and at least 5, 6, or 7 or at least 8 genes selected from BCRGs, TCRGs, HLAGs, and OCPGs, together which constitute at least 20%, 25%, 30%, 40%, 50%, 60%, 70%, 75%, 80% or 90% of the plurality of test genes, and preferably 100% of the plurality of test genes.

In some other embodiments, the plurality of test genes includes at least 8, 10, 12, 15, 20, 25 or 30 genes selected from BCRGs, TCRGs, HLAGs and OCPGs, together which constitute at least 20%, 25%, 30%, 40%, 50%, 60%, 70%, 75%, 80% or 90% of the plurality of test genes, and preferably 100% of the plurality of test genes.

In some other embodiments, in addition to the BCRGs, TCRGs, HLAGs, and OCPGs, the plurality of test genes includes at least 8, 10, 12, 15, 20, 25 or 30 cell-cycle genes, together which constitute at least 20%, 25%, 30%, 40%, 50%, 60%, 70%, 75%, 80% or 90% of the plurality of test genes, and preferably 100% of the plurality of test genes.

The sample analyzer can be any instrument useful in determining gene expression, including, e.g., a sequencing machine, a real-time PCR machine, and a microarray instrument.

The computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™- or JavaScript™-enabled browsers such as HotJava™, Microsoft™ Explorer™, or Netscape™ can be used. When active content web pages are used, they may include Java™ applets or ActiveX™ controls or other active content technologies.

The analysis function can also be embodied in computer program products and used in the systems described above or other computer- or internet-based systems. Accordingly, another aspect of the present disclosure relates to a computer program product comprising a computer-usable medium having computer-readable program codes or instructions embodied thereon for enabling a processor to carry out gene status analysis. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

Thus one aspect of the present disclosure provides a system for determining whether a patient has increased likelihood of response to a particular treatment regimen. Generally speaking, the system comprises (1) computer program for receiving, storing, and/or retrieving a patient's ISG, OCPG, and/or CCG status data (e.g., expression level, activity level, variants), optionally ABCC5 status data, optionally PGR status data, and optionally clinical parameter data (e.g., age, tumor size, node status); (2) computer program for querying this patient data; (3) computer program for concluding whether there is an increased likelihood of recurrence based on this patient data; and optionally (4) computer program for outputting/displaying this conclusion. In some embodiments this means for outputting the conclusion may comprise a computer program for informing a health care professional of the conclusion.

Thus in some embodiments the disclosure provides a method comprising: accessing information on a patient's ISG status, OCPGs status, optionally CCP status, optionally ABCC5 status, optionally PGR status, optionally clinical variable or score status is stored in a computer-readable medium; querying this information to determine whether a sample obtained from the patient shows increased expression of a plurality of test genes comprising at least 2 ISGs or OCPGs (e.g., a test value representing the expression of this plurality of test genes that is weighted such that ISGs and or OCPGs contribute at least 50% to the test value, such test value being higher than some reference value); outputting [or displaying] the quantitative or qualitative (e.g., “increased”) likelihood that the patient will respond to a particular treatment regimen. As used herein in the context of computer-implemented embodiments of the disclosure, “displaying” means communicating any information by any sensory means. Examples include, but are not limited to, visual displays, e.g., on a computer screen or on a sheet of paper printed at the command of the computer, and auditory displays, e.g., computer generated or recorded auditory expression of a patient's genotype.

The practice of the present disclosure may also employ conventional biology methods, software and systems. Computer software products of the disclosure typically include computer readable media having computer-executable instructions for performing the logic steps of the method of the disclosure. Suitable computer readable medium include floppy disk, CD-ROM/DVD/DVD-ROM, hard-disk drive, flash memory, ROM/RAM, magnetic tapes and etc. Basic computational biology methods are described in, for example, Setubal et al., INTRODUCTION TO COMPUTATIONAL BIOLOGY METHODS (PWS Publishing Company, Boston, 1997); Salzberg et al. (Ed.), COMPUTATIONAL METHODS IN MOLECULAR BIOLOGY, (Elsevier, Amsterdam, 1998); Rashidi & Buehler, BIOINFORMATICS BASICS: APPLICATION IN BIOLOGICAL SCIENCE AND MEDICINE (CRC Press, London, 2000); and Ouelette & Bzevanis, BIOINFORMATICS: A PRACTICAL GUIDE FOR ANALYSIS OF GENE AND PROTEINS (Wiley & Sons, Inc., 2^{nd} ed., 2001); see also, U.S. Pat. No. 6,420,108.

The present disclosure may also make use of various computer program products and software for a variety of purposes, such as probe design, management of data, analysis, and instrument operation. See U.S. Pat. Nos. 5,593,839; 5,795,716; 5,733,729; 5,974,164; 6,066,454; 6,090,555; 6,185,561; 6,188,783; 6,223,127; 6,229,911 and 6,308,170. Additionally, the present disclosure may have embodiments that include methods for providing genetic information over networks such as the Internet as shown in U.S. Ser. No. 10/197,621 (U.S. Pub. No. 20030097222); Ser. No. 10/063,559 (U.S. Pub. No. 20020183936), Ser. No. 10/065,856 (U.S. Pub. No. 20030100995); Ser. No. 10/065,868 (U.S. Pub. No. 20030120432); Ser. No. 10/423,403 (U.S. Pub. No. 20040049354).

Techniques for analyzing such expression, activity, and/or sequence data (indeed any data obtained according to the disclosure) will often be implemented using hardware, software or a combination thereof in one or more computer systems or other processing systems capable of effectuating such analysis.

Thus one aspect of the present disclosure provides systems related to the above methods of the disclosure. In one embodiment the disclosure provides a system for determining a patient's prognosis and/or whether a patient will respond to a particular treatment regimen, comprising:

(1) a sample analyzer for determining the expression levels in a sample of a plurality of test genes including at least 4 genes selected from BCRGs, TCRGs, HLAGs, and OCPGs, and in addition optionally including CCGS and/or ABCC5 or PGR or both, wherein the sample analyzer contains the sample, RNA from the sample and expressed from the panel of genes, or DNA synthesized from said RNA;

(2) a first computer program for

(a) receiving gene expression data on said plurality of test genes,

(b) weighting the determined expression of each of the test genes with a predefined coefficient, and

(c) combining the weighted expression to provide a test value, wherein the combined weight given to said at least 4 genes selected from BCRGs, TCRGs, HLAGs, and OCPGs and in addition optionally including the CCGs and/or ABCC5 or PGR or both, is at least 10% (or 20%, 30%, 40%, 50%, 60%, 70%, 80%, 90%) of the total weight given to the expression of all of said plurality of test genes; and

(3) a second computer program for comparing the test value to one or more reference values each associated with a predetermined likelihood of recurrence or progression or a predetermined likelihood of response to a particular treatment regimen.

In some embodiments at least 5%, 10%, 20%, 50%, 75%, or 90% of said plurality of test genes are selected from BCRGs, TCRGs, HLAGs, and OCPGs. In some embodiments the sample analyzer contains reagents for determining the expression levels in the sample of said panel of genes including at least 4 genes chosen from BCRGs, TCRGs, HLAGs and OCPGs and in addition optionally including the CCGs, and/or ABCC5 or PGR or both.

In another embodiment the disclosure provides a system for determining gene expression in a sample (e.g., tumor sample), comprising: (1) a sample analyzer for determining the expression levels of a panel of genes in a sample including at least genes selected from BCRGs, TCRGs, HLAGs, and OCPGs, and in addition optionally including the CCGs, and/or ABCC5 or PGR or both, wherein the sample analyzer contains the sample which is from a patient having breast cancer, RNA from the sample and expressed from the panel of genes, or DNA synthesized from said RNA; (2) a first computer program for (a) receiving gene expression data on at least 4 test genes selected from the panel of genes, (b) weighting the determined expression of each of the test genes with a predefined coefficient, and (c) combining the weighted expression to provide a test value, wherein the combined weight given to said at least 4 ISGs and OCPGs is at least 10% (or 20%, 30%, 40% 50%, 60%, 70%, 80%, 90%) of the total weight given to the expression of all of said plurality of test genes; and (3) a second computer program for comparing the test value to one or more reference values each associated with a predetermined degree of risk of cancer recurrence or progression of breast cancer. In some embodiments at least 20%, 50%, 75%, or 90% of said plurality of test genes are ISGs and/or OCPGs. In some embodiments the system comprises a computer program for determining the patient's prognosis and/or determining (including quantifying) the patient's degree of risk of cancer recurrence or progression based at least in part on the comparison of the test value with said one or more reference values.

In some embodiments, the system further comprises a display module displaying the comparison between the test value and the one or more reference values, or displaying a result of the comparing step, or displaying the patient's prognosis and/or degree of risk of cancer recurrence or progression.

In a preferred embodiment, the amount of RNA transcribed from the panel of genes including test genes (and/or DNA reverse transcribed therefrom) is measured in the sample. In addition, the amount of RNA of one or more housekeeping genes in the sample (and/or DNA reverse transcribed therefrom) is also measured, and used to normalize or calibrate the expression of the test genes, as described above.

In some embodiments, the plurality of test genes includes at least 2, 3 or 4 ISGs or OCPGs, which constitute at least 50%, 75%, 80%, 90% or 95% of the plurality of test genes of the plurality of test genes. In some embodiments, the plurality of test genes includes at least 5, 6 or 7, or at least 8 ISGs, OCPGs, which constitute at least 20%, 25%, 30%, 40%, 50%, 60%, 70%, 75%, 80% or 90% of the plurality of test genes. Thus in some embodiments the plurality of test genes comprises at least some number of ISGs and OCPGs (e.g., at least 3, 4, 5, 6, 7, 8, 9, 10, 15, 20, 25, 30, 35, 40, 45, 50 or more ISGs and OCPGs) and this plurality of ISGs and OCPGs comprises the top 2, 3, 4, 5, 6, 7, 8, 9, 10, 11, 12, 13, 14, 15, 20, 25, 30, 35, 40 or more ISGs or OCPGs listed in any of Tables 1, 6A, 6B, 8, 9, 30, 31, 32, or 33. In some embodiments the plurality of test genes comprises at least some number of ISGs and OCPGs (e.g., at least 3, 4, 5, 6, 7, 8, 9, 10, 15, 20, 25, 30, 35, 40, 45, 50 or more ISGs and OCPGs) and this plurality of ISGs and OCPGs comprises at least 1, 2, 3, 4, 5, 6, 7, 8, 9, 10, 15, or 20 of the following genes: CD38, IRF4, CKAP2, POLR2H, NHLH2, RPL5, PECAM1, CNOT2, SELL, CACNB3, ITGB2, HSD11B1. CCL19, IGVH, SIX1, CCL5, DLAT, EVI2B, STAT5A, CD247. In some embodiments the plurality of test genes comprises beside at least some number of ISG and OCPGs (e.g., at least 3, 4, 5, 6, 7, 8, 9, 10, 15, 20, 25, 30, 35, 40, 45, 50 or more ISGs and OCPGs) and this plurality of ISGs and OCPGs comprises any one, two, three, four, five, six, seven, eight, nine, or ten or all of gene numbers 1 & 2, 1 to 3, 1 to 4, 1 to 5, 1 to 6, 1 to 7, 1 to 8, 1 to 9, or 1 to 10 of any of Tables 1, 6A, 6B, 8, 9, 30, 31, 32, or 33. In some embodiments the plurality of test genes comprises at least some number of ISGs and OCPGss (e.g., at least 3, 4, 5, 6, 7, 8, 9, 10, 15, 20, 25, 30, 35, 40, 45, 50 or more ISGs and OCPGs) and this plurality of ISGs and OCPGs comprises any one, two, three, four, five, six, seven, eight, or nine or all of gene numbers 2 & 3, 2 to 4, 2 to 5, 2 to 6, 2 to 7, 2 to 8, 2 to 9, or 2 to 10 of any of Tables 1, 6A, 6B, 8, 9, 30, 31, 32, or 33. In some embodiments the plurality of test genes comprises at least some number of ISGs and OCPGs (e.g., at least 3, 4, 5, 6, 7, 8, 9, 10, 15, 20, 25, 30, 35, 40, 45, 50 or more ISGs and OCPGs) and this plurality of ISGs and OCPGs comprises any one, two, three, four, five, six, seven, or eight or all of gene numbers 3 & 4, 3 to 5, 3 to 6, 3 to 7, 3 to 8, 3 to 9, or 3 to 10 of any of Tables 1, 6A, 6B, 8, 9, 30, 31, 32, or 33. In some embodiments the plurality of test genes comprises at least some number of ISGs and OCPGs (e.g., at least 3, 4, 5, 6, 7, 8, 9, 10, 15, 20, 25, 30, 35, 40, 45, 50 or more ISGs and OCPGsGs) and this plurality of CCGs comprises any one, two, three, four, five, six, or seven or all of gene numbers 4 & 5, 4 to 6, 4 to 7, 4 to 8, 4 to 9, or 4 to 10 of any of Tables 1, 6A, 6B, 8, 9, 30, 31, 32, or 33. In some embodiments the plurality of test genes comprises at least some number of ISGs and OCPGs (e.g., at least 3, 4, 5, 6, 7, 8, 9, 10, 15, 20, 25, 30, 35, 40, 45, 50 or more ISGs and OCPsGs) and this plurality of CCGs comprises any one, two, three, four, five, six, seven, eight, nine, 10, 11, 12, 13, 14, or 15 or all of gene numbers 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Tables 1, 6A, 6B, 8, 9, 30, 31, 32, or 33.

In some embodiments, the plurality of test genes includes at least 2, 3 or 4 CCGs in addition to ISGs or OCPGs, which constitute at least 50%, 75%, 80%, 90% or 95% of the plurality of test genes of the plurality of test genes. In some embodiments, the plurality of test genes includes at least 5, 6 or 7, or at least 8 CCGs in addition to ISGs, OCPGs, which constitute at least 20%, 25%, 30%, 40%, 50%, 60%, 70%, 75%, 80% or 90% of the plurality of test genes. Thus in some embodiments the plurality of test genes comprises at least some number of CCGs, ISGs and OCPGs (e.g., at least 3, 4, 5, 6, 7, 8, 9, 10, 15, 20, 25, 30, 35, 40, 45, 50 or more CCGs, ISGs and OCPGs) and this plurality of CCGs, ISGs and OCPGs comprises the top 2, 3, 4, 5, 6, 7, 8, 9, 10, 11, 12, 13, 14, 15, 20, 25, 30, 35, 40 or more genes listed in any of Tables 1, 6A, 6B, 8, 9, 30, 31, 32, or 33, and/or any of Tables 10, 11, 12, 13, 14, 15, 19, 20, 21, 22, 23, 24, or 25. In some embodiments the plurality of test genes comprises at least some number of CCGs (e.g., at least 3, 4, 5, 6, 7, 8, 9, 10, 15, 20, 25, 30, 35, 40, 45, 50 or more CCGs) in addition to at least some number of ISGs and OCPGs (e.g., at least 3, 4, 5, 6, 7, 8, 9, 10, 15, 20, 25, 30, 35, 40, 45, 50 or more ISGs and OCPGs) and this plurality of CCGs, ISGs and OCPGs comprises at least 1, 2, 3, 4, 5, 6, 7, 8, 9, 10, 15, or 20 of the following genes: ASPM, BIRC5, BUB1B, CCNB2, CDC2, CDC20, CDCA8, CDKN3, CENPF, DLAGP5, FOXM1, KIAA010, KIF11, KIF2C, KIF4A, MCM10, NUSAP1, PRC1, RACGAP1, and TPX2, and at least 1, 2, 3, 4, 5, 6, 7, 8, 9, 10, 15, or 20 of the following genes: CD38, IRF4, CKAP2, POLR2H, NHLH2, RPL5, PECAM1, CNOT2, SELL, CACNB3, ITGB2, HSD11B1. CCL19, IGVH, SIX1, CCL5, DLAT, EVI2B, STAT5A, CD247. In some embodiments the plurality of test genes comprises at least some number of CCGs (e.g., at least 3, 4, 5, 6, 7, 8, 9, 10, 15, 20, 25, 30, 35, 40, 45, 50 or more CCGs) in addition to at least some number of ISG and OCPGs (e.g., at least 3, 4, 5, 6, 7, 8, 9, 10, 15, 20, 25, 30, 35, 40, 45, 50 or more ISGs and OCPGs) and this plurality of ISGs and OCPG, and CCGs comprises any one, two, three, four, five, six, seven, eight, nine, or ten or all of gene numbers 1 & 2, 1 to 3, 1 to 4, 1 to 5, 1 to 6, 1 to 7, 1 to 8, 1 to 9, or 1 to 10 of any of Tables 1, 6A, 6B, 8, 9, 30, 31, 32, or 33, and/or any of Tables 10, 11, 12, 13, 14, 15, 19, 20, 21, 22, 23, 24, or 25. In some embodiments the plurality of test genes comprises at least some number of CCGs (e.g., at least 3, 4, 5, 6, 7, 8, 9, 10, 15, 20, 25, 30, 35, 40, 45, 50 CCGs) in addition to the at least some number of ISGs and OCPGs (e.g., at least 3, 4, 5, 6, 7, 8, 9, 10, 15, 20, 25, 30, 35, 40, 45, 50 or more ISGs and OCPGs) and this plurality of ISGs and OCPGs, and CCGs comprises any one, two, three, four, five, six, seven, eight, or nine or all of gene numbers 2 & 3, 2 to 4, 2 to 5, 2 to 6, 2 to 7, 2 to 8, 2 to 9, or 2 to 10 of any of Tables 1, 6A, 6B, 8, 9, 30, 31, 32, or 33, and/or any of Tables 10, 11, 12, 13, 14, 15, 19, 20, 21, 22, 23, 24, or 25. In some embodiments the plurality of test genes comprises at least some number of CCGs (e.g., at least 3, 4, 5, 6, 7, 8, 9, 10, 15, 20, 25, 30, 35, 40, 45, 50 or more CCGs) in addition to at least some number of ISGs and OCPGs (e.g., at least 3, 4, 5, 6, 7, 8, 9, 10, 15, 20, 25, 30, 35, 40, 45, 50 or more ISGs and OCPGs) and this plurality of CCGs, ISGs and OCPGs comprises any one, two, three, four, five, six, seven, or eight or all of gene numbers 3 & 4, 3 to 5, 3 to 6, 3 to 7, 3 to 8, 3 to 9, or 3 to 10 of any of Tables 1, 6A, 6B, 8, 9, 30, 31, 32, or 33, and/or any of Tables 10, 11, 12, 13, 14, 15, 19, 20, 21, 22, 23, 24, or 25. In some embodiments the plurality of test genes comprises at least some number of CCGs (e.g., at least 3, 4, 5, 6, 7, 8, 9, 10, 15, 20, 25, 30, 35, 40, 45, 50 or more CCGs) in addition to at least some number of ISGs and OCPGs (e.g., at least 3, 4, 5, 6, 7, 8, 9, 10, 15, 20, 25, 30, 35, 40, 45, 50 or more ISGs and OCPGs) and this plurality of ISGs, OCPGs and CCGs comprises any one, two, three, four, five, six, or seven or all of gene numbers 4 & 5, 4 to 6, 4 to 7, 4 to 8, 4 to 9, or 4 to 10 of any of Tables 1, 6A, 6B, 8, 9, 30, 31, 32, or 33, and/or any of Tables 10, 11, 12, 13, 14, 15, 19, 20, 21, 22, 23, 24, or 25. In some embodiments the plurality of test genes comprises at least some number of CCGs (e.g., at least 3, 4, 5, 6, 7, 8, 9, 10, 15, 20, 25, 30, 35, 40, 45, 50 or more CCGs) in addition to at least some number of ISGs and OCPGs (e.g., at least 3, 4, 5, 6, 7, 8, 9, 10, 15, 20, 25, 30, 35, 40, 45, 50 or more ISGs and OCPsGs) and this plurality of ISGs, OCPs and CCGs comprises any one, two, three, four, five, six, seven, eight, nine, 10, 11, 12, 13, 14, or 15 or all of gene numbers 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Tables 1, 6A, 6B, 8, 9, 30, 31, 32, or 33, and/or any of Tables 10, 11, 12, 13, 14, 15, 19, 20, 21, 22, 23, 24, or 25.

In some embodiments, the plurality of test genes includes at least 2, 3 or 4 ISGs and OCPGs and ABCC5 or PGR or both, which constitute at least 50%, 75%, 80%, 90% or 95% of the plurality of test genes of the plurality of test genes. In some embodiments, the plurality of test genes includes at least 5, 6 or 7, or at least 8 ISGs and OCGPs, and ABCC5 or PGR or both, which constitute at least 20%, 25%, 30%, 40%, 50%, 60%, 70%, 75%, 80% or 90% of the plurality of test genes. Thus in some embodiments the plurality of test genes comprises in addition to ABCC5 or PGR or both, at least some number of ISGs and OCPGs (e.g., at least 3, 4, 5, 6, 7, 8, 9, 10, 15, 20, 25, 30, 35, 40, 45, 50 or more ISGs and OCPGs) and this plurality of ISGs and OCPGs comprises the top 2, 3, 4, 5, 6, 7, 8, 9, 10, 11, 12, 13, 14, 15, 20, 25, 30, 35, 40 or more ISGs and OCPGs listed in any of Tables 1, 6A, 6B, 8, 9, 30, 31, 32, or 33. In some embodiments the plurality of test genes comprises in addition to ABCC5 or PGR or both, at least some number of ISGs and OCPGs (e.g., at least 3, 4, 5, 6, 7, 8, 9, 10, 15, 20, 25, 30, 35, 40, 45, 50 or more ISGs and OCPGs) and this plurality of ISG and OCPGs comprises at least 1, 2, 3, 4, 5, 6, 7, 8, 9, 10, 15, or 20 of the following genes CD38, IRF4, CKAP2, POLR2H, NHLH2, RPL5, PECAM1, CNOT2, SELL, CACNB3, ITGB2, HSD11B1. CCL19, IGVH, SIX1, CCL5, DLAT, EVI2B, STAT5A, CD247. In some embodiments the plurality of test genes comprises beside ABCC5 or PGR or both, at least some number of ISGs and OCPGs (e.g., at least 3, 4, 5, 6, 7, 8, 9, 10, 15, 20, 25, 30, 35, 40, 45, 50 or more ISGs and OCPSGs) and this plurality of ISGs and OCPGs comprises any one, two, three, four, five, six, seven, eight, nine, or ten or all of gene numbers 1 & 2, 1 to 3, 1 to 4, 1 to 5, 1 to 6, 1 to 7, 1 to 8, 1 to 9, or 1 to 10 of any of Tables 1, 6A, 6B, 8, 9, 30, 31, 32, or 33, and/or any of Tables 10, 11, 12, 13, 14, 15, 19, 20, 21, 22, 23, 24, or 25. In some embodiments the plurality of test genes comprises in addition to ABCC5 or PGR or both, at least some number of ISGs and OCPGs (e.g., at least 3, 4, 5, 6, 7, 8, 9, 10, 15, 20, 25, 30, 35, 40, 45, 50 or more ISGs and OCPGs) and this plurality of ISGs and OCPGs comprises any one, two, three, four, five, six, seven, eight, or nine or all of gene numbers 2 & 3, 2 to 4, 2 to 5, 2 to 6, 2 to 7, 2 to 8, 2 to 9, or 2 to 10 of any of Tables 1, 6A, 6B, 8, 9, 30, 31, 32, or 33. In some embodiments the plurality of test genes comprises in addition to ABCC5 or PGR or both, at least some number of ISGs and OCPGs (e.g., at least 3, 4, 5, 6, 7, 8, 9, 10, 15, 20, 25, 30, 35, 40, 45, 50 or more ISGs and OCPGs) and this plurality of ISGs and OCPGs comprises any one, two, three, four, five, six, seven, or eight or all of gene numbers 3 & 4, 3 to 5, 3 to 6, 3 to 7, 3 to 8, 3 to 9, or 3 to 10 of any of Tables 1, 6A, 6B, 8, 9, 30, 31, 32, or 33. In some embodiments the plurality of test genes comprises in addition to ABCC5 or PGR or both, at least some number of ISGs and OCPGs (e.g., at least 3, 4, 5, 6, 7, 8, 9, 10, 15, 20, 25, 30, 35, 40, 45, 50 or more ISGs and OCPGs) and this plurality of ISGs and OCPGs comprises any one, two, three, four, five, six, or seven or all of gene numbers 4 & 5, 4 to 6, 4 to 7, 4 to 8, 4 to 9, or 4 to 10 of any of Tables 1, 6A, 6B, 8, 9, 30, 31, 32, or 33. In some embodiments the plurality of test genes comprises in addition to ABCC5 or PGR or both, at least some number of ISGs and OCPGs (e.g., at least 3, 4, 5, 6, 7, 8, 9, 10, 15, 20, 25, 30, 35, 40, 45, 50 or more ISGs and OCPGs) and this plurality of ISGs and OCPGs comprises any one, two, three, four, five, six, seven, eight, nine, 10, 11, 12, 13, 14, or 15 or all of gene numbers 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Tables 1, 6A, 6B, 8, 9, 30, 31, 32, or 33.

In some other embodiments, the plurality of test genes includes at least 8, 10, 12, 15, 20, 25 or 30 ISGs and OCPGs, which constitute at least 20%, 25%, 30%, 40%, 50%, 60%, 70%, 75%, 80% or 90% of the plurality of test genes, and preferably 100% of the plurality of test genes. In some other embodiments, the plurality of test genes in addition to some number of ISGs and OCPs includes in at least 8, 10, 12, 15, 20, 25 or 30 CCGs, which constitute at least 20%, 25%, 30%, 40%, 50%, 60%, 70%, 75%, 80% or 90% of the plurality of test genes. In some other embodiments, the plurality of test genes in addition to some number of ISGs and OCPs includes ABCC5 or PGR or both.

The sample analyzer can be any instrument useful in determining gene expression, including, e.g., a sequencing machine (e.g., Illumina HiSeq™, Ion Torrent PGM, ABI SOLiD™ sequencer, PacBio RS, Helicos Heliscope™, etc.), a real-time PCR machine (e.g., ABI 7900, Fluidigm BioMark™, etc.), a microarray instrument, etc.

In one aspect, the present disclosure provides methods of treating a cancer patient comprising obtaining status information (e.g., expression) for a plurality of test genes (e.g., the ISGs, and OCPGs in Table 1, 2, 3, 5, 6a, or 6b,), and recommending, prescribing or administering a treatment for the cancer patient based on the test gene status. For example, the disclosure provides a method of treating a cancer patient comprising:

(1) determining the expression of a plurality of test genes, wherein said plurality of test genes comprises at least 4 (or 5, 6, 7, 8, 9, 10, 15, 20, 30 or more) ISGs and OCPGs;

(2) based at least in part on the determination in step (1), recommending, prescribing or administering either

(a) a treatment regimen comprising chemotherapy (e.g., adjuvant chemotherapy) if the patient has increased expression of wpOCGs (e.g., and ISGs and OCPGs are weighted to contribute at least 50% to the determination of increased expression of the plurality of test genes), or

(b) a treatment regimen not comprising chemotherapy if the patient does not have increased expression of wpOCGs (e.g., and ISGs and OCPGs are weighted to contribute at least 50% to the determination of increased expression of the plurality of test genes), or

(c) a treatment regimen comprising chemotherapy (e.g., adjuvant chemotherapy) if the patient has a decreased expression of ISGs (e.g., and ISGs and OCPGs are weighted to contribute at least 50% to the determination of increased expression of the plurality of test genes), or

(d) a treatment regimen not comprising chemotherapy if the patient has an increased expression of ISGs (e.g., and ISGs and OCPGs are weighted to contribute at least 50% to the determination of increased expression of the plurality of test genes).

In one aspect, the present disclosure provides methods of treating a cancer patient comprising obtaining the information status of a plurality of test genes (e.g., the ISGs, OCPGs and CCGs in Table 1, 2, 3, 5, 6, or 7), and recommending, prescribing or administering a treatment for the cancer patient based on the test gene status. For example, the disclosure provides a method of treating a cancer patient comprising:

(1) determining the expression of a plurality of test genes, wherein said plurality of test genes comprises at least 4 (or 5, 6, 7, 8, 9, 10, 15, 20, 30 or more) ISGs and OCPGs and at least 4 (or 5, 6, 7, 8, 9, 10, 15, 20, 30 or more) CCGs;

(2) based at least in part on the determination in step (1), recommending, prescribing or administering either

(a) a treatment regimen comprising chemotherapy (e.g., adjuvant chemotherapy) if the patient has increased expression of CCGs and wpOCGs (e.g., and ISGs and OCPGs are weighted to contribute at least 50% to the determination of increased expression of the plurality of test genes), or

(b) a treatment regimen not comprising chemotherapy if the patient does not have increased expression of the CCGs and wpOCGs (e.g., and ISGs and OCPGs are weighted to contribute at least 50% to the determination of increased expression of the plurality of test genes).

(c) a treatment regimen comprising chemotherapy (e.g., adjuvant chemotherapy) if the patient has a decreased expression of ISGs (e.g., and ISGs and OCPGs are weighted to contribute at least 50% to the determination of increased expression of the plurality of test genes), or

(d) a treatment regimen not comprising chemotherapy if the patient has an increased expression of ISGs (e.g., and ISGs and OCPGs are weighted to contribute at least 50% to the determination of increased expression of the plurality of test genes).

In one aspect, the present disclosure provides methods of treating a cancer patient comprising obtaining ISG and OCPG status information (e.g., the ISGs and OCPGs in Table 1, 2, 3, 5, 6a or 6b), and recommending, prescribing or administering a treatment for the cancer patient based on the ISG and OCPGs status. For example, the disclosure provides a method of treating a cancer patient comprising:

(1) determining the expression of ABCC5 or PGR or both in addition to a plurality of test genes, wherein said plurality of test genes comprises at least 4 (or 5, 6, 7, 8, 9, 10, 15, 20, 30 or more) ISGs and OCPGs;

(2) based at least in part on the determination in step (1), recommending, prescribing or administering either

(a) a treatment regimen comprising chemotherapy (e.g., adjuvant chemotherapy) if the patient has increased expression of wpOCGs (e.g., and ISGs and OCPGs are weighted to contribute at least 50% to the determination of increased expression of the plurality of test genes), or

(b) a treatment regimen not comprising chemotherapy if the patient does not have increased expression of wpOCGs (e.g., and ISGs and OCPGs are weighted to contribute at least 50% to the determination of increased expression of the plurality of test genes), or

(c) a treatment regimen comprising chemotherapy (e.g., adjuvant chemotherapy) if the patient has a decreased expression of ISGs (e.g., and ISGs and OCPGs are weighted to contribute at least 50% to the determination of increased expression of the plurality of test genes), or

(d) a treatment regimen not comprising chemotherapy if the patient has an increased expression of ISGs (e.g., and ISGs and OCPGs are weighted to contribute at least 50% to the determination of increased expression of the plurality of test genes).

In one aspect, the disclosure provides compositions for use in the above methods. Such compositions include, but are not limited to, nucleic acid probes hybridizing to, an ISG or an OCPG including but not limited to an ISG or OCPGsCCG listed in any of Tables 1, 2, 3, 5, 6a, or 6b (or to any nucleic acids encoded thereby or complementary thereto); nucleic acid primers and primer pairs suitable for selectively amplifying all or a portion of the ISG or OCPG or any nucleic acids encoded thereby; antibodies binding immunologically to a polypeptide encoded by the ISG or OCPG; probe sets comprising a plurality of said nucleic acid probes, nucleic acid primers, antibodies, and/or polypeptides; microarrays comprising any of these; kits comprising any of these; etc. In some aspects, the disclosure provides computer methods, systems, software and/or modules for use in the above methods. In some embodiments, such compositions include nucleic acid probes hybridizing to, ABCC5 or PGR or both, nucleic acid primers and primer pairs suitable for selectively amplifying all or a portion of ABCC5 or PGR or both, or antibodies binding immunologically to a polypeptide encoded by ABCC5 or PGR or both.

In one aspect, the disclosure provides compositions for use in the above methods. Such compositions include, but are not limited to, nucleic acid probes hybridizing to, a CCG, an ISG and OCPG including but not limited to an ISG, OCPGS, or CCG listed in any of Tables 1, 2, 3, 5, 6, or 7 (or to any nucleic acids encoded thereby or complementary thereto); nucleic acid primers and primer pairs suitable for selectively amplifying all or a portion of an ISG, OCPGs or CCG or any nucleic acids encoded thereby; antibodies binding immunologically to a polypeptide encoded by and ISG, OCPG or CCG; probe sets comprising a plurality of said nucleic acid probes, nucleic acid primers, antibodies, and/or polypeptides; microarrays comprising any of these; kits comprising any of these; etc. In some aspects, the disclosure provides computer methods, systems, software and/or modules for use in the above methods. In some embodiments, such compositions include nucleic acid probes hybridizing to, ABCC5 or PGR or both, nucleic acid primers and primer pairs suitable for selectively amplifying all or a portion of ABCC5 or PGR or both, or antibodies binding immunologically to a polypeptide encoded by ABCC5 or PGR or both.

In some embodiments the disclosure provides a probe comprising an isolated oligonucleotide capable of selectively hybridizing to at least one of the genes in Table 1, 2, 3, 5, 6a, 6b or 7. The terms “probe” and “oligonucleotide” (also “oligo”), when used in the context of nucleic acids, interchangeably refer to a relatively short nucleic acid fragment or sequence. The disclosure also provides primers useful in the methods of the disclosure. “Primers” are probes capable, under the right conditions and with the right companion reagents, of selectively amplifying a target nucleic acid (e.g., a target gene). In the context of nucleic acids, “probe” is used herein to encompass “primer” since primers can generally also serve as probes.

In some embodiments the disclosure provides a probe comprising an isolated oligonucleotide capable of selectively hybridizing to ABCC5 or PGR or both, and at least one of the genes in Table 1, 2, 3, 5, 6a, 6b or 7. The terms “probe” and “oligonucleotide” (also “oligo”), when used in the context of nucleic acids, interchangeably refer to a relatively short nucleic acid fragment or sequence. The disclosure also provides primers useful in the methods of the disclosure. “Primers” are probes capable, under the right conditions and with the right companion reagents, of selectively amplifying a target nucleic acid (e.g., a target gene). In the context of nucleic acids, “probe” is used herein to encompass “primer” since primers can generally also serve as probes.

The probe can generally be of any suitable size/length. In some embodiments the probe has a length from about 8 to 200, 15 to 150, 15 to 100, 15 to 75, 15 to 60, or 20 to 55 bases in length. They can be labeled with detectable markers with any suitable detection marker including but not limited to, radioactive isotopes, fluorophores, biotin, enzymes (e.g., alkaline phosphatase), enzyme substrates, ligands and antibodies, etc. See Jablonski et al., NUCLEIC ACIDS RES. (1986) 14:6115-6128; Nguyen et al., BIOTECHNIQUES (1992) 13:116-123; Rigby et al., J. MOL. BIOL. (1977) 113:237-251. Indeed, probes may be modified in any conventional manner for various molecular biological applications. Techniques for producing and using such oligonucleotide probes are conventional in the art.

Probes according to the disclosure can be used in the hybridization/amplification/detection techniques discussed above. Thus, some embodiments of the disclosure comprise probe sets suitable for use in a microarray in detecting, amplifying and/or quantitating a plurality of ISGs and OCPGs. In some embodiments the probe sets have a certain proportion of their probes directed to ISGs and OCPGs—e.g., a probe set consisting of 10%, 20%, 30%, 40%, 50%, 55%, 60%, 65%, 70%, 75%, 80%, 85%, 90%, 95%, 96%, 97%, 98%, or 99% probes specific for ISGs and OCPGsGs. In some embodiments the probe set comprises probes directed to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 60, 70, 80, 90, 100, 125, 150, 175, 200, 250, 300, 350, 400, 450, 500, 600, 700, or 800 or more, or all, of the genes in Table 1, 2, 3, 5, 6a or 6b. Such probe sets can be incorporated into high-density arrays comprising 5,000, 10,000, 20,000, 50,000, 100,000, 200,000, 300,000, 400,000, 500,000, 600,000, 700,000, 800,000, 900,000, or 1,000,000 or more different probes. In other embodiments the probe sets comprise primers (e.g., primer pairs) for amplifying nucleic acids comprising at least a portion of one or more of the ISGs and OCPGs in Table 1, 2, 3, 5, 6a or 6b.

Some embodiments of the disclosure comprise probe sets suitable for use in a microarray in detecting, amplifying and/or quantitating a plurality of CCGs in addition to ISGs and OCPGs. In some embodiments the probe sets have a certain proportion of their probes directed to CCGs in addition to ISGs and OCPGs—e.g., a probe set consisting of 10%, 20%, 30%, 40%, 50%, 55%, 60%, 65%, 70%, 75%, 80%, 85%, 90%, 95%, 96%, 97%, 98%, or 99% probes specific for ISGs, OCPGs and CCGs. In some embodiments the probe set comprises probes directed to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 60, 70, 80, 90, 100, 125, 150, 175, 200, 250, 300, 350, 400, 450, 500, 600, 700, or 800 or more, or all, of the genes in Table 1, 2, 3, 5, 6a, 6b or 7. Such probe sets can be incorporated into high-density arrays comprising 5,000, 10,000, 20,000, 50,000, 100,000, 200,000, 300,000, 400,000, 500,000, 600,000, 700,000, 800,000, 900,000, or 1,000,000 or more different probes. In other embodiments the probe sets comprise primers (e.g., primer pairs) for amplifying nucleic acids comprising at least a portion of one or more of the ISGs, OCPGs and CCGs in Table 1, 2, 3, 5, 6a, 6b or 7.

In another aspect of the present disclosure, a kit is provided for practicing the prognosis of the present disclosure. The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit includes various components useful in determining the status of one or more ISGs, OCPGS and one or more housekeeping gene markers, using the above-discussed detection techniques. For example, the kit many include oligonucleotides specifically hybridizing under high stringency to mRNA or cDNA of the genes in Table 1, 2, 3, 5, 6a or 6b. Such oligonucleotides can be used as PCR primers in RT-PCR reactions, or hybridization probes. In some embodiments the kit comprises reagents (e.g., probes, primers, and or antibodies) for determining the expression level of a panel of genes, where said panel comprises at least 25%, 30%, 40%, 50%, 60%, 75%, 80%, 90%, 95%, 99%, or 100% ISGs and OCPGs (e.g., ISGs and OCPGs in Table 1, 2, 3, 5, 6, 7, 8, or 9 or Panel A, B, C, D, E, F, or G). In some embodiments the kit consists of reagents (e.g., probes, primers, and or antibodies) for determining the expression level of no more than 2500 genes, wherein at least 5, 10, 15, 20, 30, 40, 50, 60, 70, 80, 90, 100, 120, 150, 200, 250, or more of these genes are ISGs and OCPGs (e.g., ISGs and OCPGs in Table 1, 2, 3, 5, 6a or 6b). In some embodiments the kit includes various components useful in determining the status of one or more CCGs, PGR, and or ABCC5 in addition to components useful in determining the status of one or more ISGs, OCPGS and one or more housekeeping gene markers, using the above-discussed detection techniques.

The oligonucleotides in the detection kit can be labeled with any suitable detection marker including but not limited to, radioactive isotopes, fluorophores, biotin, enzymes (e.g., alkaline phosphatase), enzyme substrates, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977). Alternatively, the oligonucleotides included in the kit are not labeled, and instead, one or more markers are provided in the kit so that users may label the oligonucleotides at the time of use.

In another embodiment of the disclosure, the detection kit contains one or more antibodies selectively immunoreactive with one or more proteins encoded by one or more ISG or OCPG or optionally any additional markers including ABCC5 or PGR or one or more CCG. Examples include antibodies that bind immunologically to a protein encoded by a gene in Table 1, 2, 3, 5, 6a or 6b. Methods for producing and using such antibodies are well-known in the art.

Various other components useful in the detection techniques may also be included in the detection kit of this disclosure. Examples of such components include, but are not limited to, Taq polymerase, deoxyribonucleotides, dideoxyribonucleotides, other primers suitable for the amplification of a target DNA sequence, RNase A, and the like. In addition, the detection kit preferably includes instructions on using the kit for practice the prognosis method of the present disclosure using human samples.

### SPECIFIC EMBODIMENTS

The following paragraphs describe numerous specific embodiments of the present disclosure.

### Embodiment 1

A method for determining likelihood of breast cancer recurrence, comprising:
(1) measuring, in a patient sample, the expression levels of a panel of genes comprising at least 3 test genes, wherein at least two of said test genes are selected from gene numbers 1 to 23 in Table 40 and at least one of said test genes is selected from gene numbers 24 to 30 in Table 40;
(2) providing a test expression score by (1) weighting the determined expression of each gene in said panel of genes with a predefined coefficient, and (2) combining the weighted expression to provide said test expression score, wherein said test genes are weighted to contribute at least 25% to said test expression score; and either
(3)(a) diagnosing a patient in whose sample said test expression score exceeds a first reference expression score as having an increased likelihood of disease recurrence or having an increased likelihood of chemotherapy response compared to a reference population; or
(3)(b) diagnosing a patient in whose sample said test expression score does not exceed a second reference expression score as not having an increased likelihood of disease recurrence or not having an increased likelihood of chemotherapy response compared to a reference population.

### Embodiment 2

The method of Embodiment 1, wherein said test genes are weighted to contribute at least 30%, 40%, 50%, 60%, 70%, 80%, 90%, 95%, 96%, 97%, 98%, 99%, or 100% of the total weight given to the expression of all of said panel of genes in said test expression score.

### Embodiment 3

The method of Embodiment 1 or Embodiment 2, wherein said panel of genes comprises at least 4, 5, 6, 7, 8, 9, 10, 12, 14, 16, 18, 20, 22, 24, 26, 28, 30, 32, or 34 test genes selected from Table 40, wherein at least 2, 3, 4, 5, 6, 7, 8, 9, 10, 12, 14, 16, 18, 20, or 22 of said test genes are CCP genes listed in Table 40 and at least 1, 2, 3, 4, 5, 6, or 7 of said test genes is an immune gene listed in Table 40.

### Embodiment 4

The method of any one of Embodiments 1 to 3, wherein said test genes comprise at least gene numbers 1 through 30 of Table 40.

### Embodiment 5

The method of any one of Embodiments 1 to 4, wherein said test genes comprise at least gene numbers 1 through 31 of Table 40.

### Embodiment 6

The method of any one of Embodiments 1 to 5, wherein said test genes comprise the genes listed in Table 40.

### Embodiment 7

The method of any one of Embodiments 1 to 6, wherein said test genes further comprise at least one of gene numbers 31 through 34 in Table 40.

### Embodiment 8

The method of Embodiment 7, wherein said test genes further comprise ABCC5.

### Embodiment 9

The method of any one of Embodiments 1 to 8, wherein said measuring step comprises:
measuring the amount of panel mRNA in said sample transcribed from each of between 3 and 500 panel genes, or measuring the amount of cDNA reverse transcribed from said panel mRNA; and
measuring the amount of housekeeping mRNA in said sample transcribed from one or more housekeeping genes, or measuring the amount of cDNA reverse transcribed from said housekeeping mRNA.

### Embodiment 10

The method of any one of Embodiments 1 to 9, wherein said first and second reference expression scores are the same.

### Embodiment 11

The method of any one of Embodiments 1 to 10, wherein half of breast cancer patients in said reference population have an expression score exceeding said first reference expression score and half of breast cancer patients in said reference population have an expression score not exceeding said first reference expression score.

### Embodiment 12

The method of any one of Embodiments 1 to 11, wherein one third of breast cancer patients in said reference population have an expression score exceeding said first reference expression score and one third of breast cancer patients in said reference population have an expression score not exceeding said second reference expression score.

### Embodiment 13

The method of Embodiment 12, comprising (a) diagnosing a patient in whose sample said test expression score exceeds said first reference expression score as having an increased likelihood of disease recurrence or having an increased likelihood of chemotherapy response compared to said reference population; (b) diagnosing a patient in whose sample said test expression score does not exceed said second reference expression score as having an increased likelihood of disease recurrence or having an increased likelihood of chemotherapy response compared to said reference population; or (c) diagnosing a patient in whose sample said test expression score exceeds said second reference expression score but does not exceed said first reference expression score as having no increased likelihood of disease recurrence or having no increased likelihood of chemotherapy response compared to said reference population.

### Embodiment 14

The method of any one of Embodiments 1 to 13, wherein disease recurrence is chosen from the group consisting of distant metastasis of the primary breast cancer; local metastasis of the primary breast cancer; recurrence of the primary breast cancer; progression of the primary breast cancer; and development of locally advanced, metastatic disease.

### Embodiment 15

The method of any one of Embodiments 1 to 14, wherein chemotherapy response is pathological complete response.

### Embodiment 16

A method for determining a breast cancer test patient's likelihood of breast cancer recurrence, comprising:
(1) measuring, in a sample obtained from said test patient, the expression levels of a panel of genes comprising at least 3 test genes selected from Table 40, wherein at least two of said test genes are CCP genes listed in Table 40 and at least one of said test genes is an immune gene listed in Table 40;
(2) providing a test expression score by (1) weighting the determined expression of each gene in said panel of genes with a predefined coefficient, and (2) combining the weighted expression to provide said test expression score, wherein said test genes are weighted to contribute at least 25% to said test expression score; and
(3) diagnosing said test patient as having either (a) an increased likelihood of disease recurrence based at least in part on said test expression score exceeding a first reference expression score or (b) no increased likelihood of disease recurrence based at least in part on said test expression score not exceeding a second reference expression score.

### Embodiment 17

The method of Embodiment 16, wherein said test genes are weighted to contribute at least 30%, 40%, 50%, 60%, 70%, 80%, 90%, 95%, 96%, 97%, 98%, 99%, or 100% of the total weight given to the expression of all of said panel of genes in said test expression score.

### Embodiment 18

The method of any one of Embodiments 16 or 17, wherein said panel of genes comprises at least 4, 5, 6, 7, 8, 9, 10, 12, 14, 16, 18, 20, 22, 24, 26, 28, 30, 32, or 34 test genes selected from Table 40, wherein at least 2, 3, 4, 5, 6, 7, 8, 9, 10, 12, 14, 16, 18, 20, or 22 of said test genes are CCP genes listed in Table 40 and at least 1, 2, 3, 4, 5, 6, or 7 of said test genes is an immune gene listed in Table 40.

### Embodiment 19

The method of any one of Embodiments 16 to 18, wherein said test genes comprise at least gene numbers 1 through 30 of Table 40.

### Embodiment 20

The method of any one of Embodiments 16 to 19, wherein said test genes comprise at least gene numbers 1 through 31 of Table 40.

### Embodiment 21

The method of one of Embodiments 16 to 20, wherein said test genes comprise the genes listed in Table 40.

### Embodiment 22

The method of any one of Embodiments 16 to 21, wherein said test genes further comprise at least one of gene numbers 31 through 34 in Table 40.

### Embodiment 23

The method of Embodiment 22, wherein said test genes further comprise ABCC5.

### Embodiment 24

The method of any one of Embodiments 16 to 23, wherein said measuring step comprises:
measuring the amount of panel mRNA in said sample transcribed from each of between 3 and 500 panel genes, or measuring the amount of cDNA reverse transcribed from said panel mRNA; and
measuring the amount of housekeeping mRNA in said sample transcribed from one or more housekeeping genes, or measuring the amount of cDNA reverse transcribed from said housekeeping mRNA.

### Embodiment 25

The method of any one of Embodiments 16 to 24, wherein said first and second reference expression scores are the same.

### Embodiment 26

The method of any one of Embodiments 16 to 25, wherein half of breast cancer patients in a reference population have an expression score exceeding said first reference expression score and half of breast cancer patients in said reference population have an expression score not exceeding said first reference expression score.

### Embodiment 27

The method of any one of Embodiments 16 to 26, wherein one third of breast cancer patients in a reference population have an expression score exceeding said first reference expression score and one third of breast cancer patients in said reference population have an expression score not exceeding said second reference expression score.

### Embodiment 28

The method of Embodiment 12, comprising diagnosing said test patient as having (a) an increased likelihood of disease recurrence if said test expression score exceeds said first reference expression score; (b) a decreased likelihood of disease recurrence if said test expression score does not exceed said second reference expression score; or (c) no increased likelihood of disease recurrence if said test expression score exceeds said second reference expression score but does not exceed said first reference expression score.

### Embodiment 29

The method of any one of Embodiments 16 to 28, wherein disease recurrence is chosen from the group consisting of distant metastasis of the primary breast cancer; local metastasis of the primary breast cancer; recurrence of the primary breast cancer; progression of the primary breast cancer; and development of locally advanced, metastatic disease.

### Embodiment 30

A method for determining a breast cancer patient's likelihood of breast cancer recurrence, comprising:
(1) measuring, in a sample obtained from said patient, the expression levels of a panel of genes comprising at least 3 test genes selected from Table 40, wherein at least two of said test genes are CCP genes listed in Table 40 and at least one of said test genes is an immune gene listed in Table 40;
(2) providing a test expression score by (1) weighting the determined expression of each gene in said panel of genes with a predefined coefficient, and (2) combining the weighted expression to provide said test expression score, wherein said test genes are weighted to contribute at least 25% to said test expression score;
(3) providing a test prognostic score combining said test expression score with at least one test clinical score representing at least one clinical variable; and
(4) diagnosing said patient as having either (a) an increased likelihood of breast cancer recurrence based at least in part on said test prognostic score exceeding a first reference prognostic score or (b) no increased likelihood of breast cancer recurrence based at least in part on said test prognostic score not exceeding a second reference prognostic.

### Embodiment 31

The method of Embodiment 30, wherein said at least one clinical score incorporates at least one clinical variable chosen from the group consisting of node status, tumor size and tumor grade.

### Embodiment 32

The method of any one of Embodiments 30 or 31, wherein said prognostic scores incorporate (a) a first clinical score representing node status and (b) a second clinical score representing tumor size.

### Embodiment 33

The method of Embodiment 32, wherein (a) a patient's node status is negative (N0) if said patient was found to have no positive lymph nodes and positive (N1) if said patient was found to have between one and three positive lymph nodes and/or (b) the value for said second clinical score is the size of the tumor in centimeters.

### Embodiment 34

The method of any one of Embodiments 30 to 33, wherein said prognostic scores are calculated according to a formula comprising the following terms: (D×Tumor Size)+(E×node status)+(B×CCP score)−(A×Immune score)+(C×ABCC5).

### Embodiment 35

The method of any one of Embodiments 30 to 33, wherein said prognostic scores are calculated according to a formula comprising the following terms: (D×Tumor Size [cm[)+(E×node status [0 or 1])+(B×CCP score)−(A×Immune score)+(C×ABCC5)−(F×PGR).

### Embodiment 36

The method of Embodiment 35, wherein said prognostic scores are calculated according to a formula comprising the following terms: (0.54×CCP score)−(0.44×Immune score)+(0.40×ABCC5)−(0.09×PGR)+(0.48×Tumor Size [cm])+(0.73×node status [0 or 1]).

### EXAMPLES

### Example 1

The following example describes identification of the immune system genes (ISGs) and other cancer prognostic genes (OCPGs) that can be used for the prognosis of cancer.

Description of Data. Seven public breast cancer datasets (GEO accession numbers GSE2034 (Yixin Wang et al. The Lancet, 365(9460):671-679, February 2005), GSE6532 (Sherene Loi et al. BMC Genomics, 9(1):239+, May 2008), GSE7390 (Christine Desmedt et al. Clinical Cancer Research: an official journal of the American Association for Cancer Research, 13(11):3207-3214, June 2007), and GSE9195 (Sherene Loi et al. BMC Genomics, 9(1):239+, May 2008), GSE11121 (M. Schmidt et al. Cancer Research, 68(13):5405-5413, July 2008), GSE12093 (Yi Zhang et al. Breast Cancer Research and Treatment, 116(2):303-309, July 2009), and GSE17705 (W. Fraser Symmans et al. Journal of Clinical Oncology, 28(27):4111-4119, September 2010]) in which the patients were not treated with chemotherapy and the samples were run on Affymetrix arrays. ER, lymph node, and tamoxifen treatment statuses were available for the majority of patients. Table 27 gives a breakdown of the patients' clinical data. Distant metastasis-free survival (DMFS), was calculated as the time in years from surgery to distant metastasis. Data was not available to calculate DMFS for 30 of the 1609 total patients. DMFS was censored for patients that were lost to follow-up before distant metastasis or that experienced distant metastasis after 10 years. Using this definition, 376 (24%) distant metastases were observed.

TABLE 27
Summary of Patient Characteristics

| | | | | Lymph Node | | | | | |
|---|---|---|---|---|---|---|---|---|---|
| | ER Status | | | Status | | | Tamoxifen Status | | |
| Dataset | + | − | ? | + | − | ? | + | − | All |
| GSE2034 | 208 | 78 | 0 | 0 | 286 | 0 | 0 | 286 | 286 |
| GSE6532 | 349 | 45 | 20 | 143 | 250 | 21 | 277 | 137 | 414 |
| GSE7390 | 134 | 64 | 0 | 0 | 198 | 0 | 0 | 198 | 198 |
| GSE9195 | 77 | 0 | 0 | 36 | 41 | 0 | 77 | 0 | 77 |
| GSE11121 | 0 | 0 | 200 | 0 | 200 | 0 | 0 | 200 | 200 |
| GSE12093 | 136 | 0 | 0 | 0 | 136 | 0 | 136 | 0 | 136 |
| GSE17705 | 298 | 0 | 0 | 112 | 175 | 11 | 298 | 0 | 298 |
| Total | 1202 | 187 | 220 | 291 | 1286 | 32 | 788 | 821 | 1609 |

RNA Expression by Microarray

All samples were run on either the Affymetrix Human Genome U133A or Human Genome U133 Plus 2.0 micro arrays. This analysis considers more than 22,000 probes in common between these two arrays. The arrays were pre-processed separately for each dataset. A cell-cycle progression (CCP) score was calculated as the average expression of a large group of probes known to be cell-cycle genes.

Missing ER Status

There were 20 patients from the GSE6532 dataset missing ER status. There are two clear groups for the patients with unknown status. The 10 patients in the low ESR1 group were considered ER- and the 10 in the high ESR1 group were considered ER+. None of the patients from the GSE11121 dataset had ER status. The 42 tumors with ESR1 expression less than 9.5 were considered ER and the 158 tumors with ESR1 expression greater than 9.5 were considered ER+. The remainder of this Example focuses on the 1343 ER+ patients with known lymph node status.

A random effects meta-analysis was carried out to assess the ability of the CCP score to predict DMFS in the ER+ samples across all datasets. GSE6532 was the only dataset with both patients that were and were not treated with Tamoxifen. As a result, GSE6532 was treated as two datasets: one consisting of treated individuals and the other consisting of untreated individuals. For each dataset the effect of CCP on DMFS was calculated from a Cox proportional hazards regression model that accounted for lymph node status. A summary effect and p-value were calculated by weighting each dataset's estimated effect by the inverse of its variance. Variance due to heterogeneity of the estimates was accounted for. The summary DMFS hazard ratio for CCP was 3.63 (95% Cl 2.78, 4.74). The corresponding p-value was 3.5e-21.

Some Preferred Predictors of DMFS after Accounting for CCP

Summary HRs and p-values were calculated for all probes using a similar method as with CCP. The only difference was, in addition to lymph node status, CCP was accounted for in the Cox models. There were 55 probes with p-values less than 0.00001. Hierarchical clustering of the expression of the 100 most significant probes from the meta-analysis was performed in the GSE2034 dataset. Ward's method, which minimizes the within cluster variance, was the criterion for clustering. The distance between each pair of samples was calculated as one minus the absolute value of Spearman's correlation coefficient between the samples. A dendrogram of the resulting clusters yielded two major clusters for the 100 top probes (i.e., 100 most significant probes).

There are two main clusters of probes. The probes in one cluster (Table 5) do not seem to represent a clearly defined set of genes or pathway and are referred to as other cancer prognostic genes or OCPGs; whereas, the other cluster has mostly probes that related to immune genes (Immune System Genes or ISGs). Notably, higher expression of the ISGs was correlated with better prognosis. In the OCPG group there were genes where higher expression correlated with better prognosis (bpOCPGs) and genes where higher expression was associated with worse prognosis (wpOCPGs). Within the cluster of immune related probes there are three smaller clusters: a cluster of probes whose genes are associated with B-cells (Table 3) (BCRGs), a cluster of probes whose genes are associated with T-cells (Table 4) (TCRGs), and a cluster probes whose genes are associated with HLA class II activation (Table 5) (HLAGs).

The average pairwise-correlations between the probes in each of the three clusters were 0.83, 0.59, and 0.74 for the B-cell, T-cell, and HLA class II activation clusters, respectively. The average expression across the probes in each group was calculated. A curvilinear relationship between the T-cell and HLA class II activation cluster averages was found. This observation is consistent with a majority of the T-cell group of probes hitting the lower limit of detection of the microarrays.

A series of meta-analyses were carried out to assess the ability of the 3 immune cluster gene set (B-cell, T-cell, and HLA class II activation cluster) averages to predict DMFS. For each of the immune cluster averages, a meta-analysis was performed by including lymph node status, CCP average, and the immune cluster average. Then lymph node status, CCP average, and each pair of immune cluster averages were included in meta-analysis models. Finally, lymph node status, CCP average, and all three immune cluster averages were included. Summary HRs and p-values for the meta-analyses were calculated and can be found in Table 28. A number of genes identified in this study were further examined in a set of commercially available breast cancer tumor samples by quantitative PCR in the following examples.

TABLE 28
Summary of Meta-Analysis Data

| | | HR | p-value |
|---|---|---|---|
| B-cell Cluster | Lymph Node Status | 0.81 (0.74, 0.88) | 4.5e−07 |
| Average | CCP Average | | |
| B-cell Cluster | Lymph Node Status | 0.93 (0.81, 1.06) | 0.26 |
| Average | CCP Average | | |
| | T-cell Cluster Average | | |
| B-cell Cluster | Lymph Node Status | 0.88 (0.79, 0.99) | 0.032 |
| Average | CCP Average | | |
| | HLA Class II Activation | | |
| | Average | | |
| T-cell Cluster | Lymph Node Status | 0.55 (0.44, 0.69) | 1.3e−07 |
| Average | CCP Average | | |
| T-cell Cluster | Lymph Node Status | 0.63 (0.45, 0.89) | 0.0092 |
| Average | CCP Average | | |
| | T-cell Cluster Average | | |
| T-cell Cluster | Lymph Node Status | 0.66 (0.46, 0.93) | 0.018 |
| Average | CCP Average | | |
| | HLA Class II Activation | | |
| | Average | | |
| HLA Class II | Lymph Node Status | 0.66 (0.57, 0.77) | 6.4e−08 |
| Activation | CCP Average | | |
| Average | | | |
| HLA Class II | Lymph Node Status | 0.75 (0.61, 0.94) | 0.011 |
| Activation | CCP Average | | |
| Average | T-cell Cluster Average | | |
| HLA Class II | Lymph Node Status | 0.84 (0.66, 1.07) | 0.15 |
| Activation | CCP Average | | |
| Average | B-cell Cluster Average | | |

### Example 2

Based on the results from a meta-analysis involving 7 breast cancer microarray datasets as described in Example 1, 32 qPCR assays (Table 29) were selected for further testing. These assays, together with 15 assays for housekeeper genes, were included on the Immunity Panel 1 TLDA card and run, in duplicate, against 47 ER+ breast cancer samples purchased from ProteoGenex.

TABLE 29
Genes and Assays IDs used for qPCR studies

| Gene Abbreviation | Gene Assay ID |
|---|---|
| CCL19 | Hs00171149_m1 |
| CCL5 | Hs00174575_m1 |
| CCR2 | Hs00174150_m1 |
| CD38 | Hs01120071_m1 |
| CD74 | Hs00269961_m1 |
| CEP57 | Hs00206534_m1 |
| CXCL12 | Hs00171022_m1 |
| EVI2B | Hs00272421_s1 |
| EVI2B | Hs00366769_m1 |
| HCLS1 | Hs00945386_m1 |
| HLA-DMA | Hs00185435_m1 |
| HLA-DPA1 | Hs01072899_m1 |
| HLA-DPB1 | Hs00157955_m1 |
| HLA-DRA | Hs00219575_m1 |
| HLA-DRB1 | Hs99999917_m1 |
| HLA-E | Hs03045171_m1 |
| IGHM | Hs00378435_m1 |
| IGJ | Hs00376160_m1 |
| IGJ | Hs00950678_g1 |
| IGLL5/CKAP2 | Hs00382306_m1 |
| IRF1 | Hs00971965_m1 |
| IRF1 | Hs00971966_g1 |
| IRF4 | Hs00180031_m1 |
| ITGB2 | Hs01051739_m1 |
| LITAF | Hs01556091_m1 |
| NTM | Hs00275411_m1 |
| PECAM1 | Hs00169777_m1 |
| PTPN22 | Hs00249262_m1 |
| PTPRC | Hs00894732_m1 |
| SELL | Hs01046459_m1 |
| TRDV3/TRDV1 | Hs00379146_m1 |
| ZFP36L2 | Hs00272828_m1 |

qPCR Data Quality

For each replicate of each sample, ΔCT was calculated by subtracting the average CT of the housekeeper gene from the CT of each of the genes of interest. Duplicate ΔCT values were averaged. Summarized ACTs were not calculated for samples missing any housekeeper gene CTs, for duplicate ΔCT values whose standard deviation exceeded 3, or for incomplete duplicates. Seven samples were excluded from further analysis because they were missing DCT for 9 or more assays. The genes IGJ, IRF1, and EVI2B were represented by two probes each. The two probes for IGJ were well correlated and neither was missing any values. The same was true for the two assays for IRF1. Consequently, the averages of the redundant assays were used in place of the individual measurements. The two assays for EVI2B were not as well correlated. Assay Hs00366769_m1 shows very low expression for a couple of samples compared to Hs00272421_s1 and is missing ΔCT altogether for another sample where the expression was quite high (−ΔCT=−1:97) for Hs00272421_s1. This may be an indication that some patients are missing the transcript that is queried by Hs00366769_m1. The assay for HLA-DRB1 also demonstrates interesting behavior. The distribution has a very large range and is clearly multi-modal. Additionally, the assay produces missing values for 22 of the 39 samples. The assay for CCR2 was missing values for 21 of the 39 samples.

Immune Gene Clustering

Of the 29 unique genes of interest represented on the Immunity Panel 1 TLDA card 24 are genes related to the body's immune response. The immune genes were clustered based on their expression in the 39 good quality samples. Ward's method, which minimizes the within cluster variance, was the criterion for clustering. The distance between each pair of samples was calculated as one minus the absolute value of Spearman's correlation coefficient between the samples. The resulting dendrogram gave two clear clusters of genes (one of which is summarized Table 30 and the other in Table 31). The averages of the genes in each cluster and the correlation between each gene and the cluster averages were calculated. HLA-DRB1, CCR2, and Hs00366769_m1 for EVI2B were left out of the cluster averages due to their odd behavior (HLA-DRB1 and EVI2B) and missing values (HLA-DRB1 and CCR2). The correlation between each of the genes and the average of cluster 1 is shown in Table 30. The correlation between each of the genes and the average of cluster 2 is shown in Table 31.

TABLE 30
Genes in Cluster 1 and Correlation with Their Average

| Gene | Gene | | Cluster in |
|---|---|---|---|
| # | Symbol | Correlation | Public Data |
| 1 | IRF4 | 0.9 | T-Cell |
| 2 | CCL19 | 0.85 | T-Cell |
| 3 | SELL | 0.82 | T-Cell |
| 4 | CD38 | 0.81 | T-Cell |
| 5 | CCL5 | 0.78 | T-Cell |
| 6 | IGLL5/CKAP2 | 0.78 | B-Cell |
| 7 | CCR2 | 0.77 | T-Cell |
| 8 | TRDV3/TRDV1 | 0.76 | T-Cell |
| 9 | IGHM | 0.76 | B-Cell |
| 10 | IGJ | 0.74 | B-Cell |
| 11 | PTPRC | 0.72 | HLA Activation |

TABLE 31
Cluster 2Genes and Correlation with Average

| Gene | Gene | | Cluster in |
|---|---|---|---|
| # | Symbol | Correlation | Public Data |
| 1 | ITGB2 | 0.8 | HLA Activation |
| 2 | EVI2B | 0.8 | HLA Activation |
| 3 | HCLS1 | 0.8 | HLA Activation |
| 4 | HLA-DPB1 | 0.76 | HLA Activation |
| 5 | HLA-E | 0.75 | T-Cell |
| 6 | HLA-DPA1 | 0.73 | HLA Activation |
| 7 | HLA-DRA | 0.69 | HLA Activation |
| 8 | HLA-DMA | 0.67 | HLA Activation |
| 9 | PECAM1 | 0.65 | HLA Activation |
| 10 | EVI2B | 0.62 | HLA Activation |
| 11 | PTPN22 | 0.56 | T-Cell |
| 12 | IRF1 | 0.54 | T-Cell |
| 13 | CD74 | 0.42 | HLA Activation |
| 14 | HLA-DRB1 | −0.25 | HLA Activation |

The only gene that was a member of cluster 1 that was not a member of the B-cell or T-cell cluster in the public datasets was PTPRC; however, it also had the lowest correlation with the cluster 1 average of all the genes used to calculate the average. Only HLA-E belonged to a cluster other than the HLA activation cluster in the public datasets but had a correlation greater than 0.60 with the cluster 2 average in this dataset. The Hs00366769_m1 probe for EVI2B had worse correlation with the HLA activation cluster than the Hs00272421_s1 assay. The cluster 1 average has a much wider range than cluster 2 average and their correlation is moderate.

The assay for HLA-DRB1 and the Hs00366769_m1 assay for EVI2B show evidence of copy number differences for some samples. The assay for CCR2 has low expression and is missing many values. Accordingly, these assays, in some panels and aspects of the disclosure are not included. A few other assays do not correlate well with the other immune genes. Otherwise the quality of the rest of the assays appears to be high.

### Example 3

This experiment was run to determine an exemplary group of assays for breast cancer prognosis using qPCR.

A panel (e.g., using a TLDA card) was designed to measure CCP score, ABCC5 expression, and the expression of three hormone receptors ESR1, ERBB2, and PGR. This version of the CCP has 14 housekeeper genes 24 CCP genes, and two assays for each of the other genes. It was run on the Nottingham pilot and the assays performed well. The other TLDA card of interest is Immunity Panel 2. The Immunity Panel 2 is similar to the Immunity Panel 1 TLDA card except five housekeeper genes with long amplicons (MMADHC, RPL37, RPL38, RPL4, and UBA52), two genes with possible copy number changes (EVI2B and HLA-DRB1), one gene with low expression (CCR2), and one gene that did not correlate with other immune genes (CD74) were replaced with two assays for CALD1, two assays for HLA-DRB1/3, and one assay for each of DUSP4, PDGFB, RACGAP1, SLC4A8, and SLC35E3.

Experimental Design

Both the CCP Panel for breast cancer and Immunity Panel 2 TLDA cards were run in duplicate against 71 ER+ breast cancer samples purchased from ProteoGenex.

### CCP Breast Cancer TLDA Card

Passing quality CCP scores were calculated for 68 of the 71 samples. The relationship between each of the CCP genes and the CCP score was determined. Relationships between the two probes that measure the expression of each of ABCC5, ERBB2, ESR1, and PGR were also determined.

Immunity Panel 2 TLDA Card

For each replicate of each sample, ΔCT was calculated by subtracting the average CT of the housekeeper gene from the CT of each of the genes of interest. Duplicate ΔCT values were averaged. Summarized ACTs were not calculated for samples missing any housekeeper gene CTs, for duplicate ΔCT values whose standard deviation exceeded 3, or for incomplete duplicates. Five samples were excluded from further analysis because they were missing ΔCT for 12 or more assays. The genes IGJ, IRF1, CALD1, and HLA-DRB1/3 were represented by two probes each. The two probes for IGJ and are well correlated and neither were missing any values. The same was true for the two assays for IRF1. Consequently, the averages of the redundant assays were used in place of the individual measurements. The two assays for CALD1 are poorly correlated. Assay Hs00921982 m1 has a wider range of expression, higher expression, and more missing values compared to Hs00263998 m1. Both assays for HLA-DRB1/3 also demonstrated interesting behavior. Both assays have a very large range and are multi-modal. Assay Hs00734212 m1 is missing 10 values, while assay Hs02339733 m1 is missing 24. The probe for RACGAP1 did not appear to work as it was missing 62 values.

Immune Gene Clustering

Of the 34 unique genes of interest represented on the Immunity Panel 1 TLDA card 23 are genes related to immune response in human. The immune genes were clustered based on their expression in the 66 good quality samples. Ward's method, which minimizes the within cluster variance, was the criterion for clustering. The distance between each pair of samples was calculated as one minus the absolute value of Spearman's correlation coefficient between the samples. A dendrogram generated from this analysis revealed two clear clusters of genes: one cluster is in Table 32 and the other cluster is in Table 33. The averages of the genes in each cluster and the correlation between each gene and the cluster averages were calculated. Both probes for HLA-DRB1/3 were left out of the cluster averages due to their odd behavior. The correlation between each of the genes and the average of cluster 1 is shown in Table 32. The correlation between each of the genes and the average of cluster 2 is shown in Table 33.

TABLE 32
Cluster 1 Genes and the Correlation with Their Average

| Gene | Gene | | Cluster in |
|---|---|---|---|
| # | Symbol | Correlation | Public Data |
| 1 | IRF4 | 0.95 | T-Cell |
| 2 | CD38 | 0.91 | T-Cell |
| 3 | SELL | 0.89 | T-Cell |
| 4 | CCL5 | 0.89 | T-Cell |
| 5 | IGHM | 0.88 | B-Cell |
| 6 | IGLL5/CKAP2 | 0.84 | B-Cell |
| 7 | PTPRC | 0.81 | HLA Activation |
| 8 | IGJ | 0.79 | B-Cell |
| 9 | IRF1 | 0.78 | T-Cell |
| 10 | EVI2B | 0.78 | HLA Activation |
| 11 | CCL19 | 0.77 | T-Cell |
| 12 | TRDV3/TRDV1 | 0.76 | T-Cell |
| 13 | PTPN22 | 0.74 | T-Cell |
| 14 | PECAM1 | 0.57 | HLA Activation |

TABLE 33
Cluster 2 Genes and the Correlation with Their Average

| Gene | Gene | | Cluster in |
|---|---|---|---|
| # | Symbol | Correlation | Public Data |
| 1 | HLA-DMA | 0.92 | HLA Activation |
| 2 | HLA-DPB1 | 0.91 | HLA Activation |
| 3 | HLA-DRA | 0.89 | HLA Activation |
| 4 | HLA-E | 0.88 | T-Cell |
| 5 | HLA-DPA1 | 0.87 | HLA Activation |
| 6 | HCLS1 | 0.85 | HLA Activation |
| 7 | ITGB2 | 0.82 | HLA Activation |
| 8 | HLA-DRB3 | 0.56 | HLA Activation |
| 9 | HLA-DRB3/HLA-DRB1 | 0.47 | HLA Activation |

The immune genes clustered similarly to how they clustered the first time they were run on commercial samples with a few exceptions. Specifically, EVI2B, IRF1, PECAM1, and emph-PTPN22 clustered with the other set of genes. All of these genes except EVI2B had some of the lowest correlations with the cluster average in the last run. They were also among the lowest correlations in this dataset; although, their correlations with the cluster 1 average are higher than their correlations with the cluster 2 average in the last set of samples. The cluster 1 average has a much wider range than cluster 2 average and their correlation is moderate.

Relationships between CCP score and immune gene cluster 1 and 2 averages were determined. The assay for RACGAP1, both assays for HLA-DRB1/3, and assay Hs00921982 m1 for CALD1 in some aspects and panels of the disclosure are not included. CCP score and the immune cluster averages are uncorrelated.

### Example 4

This study initially involved 537 breast cancer patients. All patients were ER+ and node negative. For each patient, dates were recorded for the following events: surgery; Tamoxifen start and end; breast, axillary, sub-clavicular fossa, and distant metastatic relapse; loss to follow-up; and death. The cause of death and disease status at death were also included.

The primary outcome of interest, distant metastasis-free survival (DMFS), was calculated as the time in years from surgery to distant metastasis. DMFS was censored for patients that were lost to follow-up before experiencing distant metastasis or that experienced distant metastasis after 10 years. Using this definition, 63 distant metastasis events were observed.

Other clinical data for each patient included age (mean=56.6, sd=10.6) and type of adjuvant therapy status (414 tamoxifen, 39 hormone therapy other than tamoxifen, and 84 none). Information on each tumor included ER and PR status (both on a scale from 0 to 8), size (mm), histologic type, and grade (148 poorly differentiated, 255 moderately differentiated, 133 well differentiated, and 1 missing). Patients that received tamoxifen or another hormone therapy were treated the same throughout the analysis.

qPCR Data
qPCR Assay Details and CCP Score

The CCP score was calculated from RNA expression of 23 CCP genes (Panel O) normalized by 9 housekeeper genes (HK). The relative numbers of CCP genes and HK genes were optimized in order to minimize the variance of the CCP score. The CCP score is the unweighted mean of C_{T} values for CCP gene expression, normalized by the unweighted mean of the HK genes so that higher values indicate higher expression. One unit is equivalent to a two-fold change in expression. The CCP scores were centered by the mean value, again determined in the training set.

A dilution experiment was performed on four of the commercial prostate samples to estimate the measurement error of the CCP score (se=0.10) and the effect of missing values. It was found that the CCP score remained stable as concentration decreased to the point of 10 failures out of the total 24 CCP genes. Based on this result, samples with more than 9 missing values were not assigned a CCP score.

From each FFPE sample block one 5 μm section was cut and stained with haematoxylin and eosin. Tumor areas were marked by a pathologist. Additional two 10 μm sections were cut directly adjacent to the H&E stained section. Tumor areas on the unstained sections were identified by alignment with the marked areas on the H&E stain and macro-dissected manually into Eppendorff tubes. Sections were deparaffinized by xylene extractions followed by washes with ethanol. After an overnight incubation with proteinase K, deparaffinized tissue was subjected to RNA extraction using the Qiagen miRNAeasy kit according to manufacturer's instructions. Total RNA was treated with DNASE I to remove potential genomic DNA contamination. Final RNA yield was determined on a Nanodrop spectrophotometer.

For each sample 500 ng RNA was converted to cDNA using the high capacity cDNA archive kit (Applied Biosystems). Newly synthesized cDNA served as template for replicate pre-amplification reactions. Each of the reactions contained 3 μl cDNA and a pool of Taqman™ assays for all 38 genes in the signature (14 housekeeping genes, 24 cell cycle genes). Preamplification was run for 14 cycles to generate sufficient total copies even from a low copy sample to inoculate individual PCR reactions for 38 genes. Preamplification reactions were diluted 1:20 before loading on Taqman™ low density arrays (TLDA, Applied Biosystems). Raw data for the calculation of the CCP score were the Cₜ values of the 46 genes from the TLDA arrays. The CCP score was the unweighted mean of Cₜ values for cell cycle gene expression, normalized by the unweighted mean of the house keeper genes so that higher values indicate higher expression. One unit is equivalent to a two-fold change in expression. The CCP scores were centered by the mean value determined in the commercial training set.

CCP scores were unusable for 36 samples: 21 for too many missing housekeeper genes (12 were required), 14 for too many missing CCP genes (18 were required), and 1 because the standard deviation of the by-card CCP scores was greater than 0.5. Therefore, 498 (93%) samples received passing CCP scores.

Other qPCR Expression

In addition to the CCP genes, ABCC5, PGR and ESR1 were also measured via the same process described above. Two assays were selected to measure the expression of each of ABCC5 (Assay ID nos. Hs00981085_m1 and Hs00981087_m1) and PGR (Assay ID nos. Hs01556702_m1 and Hs01556707_m1). The expression for the two assays was averaged and 513 patients had acceptable values.

These samples were combined with 181 additional samples from patients with positive nodes. This combined cohort was analyzed as described above with the following distinction and as further noted below: Use of hormone therapy as a time dependent covariate was introduced.

TABLE 34
Genes of Panel O Ranked by Correlation to CCP Mean

| Gene | | | Correlation to |
|---|---|---|---|
| # | Gene | Assay | CCP Mean |
| 1 | ASPM | Hs00411505_m1 | 0.89 |
| 2 | MCM10 | Hs00960349_m1 | 0.89 |
| 3 | BUB1B | Hs01084828_m1 | 0.88 |
| 4 | KIF20A | Hs00993573_m1 | 0.88 |
| 5 | SKA1 | Hs00536843_m1 | 0.88 |
| 6 | CDKN3 | Hs00193192_m1 | 0.87 |
| 7 | PRC1 | Hs00187740_m1 | 0.87 |
| 8 | RAD54L | Hs00269177_m1 | 0.87 |
| 9 | RRM2 | Hs00357247_g1 | 0.87 |
| 10 | PTTG1 | Hs00851754_u1 | 0.86 |
| 11 | NUSAP1 | Hs01006195_m1 | 0.85 |
| 12 | RAD51 | Hs00153418_m1 | 0.84 |
| 13 | CDK1 | Hs00364293_m1 | 0.83 |
| 14 | KIAA0101 | Hs00207134_m1 | 0.81 |
| 15 | KIF11 | Hs00189698_m1 | 0.81 |
| 16 | PBK | Hs00218544_m1 | 0.81 |
| 17 | CDCA3 | Hs00229905_m1 | 0.78 |
| 18 | CENPF | Hs00193201_m1 | 0.78 |
| 19 | DTL | Hs00978565_m1 | 0.77 |
| 20 | TK1 | Hs01062125_m1 | 0.76 |
| 21 | ASF1B | Hs00216780_m1 | 0.74 |
| 22 | PLK1 | Hs00153444_m1 | 0.7 |
| 23 | CENPM | Hs00608780_m1 | 0.66 |

TABLE 35
CCP Genes Ranked by Univariate P-Value

| Gene | Gene | | Univariate |
|---|---|---|---|
| # | Symbol | Assay ID | p-value |
| 1 | CDKN3 | Hs00193192_m1 | 1.00E−08 |
| 2 | SKA1 | Hs00536843_m1 | 2.30E−07 |
| 3 | BUB1B | Hs01084828_m1 | 3.50E−07 |
| 4 | KIF20A | Hs00993573_m1 | 7.10E−07 |
| 5 | RRM2 | Hs00357247_g1 | 9.00E−07 |
| 6 | ASPM | Hs00411505_m1 | 2.70E−06 |
| 7 | NUSAP1 | Hs01006195_m1 | 4.60E−06 |
| 8 | DTL | Hs00978565_m1 | 9.50E−06 |
| 9 | PLK1 | Hs00153444_m1 | 1.20E−05 |
| 10 | CDK1 | Hs00364293_m1 | 1.60E−05 |
| 11 | PRC1 | Hs00187740_m1 | 2.30E−05 |
| 12 | PTTG1 | Hs00851754_u1 | 2.30E−05 |
| 13 | MCM10 | Hs00960349_m1 | 3.60E−05 |
| 14 | CENPM | Hs00608780_m1 | 7.90E−05 |
| 15 | CENPF | Hs00193201_m1 | 1.30E−04 |
| 16 | KIF11 | Hs00189698_m1 | 2.50E−04 |
| 17 | RAD51 | Hs00153418_m1 | 8.00E−04 |
| 18 | PBK | Hs00218544_m1 | 8.70E−04 |
| 19 | TK1 | Hs01062125_m1 | 1.00E−03 |
| 20 | RAD54L | Hs00269177_m1 | 2.00E−03 |
| 21 | CDCA3 | Hs00229905_m1 | 3.70E−03 |
| 22 | KIAA0101 | Hs00207134_m1 | 1.90E−02 |
| 23 | ASF1B | Hs00216780_m1 | 4.40E−02 |

TABLE 36
Housekeeper Genes

| Gene Symbol | Assay ID |
|---|---|
| CLTC | Hs00191535_m1 |
| PPP2CA | Hs00427259_m1 |
| PSMA1 | Hs00267631_m1 |
| PSMC1 | Hs02386942_g1 |
| RPL13A (RPL13AP5) | Hs03043885_g1 |
| RPL8 | Hs00361285_g1 |
| RPS29 | Hs03004310_g1 |
| SLC25A3 | Hs00358082_m1 |
| TXNL1 | Hs00355488_m1 |

As previously described for the node-negative samples, gene expression data was collected for the new node-positive samples and CCP scores and average ABCC5 and PGR expression were calculated. CCP scores were considered acceptable if at least 17 CCP genes were adequately measured and the standard deviation of the replicate CCP scores was less than 0.5. Both assays for ABBC5 were required to yield quality values while only one of the two PGR assays was considered sufficient. After removing samples that did not meet the quality requirements, 595 patients from the combined cohort remained. The correlation with the CCP score as well as the p-value from univariate analysis of DMFS for each CCP gene is given in Tables 34 & 35.

Hormone therapy was included as a time dependent covariate instead of a binary indicator of treatment. The effect of hormone therapy was only estimated in recipients during the time while it was being administered. When the exact dates of the beginning and end of therapy were unknown it was assumed that the patient received hormone therapy for the first five years after surgery (which is the standard of care).

Univariate analysis of DMFS and clinical and molecular variables was conducted on 565 patients with complete clinical and molecular data using Cox proportional hazards regression. The results are summarized in Table 37.

TABLE 37
Univariate Results

| Variable | p-value | HR (95% CI) |
|---|---|---|
| Age | 0.87 | 1 (0.98, 1.02) |
| Grade | 6.49E−05 | 1.84 (1.36, 2.5) |
| Tumor Size (cm) | 2.71E−06 | 2.07 (1.56, 2.74) |
| Node Positive | 5.05E−05 | 2.61 (1.68, 4.06) |
| CCP Score | 1.34E−07 | 1.91 (1.5, 2.44) |
| ABCC5 Expression | 1.53E−03 | 1.51 (1.17, 1.95) |
| PGR Expression | 0.07 | 0.92 (0.84, 1.01) |
| Hormone Therapy | 0.77 | 1.1 (0.6, 2.02) |

While neither hormone therapy nor PGR expression is significant in univariate analysis in this cohort, their interaction is highly predictive of DMFS (p-value=0.00016). In the interaction, the HR for hormone therapy when PGR is zero is 1.12 (0.59, 2.12), the HR for PGR while patients are untreated is 1.11 (0.96, 1.27), and the HR for PGR during treatment is 0.71 (0.59, 0.85).

Grade, tumor size, node status, CCP score, ABCC5 expression, and the interaction between PGR and hormone therapy were included together in a Cox model. Summarized results are in Table 38.

TABLE 38
Multivariate Results

| Variable | p-value | HR (95% CI) |
|---|---|---|
| Age | 0.87 | 1 (0.98, 1.02) |
| Grade | 6.49E−05 | 1.84 (1.36, 2.5) |
| Tumor Size (cm) | 2.71E−06 | 2.07 (1.56, 2.74) |
| Node Positive | 5.05E−05 | 2.61 (1.68, 4.06) |
| CCP Score | 1.34E−07 | 1.91 (1.5, 2.44) |
| ABCC5 Expression | 1.53E−03 | 1.51 (1.17, 1.95) |
| PGR Expression | 0.07 | 0.92 (0.84, 1.01) |
| Hormone Therapy | 0.77 | 1.1 (0.6, 2.02) |

Each of Immune Panels 1, 2, or 3 (or any subset thereof) can be combined with any CCG panel (or any subset thereof) described in this document to yield an embodiment of the disclosure. As an example, according to the CCP data garnered from this Example 4, a new combined immune/CCP panel was constructed from Immune Panel 3 and CCG Panel O to yield the Combined Panel 1 (where “Immune Genes” merely refers to whether the gene is in Table 1) shown in Table 39 below.

TABLE 39
(Combined Panel 1)

| CCP Genes | Immune Genes |
|---|---|
| ASF1B | CCL19 |
| ASPM | CCL5 |
| BUB1B | EVI2B |
| CDCA3 | HCLS1 |
| CDK1 | IGJ |
| CDKN3 | IRF1 |
| CENPF | PTPRC |
| CENPM | |
| DTL | |
| KIAA0101 | |
| KIF11 | |
| KIF20A | |
| MCM10 | |
| NUSAP1 | |
| PBK | |
| PLK1 | |
| PRC1 | |
| PTTG1 | |
| RAD51 | |
| RAD54L | |
| RRM2 | |
| SKA1 | |
| TK1 | |

### Example 5

### Training

The combined CCP/immune gene signature in Table 39, together with additional genes, was trained on a large patient sample cohort to derive a combined model incorporating these molecular components and clinical features to best predict likelihood of distant metastasis-free survival (DMFS) within 10 years of surgery. 459 ER positive, HER2 negative patient samples with complete molecular and clinical data were used in this training analysis. These patients/samples had the following additional characteristics:
Node status: 364 node-negative (“N0”), 95 with one to three nodes (“N1”);
Grade: 133 low, 236 intermediate, 99 high;
Tumor size: Mean=1.7 cm, standard deviation=0.6;
Events: 54 distant metastasis events within 10 years of surgery

The model to be derived would preferably include molecular components and clinical variables that add to the molecular score to provide the most accurate estimate of risk from all available patient data. Coefficients were determined by a multivariate Cox proportional hazards model with 10-year DMFS as the outcome variable. The following modeling components were chosen for training: CCP score (average expression of the CCP genes listed in Table 40 below), Immune score (average expression of the immune genes listed in Table 40 below), ABCC5 gene expression (expression of the ABCC5 gene as represented by the average expression measured by the two assays listed in Table 40 below), PGR gene expression (expression of the PGR gene as represented by the average expression measured by the two assays listed in Table 40 below), tumor size, and node status. Expression of the CCP, immune, ABCC5 and PGR genes was normalized against the average of the housekeeping genes listed in Table 41 below.

TABLE 40
(Combined Panel 2)

| Gene | Gene | | Gene |
|---|---|---|---|
| # | Symbol | Assay ID | Type |
| 1 | ASF1B | Hs00216780_m1 | CCP |
| 2 | ASPM | Hs00411505_m1 | CCP |
| 3 | BUB1B | Hs01084828_m1 | CCP |
| 4 | CDCA3 | Hs00229905_m1 | CCP |
| 5 | CDK1 | Hs00364293_m1 | CCP |
| 6 | CDKN3 | Hs00193192_m1 | CCP |
| 7 | CENPF | Hs00193201_m1 | CCP |
| 8 | CENPM | Hs00608780_m1 | CCP |
| 9 | DTL | Hs00978565_m1 | CCP |
| 10 | KIAA0101 | Hs00207134_m1 | CCP |
| 11 | KIF11 | Hs00189698_m1 | CCP |
| 12 | KIF20A | Hs00993573_m1 | CCP |
| 13 | MCM10 | Hs00960349_m1 | CCP |
| 14 | NUSAP1 | Hs01006195_m1 | CCP |
| 15 | PBK | Hs00218544_m1 | CCP |
| 16 | PLK1 | Hs00153444_m1 | CCP |
| 17 | PRC1 | Hs00187740_m1 | CCP |
| 18 | PTTG1 | Hs00851754_u1 | CCP |
| 19 | RAD51 | Hs00153418_m1 | CCP |
| 20 | RAD54L | Hs00269177_m1 | CCP |
| 21 | RRM2 | Hs00357247_g1 | CCP |
| 22 | SKA1 | Hs00536843_m1 | CCP |
| 23 | TK1 | Hs01062125_m1 | CCP |
| 24 | CCL19 | Hs00171149_m1 | Immune |
| 25 | CCL5 | Hs00174575_m1 | Immune |
| 26 | EVI2B | Hs00272421_s1 | Immune |
| 27 | HCLS1 | Hs00945386_m1 | Immune |
| 28 | IGJ | Hs00950678_g1 | Immune |
| 29 | IRF1 | Hs00971965_m1 | Immune |
| 30 | PTPRC | Hs00894732_m1 | Immune |
| 31 | ABCC5 | Hs00981085_m1; | ABCC5 |
| | | Hs00981087_m1 | |
| 32 | ESR1 | Hs00174860_m1; | ER |
| | | Hs01046815_m1 | |
| 33 | PGR | Hs01556702_m1; | PR |
| | | Hs01556707_m1 | |
| 34 | ERBB2 | Hs01001580_m1; | HER2 |
| | | Hs01001582_m1 | |

TABLE 41

| Gene | | |
|---|---|---|
| Symbol | Assay ID | Gene Type |
| CLTC | Hs00191535_m1 | Housekeeping |
| PPP2CA | Hs00427259_m1 | Housekeeping |
| PSMA1 | Hs00267631_m1 | Housekeeping |
| PSMC1 | Hs02386942_g1 | Housekeeping |
| RPL13A; | Hs03043885_g1 | Housekeeping |
| RPL13AP5 | | |
| RPL8 | Hs00361285_g1 | Housekeeping |
| RPS29 | Hs03004310_g1 | Housekeeping |
| SLC25A3 | Hs00358082_m1 | Housekeeping |
| TXNL1 | Hs00355488_m1 | Housekeeping |

The following Combined Score was derived from this analysis incorporating these components and optimizing their weighting:

Combined Score=(0.54×CCP score)−(0.44×Immune score)+(0.40×ABCC5)−(0.09×PGR)+(0.48×tumor size in cm)+(0.73×node status [0 or 1])

This Combined Score was highly statistically significant, indeed the only independently significant variable, in predicting 10-year DMFS in both univariate and multivariate analysis in this training cohort, as shown in Table 42 below.

TABLE 42

| | HR (95% CI) | p-value |
|---|---|---|
| Univariate Analysis | | |
| Combined score | 2.72 (2.05, 3.65) | 2.0 × 10⁻¹² |
| Multivariate Analysis | | |
| Combined score* | 2.70 (1.89, 3.90) | 3.1 × 10⁻⁸ |
| Age at surgery | 1.01 (0.98, 1.03) | 0.69 |
| Tumor size (cm) | 0.99 (0.62, 1.54) | 0.96 |
| Lymph node status | 1.00 (0.54, 1.81) | 0.99 |

*equivalent to test of the molecular component alone

### Validation

The Combined Score model above was validated on a large patient sample cohort of 559 ER positive, HER2 negative, endocrine therapy treated, chemotherapy naïve breast cancer patients. These patients/samples had the following additional characteristics:
Node status: 299 N0, 259 N1;
Grade: 33 low (“1”), 282 intermediate (“2”), 234 high (“3”);
Tumor size: Mean=2.1 cm, standard deviation=0.92;
Events: 117 (21%) distant metastasis events within 10 years of surgery

The Combined Score was by far the most highly statistically significant variable in predicting 10-year DMFS in both univariate and multivariate analysis in this validation cohort, as shown in Table 43 below.

TABLE 43

| | | HR (95% CI) | p-value |
|---|---|---|---|
| Univariate Analysis | | | |
| Combined score | | 1.64 (1.37, 1.96) | 9 × 10⁻⁸ |
| Multivariate Analysis | | | |
| Combined score* | | 1.82 (1.46, 2.27) | 1.5 × 10⁻⁷ |
| Age at surgery | | 0.98 (0.96, 1.00) | 0.056 |
| Tumor size (cm) | | 0.88 (0.72, 1.07) | 0.21 |
| Lymph node status | | 0.89 (0.61, 1.31) | 0.56 |
| Grade | 1 | 0.18 (0.01, 0.85) | 0.0015 |
| | 2 | — | |
| | 3 | 1.67 (1.11, 2.54) | |

*equivalent to test of the molecular component alone

### Example 6

The CCP, Immune, and Molecular scores, measured by qPCR in Example 4, were measured in this example using a combination of three microarray datasets (Gene Expression Omnibus datasets GSE16716, GSE20271, and GSE32646) to test the CCP Score and Molecular Score's ability to predict chemotherapy effectiveness. The base2 logarithms of the preprocessed intensities were averaged across multiple probes corresponding to the same gene. The summarized gene expressions were subsequently averaged within the CCP and immune gene groups in Table 39 to yield, respectively, a CCP score and Immune score. The Molecular score was calculated by incorporating pre-specified components and weights:

Molecular Score=(0.436×CCP score)−(0.189×Immune score)+(0.155×ABCC5)−(0.086×PGR).

246 unique ER positive, HER2 negative patient samples with complete clinical data were used in this analysis. These patients/samples had the following additional characteristics:
Node status: 81 node-negative, 165 node-positive; 1 unknown (excluded from analysis)
Grade: 32 low, 146 intermediate, 59 high; 10 unknown (excluded from analysis)
Tumor size: 3 T0, 17 T1, 149 T2, 38 T3, and 40 T4;
Events: 12 pathological complete response.

Association of the Molecular Score and the CCP component of the Molecular Score with complete pathological response (pCR) was evaluated by logistic regression. Each score was included in a model with the clinical variables. Both the Molecular Score and the CCP component of the Molecular score were statistically significant, with p-values of 0.029 and 0.015 respectively.

### Example 7

The prognostic value of the CCP gene signature, Molecular signature from Example 6, and Combined Signature from Example 5 was tested on a large patient sample cohort to determine each score's ability to predict chemotherapy effectiveness regardless of ER status. 431 adjuvant chemotherapy and 599 untreated invasive breast cancer patient samples with complete molecular and clinical data were used in this analysis. These patients/samples had the following additional characteristics:
Node status: 619 node-negative, 254 with 1-3 nodes, 126 with 4-9 nodes, 31 with 10 or more nodes;
Grade: 165 low, 299 intermediate, 566 high;
Tumor size: median=1.9 cm, interquartile range=1.0 cm;
Events: 265 distant metastases within 10 years of surgery.

The interactions between adjuvant therapy and each score were tested in individual Cox proportional hazards models with 10-year DMFS as the outcome variable. The tests for these interactions with CCP Score, Molecular Score and Combined Score were highly significant (p-values=0.000016, 0.00002 and 0.00012 respectively). In all cases higher scores predicted higher relative benefit to chemotherapy.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing disclosure has been described in some detail by way of illustration and example for purposes of clarity of understanding, it will be obvious that certain changes and modifications may be practiced within the scope of the appended claims.

1. An in vitro method for determining likelihood of breast cancer recurrence, comprising:
(1) measuring, in a sample obtained from a patient, the expression levels of a panel of genes comprising at least 3 test genes, wherein at least two of said test genes are selected from gene numbers 1 to 23 in Table 40 and at least one of said test genes is selected from gene numbers 24 to 30 in Table 40;
(2) providing a test expression score by (1) weighting the determined expression of each gene in said panel of genes with a predefined coefficient, and (2) combining the weighted expression to provide said test expression score, wherein said test genes are weighted to contribute at least 25% to said test expression score; and either
(3)(a) diagnosing a patient in whose sample said test expression score exceeds a first reference expression score as having an increased likelihood of disease recurrence or having an increased likelihood of chemotherapy response compared to a reference population; or
(3)(b) diagnosing a patient in whose sample said test expression score does not exceed a second reference expression score as not having an increased likelihood of disease recurrence or not having an increased likelihood of chemotherapy response compared to a reference population.

2. The method of claim 1, wherein said test genes are weighted to contribute at least 30% of the total weight given to the expression of all of said panel of genes in said test expression score.

3. The method of claim 1, wherein said test genes comprise at least gene numbers 1 through 30 of Table 40.

4. The method of claim 1, wherein said test genes comprise at least gene numbers 1 through 31 of Table 40.

5. The method of claim 1, wherein said test genes comprise the genes listed in Table 40.

6. The method of claim 3, wherein said test genes further comprise at least one of gene numbers 31 through 34 in Table 40.

7. The method of claim 7, wherein said test genes further comprise ABCC5.

8. The method of claim 1, wherein said first and second reference expression scores are the same.

9. The method of claim 9, wherein half of breast cancer patients in said reference population have an expression score exceeding said first reference expression score and half of breast cancer patients in said reference population have an expression score not exceeding said first reference expression score.

10. The method of claim 1, wherein one third of breast cancer patients in said reference population have an expression score exceeding said first reference expression score and one third of breast cancer patients in said reference population have an expression score not exceeding said second reference expression score.

11. The method of claim 10, comprising (a) diagnosing a patient in whose sample said test expression score exceeds said first reference expression score as having an increased likelihood of disease recurrence or having an increased likelihood of chemotherapy response compared to said reference population; (b) diagnosing a patient in whose sample said test expression score does not exceed said second reference expression score as having an increased likelihood of disease recurrence or having an increased likelihood of chemotherapy response compared to said reference population; or (c) diagnosing a patient in whose sample said test expression score exceeds said second reference expression score but does not exceed said first reference expression score as having no increased likelihood of disease recurrence or having no increased likelihood of chemotherapy response compared to said reference population.

12. The method of claim 1, wherein disease recurrence is chosen from the group consisting of distant metastasis of the primary breast cancer; local metastasis of the primary breast cancer; recurrence of the primary breast cancer; progression of the primary breast cancer; and development of locally advanced, metastatic disease.

13. The method of claim 1, wherein chemotherapy response is pathological complete response.

14. A method for determining a breast cancer patient's likelihood of breast cancer recurrence, comprising:
(1) measuring, in a sample obtained from said patient, the expression levels of a panel of genes comprising at least 3 test genes selected from Table 40, wherein at least two of said test genes are CCP genes listed in Table 40 and at least one of said test genes is an immune gene listed in Table 40;
(2) providing a test expression score by (1) weighting the determined expression of each gene in said panel of genes with a predefined coefficient, and (2) combining the weighted expression to provide said test expression score, wherein said test genes are weighted to contribute at least 25% to said test expression score;
(3) providing a test prognostic score combining said test expression score with at least one test clinical score representing at least one clinical variable; and
(4) diagnosing said patient as having either (a) an increased likelihood of breast cancer recurrence based at least in part on said test prognostic score exceeding a first reference prognostic score or (b) no increased likelihood of breast cancer recurrence based at least in part on said test prognostic score not exceeding a second reference prognostic.

15. The method of claim 14, wherein said at least one clinical score incorporates at least one clinical variable chosen from the group consisting of node status, tumor size and tumor grade.

16. The method of claim 15, wherein said prognostic scores incorporate (a) a first clinical score representing node status and (b) a second clinical score representing tumor size.

17. The method of claim 16, wherein a patient's node status is negative (N0) if said patient was found to have no positive lymph nodes and positive (N1) if said patient was found to have between one and three positive lymph nodes.

18. The method of claim 16, wherein the value for said second clinical score is the size of the tumor in centimeters.

19. The method of claim 14, said prognostic scores are calculated according to a formula comprising the following terms: (D×Tumor Size)+(E×node status)+(B×CCP score)−(A×Immune score)+(C×ABCC5).

20. The method of claim 14, said prognostic scores are calculated according to a formula comprising the following terms: (D×Tumor Size [cm[)+(E×node status [0 or 1])+(B×CCP score)−(A×Immune score)+(C×ABCC5)−(F×PGR).

21. The method of claim 20, said prognostic scores are calculated according to a formula comprising the following terms: (0.54×CCP score)−(0.44×Immune score)+(0.40×ABCC5)−(0.09×PGR)+(0.48×Tumor Size [cm])+(0.73×node status [0 or 1]).

22. A method of determining the prognosis of a patient having breast cancer or the likelihood of cancer recurrence in said patient, comprising:
(1) determining, in a sample obtained from said patient, the expression levels of a panel of genes comprising at least 2, 3, 4, 5, 10, 15, or 20 test genes selected from any of Tables 1 to 10 or Tables 39 or 40;
(2) providing a test value by (1) weighting the determined expression of each gene in said panel of genes with a predefined coefficient, and (2) combining the weighted expression to provide said test value, wherein said test genes are weighted to contribute at least 25%, 50%, 75%, 85% or at least 95% to said test value; and
(3) determining the prognosis using said test value.

23. The method of claim 22, wherein the combined weight given to said test genes is at least 40% of the total weight given to the expression of all of said panel of genes.

24. The method of claim 22, wherein said determining step comprises:
measuring the amount of mRNA in said tumor sample transcribed from each of between 6 and 200 genes; and
measuring the amount of mRNA of one or more housekeeping genes in said tumor sample.

25. The method of claim 22, further comprising comparing said test value to a reference value, wherein a correlation to a poor prognosis is made if said test value is greater than said reference value.
